(19) United States
(12) Patent Application Publication
(10) Pub. No.: US 2025/0057959 A1
(43) Pub. Date: Feb. 20, 2025

(54) PHOSPHANE GOLD(I)-N-HETEROCYCLIC CARBENE COMPLEXES FOR TREATING CANCER

(71) Applicant: KING FAHD UNIVERSITY OF PETROLEUM AND MINERALS, Dhahran (SA)

(72) Inventors: Adam Ahmed Abdallah SULAIMAN, Dhahran (SA); Anvarhusien A. ISAB, Dhahran (SA)

(73) Assignee: KING FAHD UNIVERSITY OF PETROLEUM AND MINERALS, Dhahran (SA)

(21) Appl. No.: 18/450,014

(22) Filed: Aug. 15, 2023

(51) Int. Cl. A61K 47/54; A61P 35/00

(52) U.S. Cl. CPC A61K 47/548; A61K 47/545; A61P 35/00

(57) ABSTRACT

A complex for treating a cancer includes gold, a 1,3-bis(2,6-di-isopropylphenyl)imidazole-2-ylidene ligand, and at least one phosphane ligand selected from the group consisting of bis(2-cyanoethyl)phenylphosphane, (1R,2R)-2-diphenylphosphano)-1 aminocyclohexane, (1R,2R)-2-diphenylphosphano)-1,2-diphenylethylamine, (R)-2 (diphenylphosphano)-1-phenylethylamine, (R)-1-(diphenylphosphano)-2-amino-3,3 dimethylbutane, tricyclohexylphosphane, and 3-(diphenylphosphano)propylamine), wherein the 1,3-bis(2,6-di-isopropylphenyl)imidazole-2-ylidene ligand and the at least one phosphane ligand are bonded to the gold.

### STATEMENT REGARDING PRIOR DISCLOSURE BY THE INVENTORS

Aspects of the present disclosure were described in an article titled “Design, Synthesis, and Preclinical Activity in Ovarian Cancer Models of New Phosphanegold(I)-N-heterocyclic Carbene Complexes” published in Issue 21, Journal of Medicinal Chemistry, which is incorporated herein by reference in its entirety.

### STATEMENT OF ACKNOWLEDGEMENT

Support provided by the Interdisciplinary Research Center for Advanced Materials, King Fahd University of Petroleum and Minerals, Saudi Arabia, through Project INAM2210 is gratefully acknowledged.

Support from the Italian Association for Cancer Research and Italian Ministry of Health under IG 15844 is gratefully acknowledged.

### BACKGROUND

### Technical Field

The present disclosure is directed to gold(I) complexes of N-heterocyclic carbenes, more particularly, phosphane gold(I)-N-heterocyclic carbene complexes, pharmaceutical composition containing the gold(I) complexes, methods of treating cancer by administering the gold(I) complexes and methods of making the gold(I) complexes.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

The investigation of anticancer activity of gold(I) complexes of N-heterocyclic carbenes (NHCs) is a rapidly growing field of research in organometallic chemistry. This may be correlated to their enhanced stability under biological conditions that arise on the basis of strong σ-donation of NHCs, which is comparable to that of phosphanes. The lipophilicity of the gold-NHCs could be tuned more easily by modulating the nature of the substituents of the heterocyclic rings. The antirheumatic gold(I)-phosphane drug auranofin [(2,3,4,6-tetra-O-acetyl-L-thio-β-D-glycopyranosato-S-)-(triethylphosphane)gold(I)] is often compared as a standard for anticancer gold(I) compounds. In recent years, several gold(I)-phosphane complexes have been shown to exhibit anti-tumor activity. The crystal structure analyses of NHC-gold(I) complexes reveal a linear geometry around gold(I). Some of these complexes are characterized by aurophilic interactions.

NHC analogs of auranofin containing carbenes and tetracetylthioglucose as ligands and a corresponding crystal structure have been disclosed [Baker, M. V.; Barnard, P. J.; Berners-Price, S. J.; Brayshaw, S. K.; Hickey, J. L.; Skelton, B. W.; White, A. H. Synthesis and Structural Characterisation of Linear Au(I)N-Heterocyclic Carbene Complexes: New Analogues of the Au(I) Phosphine Drug Auranofin. J. Organomet. Chem. 2005, 690 (24), 5625-5635]. Two such complexes [Liu, W.; Bensdorf, K.; Proetto, M.; Hagenbach, A.; Abram, U.; Gust, R. Synthesis, Characterization, and in Vitro Studies of Bis[1,3-Diethyl-4,5-Diarylimidazol-2-Ylidene]Gold(IIII) Complexes. J. Med. Chem. 2012, 55 (8), 3713-3724] were found to be more effective as compared to cisplatin or 5-fluorouracil. Reports on the anticancer potential of the mixed ligand gold(I) complexes of carbenes and phosphanes show complexes which display antiproliferative properties against different human cancer cell lines; particularly, [Rubbiani, R.; Can, S.; Kitanovic, I.; Alborzinia, H.; Stefanopoulou, M.; Kokoschka, M.; Monchgesang, S.; Sheldrick, W. S.; Wolfi, S.; Ott, I. Comparative in Vitro Evaluation of N-Heterocyclic Carbene Gold(I) Complexes of the Benzimidazolylidene Type. J. Med. Chem. 2011, 54 (24), 8646-8657; Rubbiani, R.; Salassa, L.; de Almeida, A.; Casini, A.; Ott, I. Cytotoxic Gold(I)N-Heterocyclic Carbene Complexes with Phosphane Ligands as Potent Enzyme Inhibitors. ChemMedChem 2014, 9(6), 1205-1210; and Holenya, P.; Can, S.; Rubbiani, R.; Alborzinia, H.; Junger, A.; Cheng, X.; Ott, I.; Wolfi, S. Detailed Analysis of Pro-Apoptotic Signaling and Metabolic Adaptation Triggered by a N-Heterocyclic Carbene-Gold(I) Complex. Metallomics 2014, 6 (9), 1591-1601] a comparative cytotoxic evaluation of a series of benzimidazole-derived N-heterocyclic carbene gold(I) complexes with phosphane as co-ligands.

Mechanistic studies on gold-carbene compounds demonstrated that their anticancer activity is due to the induction of mitochondrial membrane permeabilization and the inhibition of protein activities. The inhibition of the mitochondrial enzyme thioredoxin reductase (TrxR) plays a role in the anticancer activity of gold complexes. The antimitochondrial activity perturbs cellular regulation systems, such as the antioxidant network, and induces apoptosis via the intrinsic apoptotic pathway. A series of gold(I) complexes with benzimidazole-derived NHC ligands [Rubbiani, R.; Kitanovic, I.; Alborzinia, H.; Can, S.; Kitanovic, A.; Onambele, L. A.; Stefanopoulou, M.; Geldmacher, Y.; Sheldrick, W. S.; Wolber, G.; Prokop, A.; Wolfi, S.; Ott, I. Benzimidazol-2-Ylidene Gold(I) Complexes Are Thioredoxin Reductase Inhibitors with Multiple Antitumor Properties. J. Med. Chem. 2010, 53 (24), 8608-8618, incorporated herein by reference in its entirety] displayed potent antiproliferative activity and selectively targeted TrxR.

In developed countries, ovarian cancer (OvCa) represents the most common cause of death among gynecologic malignancies. The main treatments are debulking surgery followed by platinum-taxane chemotherapy, but even though patients are responsive at first, they eventually relapse due to the development of acquired platinum resistance. Advanced epithelial ovarian cancer is characterized by the buildup of fluid in the abdomen, the malignancy-related ascites, that contains aggregates of cancer cells or multicellular tumor spheroids (MCTSs). MCTSs derived from the primary tumor can disseminate and form metastasis which may be resistant to anticancer drugs. MCTSs, characterized by an intermediate complexity between two-dimensional (2D) monolayers and in vivo solid tumors, represent a three-dimensional (3D) model for preclinical cancer research. Finding new molecules active against cisplatin-resistant OvCa cells and active in 3D-MCTSs is a challenge, with these new drugs being an alternative treatment strategy for relapsed, recurrent diseases.

The antitumor effects of gold(I) complexes comprising the carbene ligand 1,3-bis(2,6-diisopropylphenyl)imidazol-2-ylidene (IPr) in the presence of different types of co-ligands, which include thiones, dithiocarbamates, and selenones, have been explored in the past.

However, there still exists a need to develop novel complexes with enhanced lipophilicity and increased cellular uptake. Accordingly, an object of the present disclosure is directed toward the preparation and evaluation of cytotoxic gold(I) complexes based on IPr and phosphane ligands with enhanced lipophilicity and cellular uptake.

### SUMMARY

In an exemplary embodiment, a complex is described. The complex includes gold; a 1,3-bis(2,6-di-isopropylphenyl)imidazole-2-ylidene ligand; and at least one phosphane ligand selected from the group consisting of bis(2-cyanoethyl)phenylphosphane, (1R,2R)-2-diphenylphosphano)-1-aminocyclohexane, (1R,2R)-2-diphenylphosphano)-1,2-diphenylethylamine, (R)-2-(diphenylphosphano)-1-phenylethylamine, (R)-1-(diphenylphosphano)-2-amino-3,3-dimethylbutane, tricyclohexylphosphane, and 3-(diphenylphosphano)propylamine), wherein the 1,3-bis(2,6-di-isopropylphenyl)imidazole-2-ylidene ligand and the at least one phosphane ligand are bonded to the gold.

In some embodiments, the phosphane ligand is tricyclohexylphosphane.

In some embodiments, the complex has a half maximal inhibitory concentration of 0.1 to 2.5 micromolar (μM) in a first ovarian cancer cell line, A2780.

In some embodiments, the complex has a half maximal inhibitory concentration of 0.1 to 4.0 μM in a first ovarian cancer cell line resistant to cisplatin, A2780cis.

In some embodiments, the complex has a fold resistance of 0.5 to 2, wherein the fold resistance is based on a ratio of a half maximal inhibitory concentration in the first ovarian cancer cell line resistant to cisplatin, A2780cis, to a half maximal inhibitory concentration in the first ovarian cancer cell line, A2780.

In some embodiments, when the phosphane ligand is tricyclohexylphosphene, the complex has a half maximal inhibitory concentration of 0.10 to 0.20 μM in the first ovarian cancer cell line, A2780.

In some embodiments, when the phosphane ligand is tricyclohexylphosphene, the complex has a half maximal inhibitory concentration of 0.10 to 0.15 μM in the first ovarian cancer cell line resistant to cisplatin, A2780cis.

In some embodiments, when the phosphane ligand is tricyclohexylphosphene, the complex has a fold resistance of less than 1. The fold resistance is based on a ratio of a half maximal inhibitory concentration in the first ovarian cancer cell line resistant to cisplatin, A2780cis, to a half maximal inhibitory concentration in the first ovarian cancer cell line, A2780.

In some embodiments, the complex has a half maximal inhibitory concentration of 0.55 to 0.65 μM in a second ovarian cancer cell line, SKOV3.

In an exemplary embodiment, a pharmaceutical composition is described. The pharmaceutical composition, including the complex, is present in an amount effective for treating a patient having ovarian cancer, along with at least one pharmaceutical additive or adjuvant.

In an exemplary embodiment, a process for making the complex is described. The process includes mixing a silver hexafluorophosphate salt in a polar protic solvent with a 1,3-bis(2,6-diisopropylphenyl-imidazole-2-ylidene) gold chloride salt in a polar aprotic solvent to form a reaction mixture; and reacting the reaction mixture with the phosphane ligand in a polar aprotic solvent to form the complex. The silver hexafluorophosphate salt, the 1,3-bis(2,6-diisopropylphenyl-imidazole-2-ylidene) gold chloride salt, and the phosphane ligands are in an equimolar amount.

In an exemplary embodiment, a method for treating ovarian cancer is described. The method includes administering the complex to a patient in need of treatment for ovarian cancer. The phosphene ligand in the complex is tricyclohexylphosphane. During administering, the complex is contacted with an ovarian cancer in the form of at least one of a monolayer of cells, a multicellular tumor spheroid (MCTS), and an in vivo xenograft tumor.

In some embodiments, administering the complex having induces apoptosis in A2780, A2780cis, and a multicellular tumor spheroid sample of the second ovarian cancer cell line, SKOV3, wherein the phosphene ligand in the complex is tricyclohexylphosphane.

In some embodiments, the complex administered at a concentration of 1 μM decreases the volume of SKOV3-MCTSs by 85 to 95% based on volume after 7 days.

In some embodiments, the complex administered at a concentration of 0.50 μM generates mitochondrial reactive oxygen species (mitROS) in SKOV3-MCTSs of 50 to 60% based on a cell count after 24 hours, wherein the phosphene ligand in the complex is tricyclohexylphosphane.

In some embodiments, the complex administered to the sample at a concentration of 0.1 to 0.6 μM has an amount of cells in the S phase from 15 to 35% compared to the sample in the absence of the complex having an amount of cells in the S phase from 40 to 50% based on a cell count after a time of 24 hours, wherein the phosphene ligand in the complex is tricyclohexylphosphane.

In some embodiments, the complex administered to the sample at a concentration of 0.1 to 0.6 μM has an amount of cells in the G1 phase from 60 to 80% compared to the sample in the absence of the complex having an amount of cells in the G1 phase from 45 to 55% based on a cell count after a time of 24 hours, wherein the phosphene ligand in the complex is tricyclohexylphosphane.

In some embodiments, the complex administered to the sample at a concentration of 0.5 μM decreases proteasome activity by 30 to 70% after 24 hours based on a 20S-proteasome assay compared to the sample in the absence of the complex, wherein the phosphene ligand in the complex is tricyclohexylphosphane.

In some embodiments, the sample is in vivo xenograft tumor, and the complex administered to the sample showed a reduction of the sample of more than 80% based on sample volume after 35 days of treatment of 2 milligrams of the complex per kilogram of a body weight of a host of the sample two times a week. The phosphene ligand in the complex is tricyclohexylphosphane.

In some embodiments, a pharmaceutical composition including the complex or a pharmaceutically acceptable salt thereof is in combination with a pharmaceutically acceptable carrier, diluent, or excipient.

These and other aspects of the non-limiting embodiments of the present disclosure will become apparent to those skilled in the art upon review of the following description of specific non-limiting embodiments of the disclosure in conjunction with the accompanying drawings. The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure (including alternatives and/or variations thereof) and many of the attendant advantages thereof will be readily obtained as the same becomes better understood by reference to the following detailed description when considered in connection with the accompanying drawings, wherein:

FIG. 1A is a flowchart depicting a method of preparing a complex of the present disclosure, according to certain embodiments;

FIG. 1B is a schematic illustration depicting the activity of the complex of the present disclosure in ovarian cancer, according to certain embodiments;

FIG. 2A-2G is a schematic illustration depicting chemical structures of complexes 1-7, respectively, according to certain embodiments;

FIGS. 3A-3G depict high-resolution mass spectrometry analysis of gold (I) complexes 1-7, respectively, according to certain embodiments;

FIG. 4 depicts proton (¹H) Nuclear Magnetic Resonance (NMR) spectra of complex 1, in CDCl₃/DMSO-d₆, according to certain embodiments;

FIG. 5 depicts carbon (¹³C) NMR spectra of complex 1, in CDCl₃/DMSO-d₆, according to certain embodiments;

FIG. 6 depicts phosphorous (³¹P) NMR spectra of complex 1, in CDCl₃/DMSO-d₆, according to certain embodiments;

FIG. 7 depicts ¹H NMR spectra of complex 2, in CDCl₃, according to certain embodiments;

FIG. 8 depicts ¹³C NMR spectra of complex 2, in CDCl₃, according to certain embodiments;

FIG. 9 depicts ³¹P NMR spectra of complex 2, in CDCl₃, according to certain embodiments;

FIG. 10 depicts ¹H NMR spectra of complex 3, in CDCl₃, according to certain embodiments;

FIG. 11 depicts ¹³C NMR spectra of complex 3, in CDCl₃, according to certain embodiments;

FIG. 12 depicts ³¹P NMR spectra of complex 3, in CDCl₃, according to certain embodiments;

FIG. 13 depicts ¹H NMR spectra of complex 4, in CDCl₃, according to certain embodiments;

FIG. 14 depicts ¹³C NMR spectra of complex 4, in CDCl₃, according to certain embodiments;

FIG. 15 depicts ³¹P NMR spectra of complex 4, in CDCl₃, according to certain embodiments;

FIG. 16 depicts ¹H NMR spectra of complex 5, in CDCl₃, according to certain embodiments;

FIG. 17 depicts ¹³C NMR spectra of complex 5, in CDCl₃, according to certain embodiments;

FIG. 18 depicts ³¹P NMR spectra of complex 5, in CDCl₃, according to certain embodiments;

FIG. 19 depicts ¹H NMR spectra of complex 6, in CDCl₃, according to certain embodiments;

FIG. 20 depicts ¹³C NMR spectra of complex 6, in CDCl₃, according to certain embodiments;

FIG. 21 depicts ³¹P NMR spectra of complex 6, in CDCl₃, according to certain embodiments;

FIG. 22 depicts ¹H NMR spectra of complex 7, in CDCl₃, according to certain embodiments;

FIG. 23 depicts ¹³C NMR spectra of complex 7, in CDCl₃, according to certain embodiments;

FIG. 24 depicts ³¹P NMR spectra of complex 7, in CDCl₃, according to certain embodiments;

FIG. 25 shows a view of a molecular structure of a solvated molecule of complex 6, with atom labelling, according to certain embodiments.

FIG. 26 shows a crystal packaging of complex 6, according to certain embodiments;

FIG. 27A shows dose-response curves performed after 72 hours treatment with increasing concentrations of complex 6 in A2780, A2780cis, and SKOV3 cells, according to certain embodiments;

FIG. 27B shows dose-response curves performed after 72 hours treatment with increasing concentrations of cisplatin in A2780, A2780cis, and SKOV3 cells, according to certain embodiments;

FIG. 27C shows dose-response curves performed after 72 hours treatment with increasing concentrations of auranofin in A2780, A2780cis, and SKOV3 cells, according to certain embodiments;

FIG. 27D shows dose-response curves performed after 72 hours treatment with increasing concentrations of complex 6 in adipose-derived stem cells (ADSCs), according to certain embodiments;

FIG. 28A shows cell viability assays performed after 72 hours treatment with increasing concentrations of complex 6 in ovarian cancer (OvCa) cell lines (A2780, A2780cis, and SKOV3), according to certain embodiments;

FIG. 28B shows representative photomicrographs of OvCa cells treated for 72 hours with increasing concentrations of complex 6 and then stained with crystal violet, according to certain embodiments;

FIG. 29A shows complex 6 induced apoptosis, mitochondrial reactive oxygen species (mitROS), and DNA double-strand breaks (DNA DSBs) in ovarian cancer cells, A2780 and A2780cis, on treatment for 48 hours, double stained with annexin-V-FITC and 7AAD, and analyzed by flow cytometry, according to certain embodiments;

FIG. 29B shows effect of complex 6 on apoptosis (caspase-3,7 activation assay), in A2780 and A2780cis, as evaluated by flow cytometry, with fluorochrome-labeled inhibitors of caspases (FLICA), according to certain embodiments;

FIGS. 29C and 29D show complex 6 induced BAX- and Bcl-2-fluorescence intensity expression, respectively, evaluated by flow cytometry, according to certain embodiments;

FIG. 29E and FIG. 29F shows an effect of complex 6 on mitROS generation, detected with MitoSOX red mitochondrial superoxide indicator, and their corresponding dot plots, respectively, according to certain embodiments;

FIG. 29G shows a western blot for evaluating the effect of complex 6 on DNA DSBs as demonstrated by the phosphorylation of histone H2A.X (γ-H2AX) and α-tubulin protein expression in A2780 and A2780cis, according to certain embodiments;

FIG. 30A shows flow cytofluorimetric dot plots showing the effect of complex 6 on apoptosis (annexin-V-fluorescein isothiocyanate (annexin-V-FITC) and 7AAD staining) in A2780 and A2780cis cells, after treatment in a dose-dependent manner, according to certain embodiments;

FIG. 30B shows flow cytofluorimetric dot plots showing the effect of complex 6 on apoptosis (annexin-V-FITC and 7AAD staining) in SKOV3-MCTSs, after treatment in a dose-dependent manner, according to certain embodiments;

FIG. 31A shows effect of complex 6 on spheroid volume in 3D-SKOV3-MCTSs, according to certain embodiments;

FIG. 31B shows representative phase-contrast microphotographs showing the spheroid volume decrease by complex 6 (original magnification 4×), according to certain embodiments;

FIG. 31C shows cell viability of SKOV3-MCTSs after treatment with complex 6 for 72 hours, according to certain embodiments;

FIG. 31D shows effect of complex 6 on inducing apoptosis (annexin-V-FITC and 7AAD staining, revealed by flow cytometry) in SKOV3-MCTSs, according to certain embodiments;

FIG. 31E shows representative phase-contrast microphotographs showing the cytotoxic effects of complex 6 in multiple sizes of SKOV3-MCTSs, according to certain embodiments;

FIG. 31F shows effect of complex 6 on caspase-3,7 activation, evaluated with FLICA, in 3D-SKOV3-MCTSs, according to certain embodiments;

FIG. 31G and FIG. 31H shows effect of complex 6 after treatment for 24 hours on mitROS generation, in 3D-SKOV3-MCTSs, and their corresponding dot plats, respectively, according to certain embodiments;

FIG. 31I shows a western blot for evaluating the effect of complex 6 on phospho-histone H2A.X (γ-H2AX) and α-tubulin protein expression, in 3D-SKOV3-MCTSs, according to certain embodiments;

FIG. 32A is a bar chart showing the percentage of cells in each cell cycle phase in A2780, A2780cis, and SKOV3 cells, according to certain embodiments;

FIG. 32B shows representative cytofluorimetric histograms of the cell cycle progression after treatment with complex 6, according to certain embodiments;

FIG. 32C shows a western blot of molecules (CDk2, CDk4, CDk6, CyCE, CycD1, CycD3, CycE, E2F1, pRB, and Vinculin) involved in cell cycle regulation in A2780, A2780cis, and SKOV3 cells, according to certain embodiments;

FIG. 32D shows effect of concentration of complex 6 on 20S-proteasome activity in A2780, A2780cis, and SKOV3 cells, according to certain embodiments;

FIG. 32E shows effect of complex 6 in thioredoxin reductase (TrxR) enzymatic activity in A2780, A2780cis, and SKOV3 cells, according to certain embodiments;

FIG. 33 is a western blot depicting an expression of molecules that regulate cell cycle progression from G0/G1 to S phase after treating OvCa cells with complex 6 for 24 hours, according to certain embodiments;

FIG. 34A shows effect of cisplatin on apoptosis (annexin-V-FITC and 7AAD staining) in OvCa cells, A2780 and A2780cis, after treatment for 48 hours, according to certain embodiments;

FIG. 34B shows effect of cisplatin on apoptosis by caspase-3,7 activation, evaluated with FLICA, according to certain embodiments;

FIG. 34C shows a western blot for evaluating the effect of cisplatin on phospho-histone H2A.X (7-H2AX) and α-tubulin protein expression in A2780 and A2780cis cells, according to certain embodiments;

FIG. 34D shows effect on cisplatin on mitROS generation in A2780 and A2780cis cells, according to certain embodiments;

FIG. 34E shows cytofluorimetric histograms of the cell cycle progression (the percentage of cells in each cell cycle phase were depicted) on treating OvCa cells with cisplatin for 24 hours, according to certain embodiments;

FIG. 34F shows phase-contrast microphotographs showing the spheroid volume decrease by cisplatin (original magnification 4×) in SKOV3-MCTSs, according to certain embodiments;

FIG. 34G is a bar graph showing spheroid volume decrease by cisplatin in SKOV3-MCTSs, according to certain embodiments;

FIG. 35A shows effect of auranofin on apoptosis (annexin-V and 7AAD staining) in OvCa cells, A2780 and A2780cis, after treatment for 48 hours, according to certain embodiments;

FIG. 35B shows effect of auranofin on apoptosis by caspase-3,7 activation, evaluated with FLICA, according to certain embodiments;

FIG. 35C shows cell cycle progression (the percentage of cells in each cell cycle phase were depicted) on treating OvCa cells with auranofin for 24 hours, according to certain embodiments;

FIG. 35D shows effect on auranofin on mitROS generation in A2780 and A2780cis, according to certain embodiments;

FIG. 35E shows a western blot for evaluating the effect of auranofin on phospho-histone H2A.X (γ-H2AX) and α-tubulin protein expression in A2780 and A2780cis cells, treated with auranofin, according to certain embodiments;

FIG. 35F shows phase-contrast microphotographs showing the spheroid volume increase by auranofin (original magnification 4×) in SKOV3-MCTSs, according to certain embodiments;

FIG. 35G is a bar graph showing spheroid volume increase by auranofin in SKOV3-MCTSs, according to certain embodiments;

FIG. 36A shows the effect of auranofin concentrations after 72 hours in SKOV3-MCTSs, according to certain embodiments;

FIG. 36B shows phase-contrast microphotographs showing the spheroid volume decrease by auranofin in SKOV3-MCTSs, according to certain embodiments;

FIG. 37A shows representative photomicrographs of the wound-healing assay performed on SKOV3 cell layers treated for 24 hours with complex 6, according to certain embodiments;

FIG. 37B is a bar chart showing cell-free area remaining over time as cells migrated into wound, normalized to time 0, according to certain embodiments;

FIG. 37C shows immunofluorescent micrographs of SKOV3 cells treated for 24 hours with complex 6 cells and stained with F-actin, tubulin, and nuclei (topro-3), according to certain embodiments;

FIG. 38A shows effect of complex 6 in OvCa xenograft tumor growth in athymic nude mice, according to certain embodiments;

FIG. 38B shows representative images of extracted tumors and bar charts showing tumor weight from treated with complex 6 and untreated xenografted mice, according to certain embodiments; and

FIG. 38C shows body weight of xenografted mice after treatment with complex 6, according to certain embodiments.

### DETAILED DESCRIPTION

In the following description, it is understood that other embodiments may be utilized, and structural and operational changes may be made without departure from the scope of the present embodiments disclosed herein.

Reference will now be made to specific embodiments or features, examples of which are illustrated in the accompanying drawings. In the drawings, whenever possible, corresponding or similar reference numerals will be used to designate identical or corresponding parts throughout the several views. Moreover, references to various elements described herein are made collectively or individually when there may be more than one element of the same type. However, such references are merely exemplary in nature. It may be noted that any reference to elements in the singular may also be constructed to relate to the plural and vice-versa without limiting the scope of the disclosure to the exact number or type of such elements unless set forth explicitly in the appended claims. In the drawings, like reference numerals designate identical or corresponding parts throughout the several views. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

As used herein, the term “cancer” refers to any one of a large number of diseases characterized by the development of abnormal cells that divide uncontrollably and have the ability to infiltrate and destroy normal body tissue, and may refer to all types of cancer, neoplasm, or malignant tumors found in mammals (e.g., humans), including leukemias, lymphomas, carcinomas, and sarcomas, e.g., proliferative disorders. Exemplary cancers that may be treated with the method or composition provided herein include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with the method or composition provided herein include cancer of the thyroid, endocrine system, brain, breast, cervix, colon, head and neck, liver, kidney, lung, ovary, pancreas, rectum, stomach, and uterus. Additional examples include, but are not limited to, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulinoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

As used herein, the term “half maximal inhibitory concentration (IC₅₀)” refers to the measure of the potency of a substance in inhibiting a specific biological or biochemical function. IC₅₀ is a quantitative measure that indicates how much of a particular inhibitory substance (e.g., drug) is needed to inhibit, in vitro, a given biological process or biological component by 50%. The biological component can be an enzyme, cell, cell receptor, microorganism, and the like. IC₅₀ values are typically expressed as molar concentration.

As used herein, “analogue” and “analog” refers to a chemical compound that is structurally similar to a parent compound, but differs slightly in composition (e.g., one atom or functional group is different, added, or removed). The analogue may or may not have different chemical or physical properties than the original compound and may or may not have improved biological and/or chemical activity. For example, the analogue may be more hydrophilic, or it may have altered reactivity as compared to the parent compound. The analogue may mimic the chemical and/or biologically activity of the parent compound (i.e., it may have similar or identical activity), or, in some cases, may have increased or decreased activity. The analogue may be a naturally or non-naturally occurring variant of the original compound. Other types of analogues include isomers (enantiomers, diastereomers, and the like) and other types of chiral variants of a compound, as well as structural isomers.

As used herein, “derivative” refers to a chemically or biologically modified version of a chemical compound that is structurally similar to a parent compound and (actually or theoretically) derivable from that parent compound. A “derivative” differs from an “analogue” in that a parent compound may be the starting material to generate a “derivative,” whereas the parent compound may not be used as the starting material to generate an “analogue.” A derivative may or may not have different chemical or physical properties of the parent compound. For example, the derivative may be more hydrophilic, or it may have altered reactivity as compared to the parent compound. Derivatization (i.e., modification) may involve the substitution of one or more moieties within the molecule (e.g., a change in a functional group). The term “derivative” also includes conjugates, and prodrugs of a parent compound (i.e., chemically modified derivatives that can be converted into the original compound under physiological conditions). A “prodrug” is meant to indicate a compound that can be converted under physiological conditions or by solvolysis to a biologically active compound. Thus, the term “prodrug” refers to a precursor of a biologically active compound that is pharmaceutically acceptable. A prodrug may be inactive when administered to a subject, but is converted in vivo to an active compound, for example, by hydrolysis.

The term “therapeutically effective amount” as used herein refers to the amount of the complex being administered which will relieve to some extent one or more of the symptoms of the disorder being treated. A therapeutically effective amount may be at a level that will exercise the desired effect. In reference to cancer or pathologies related to increased cell division, a therapeutically effective amount refers to that amount which has the effect of at least one of the following: (1) reducing the size of a tumor, (2) inhibiting (that is, slowing to some extent, preferably stopping) aberrant cell division, growth or proliferation, for example, cancer cell division, (3) preventing or reducing the metastasis of cancer cells, (4) relieving to some extent (or, preferably, eliminating) one or more symptoms associated with a pathology related to or caused in part by unregulated or aberrant cellular division, including for example, cancer and (5) inducing apoptosis of cancer cells or tumor cells.

As used herein, the terms “therapies” and “therapy” can refer to any method(s), composition(s), and/or agent(s), and/or complexes that can be used in the prevention, treatment, and/or management of a cancer or one or more symptoms thereof. Therapy may include immunotherapy, radiation therapy, drug therapy, chemotherapy, combination therapy, and the like.

As used herein, the terms “treat,” “treatment,” and “treating” in the context of the administration of a therapy to a subject in need thereof refer to the reduction or inhibition of the progression and or duration of cancer, the reduction or amelioration of the severity of cancer, and/or the amelioration of one or more symptoms thereof resulting from the administration of one or more therapies. In some embodiments, the subject is a mammalian subject. In one embodiment, the subject is a mouse. In another embodiment, the subject is a human. “Treating” or “treatment” of a disease includes preventing the disease from occurring in a subject that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to cancer or hyperplasia, these terms simply mean that the life expectancy of an individual affected with cancer will be increased or that one or more of the symptoms of the disease will be reduced. In specific embodiments, such terms refer to one, two, three, or more results following the administration of one, two, three, or more therapies: (1) a stabilization, reduction, or elimination of the cancer stem cell population; (2) a stabilization, reduction, or elimination in the cancer cell population; (3) a stabilization or reduction in the growth of a tumor or neoplasm; (4) an impairment in the formation of a tumor; (5) eradication, removal, or control of primary, regional, and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate; (10) a decrease in hospitalization lengths; (11) the size of the tumor is maintained and does not increase or increases by less than 10%, preferably less than 5%, preferably less than 4%, preferably less than 2%; and, (12) an increase in the number of patients in remission. In certain embodiments, such terms refer to a stabilization or reduction in cancer stem cell population. In some embodiments, such terms refer to a stabilization or reduction in the growth of cancer cells. In some embodiments, such terms refer to stabilization or reduction in cancer stem cell population and a reduction in the cancer cell population. In some embodiments, such terms refer to a stabilization or reduction in the growth and or formation of a tumor. In some embodiments, such terms refer to the eradication, removal, or control of primary, regional, or metastatic cancer (e.g., the minimization or delay of the spread of cancer). In some embodiments, such terms refer to a reduction in mortality and/or an increase in the survival rate of a patient population. In further embodiments, such terms refer to an increase in the response rate, the durability of response, or the number of patients who respond or are in remission. In some embodiments, such terms refer to a decrease in the hospitalization rate of a patient population and/or a decrease in hospitalization length for a patient population.

A “pharmaceutical composition” refers to a mixture of the compounds described herein or pharmaceutically acceptable salts, esters, or prodrugs thereof, with other chemical components, such as physiologically acceptable carriers and excipients. One purpose of a pharmaceutical composition is to facilitate the administration of at least one gold(I) complex to a subject. A pharmaceutical composition may be formulated to contain a daily dose, or convenient fraction of a daily dose, in a dosage unit. In general, the pharmaceutical composition is prepared according to methods in pharmaceutical chemistry.

“Pharmaceutically acceptable salt” or “pharmaceutically acceptable ester” refers to a compound in a pharmaceutically acceptable form such as an ester, a phosphate ester, a salt of an ester, or a related) which, upon administration to a subject in need thereof, provides at least one of the gold(I) complexes deserved herein. Pharmaceutically acceptable salts and esters retain the biological effectiveness and properties of the free bases, which are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like. Suitable salts include those derived from alkali metals, such as potassium and sodium, and alkaline earth metals, such as calcium and magnesium, among other acids well-known in the art.

As used herein, a “pharmaceutically acceptable carrier” refers to a carrier or vehicle which may comprise an excipient, diluent, or mixture thereof that does not cause irritation to an organism and does not abrogate the biological activity and properties of the administered gold(I) complex. The term carrier encompasses any excipient, diluent, filler, salt, buffer, stabilizer, solubilizer, lipid, stabilizer, or other material well known in the art for use in pharmaceutical formulations. Suitable formulations may be prepared by methods commonly employed using conventional, organic and inorganic additives, such as an excipient selected from fillers or diluents, binders, disintegrants, lubricants, flavoring agents, preservatives, stabilizers, suspending agents, dispersing agents, surfactants, antioxidants, solubilizers, and the like. The choice of a carrier for use in a composition will depend upon the intended route of administration for the composition. The preparation of pharmaceutically acceptable carriers and formulations containing these materials is described in, for example, Remington's Pharmaceutical Sciences, 21st Edition, ed. University of the Sciences in Philadelphia, Lippincott, Williams & Wilkins, Philadelphia Pa., 2005, which is incorporated herein by reference in its entirety. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

An “excipient” refers to an inert substance added to a pharmaceutical composition to facilitate the administration of a compound further. Examples, without limitation, of excipients include filler or diluents (e.g., sucrose, starch, mannitol, lactose, glucose, cellulose, talc, calcium carbonate, calcium phosphate, and the like), a binder (e.g., cellulose, carboxymethylcellulose, methylcellulose, hydroxy methylcellulose, hydroxypropyl methylcellulose, polypropylpyrrolidone, polyvinylpyrrolidone, gelatin, gum Arabic, polyethylene glycol, starch, and the like), a disintegrants (e.g., sodium starch glycolate, croscarmellose sodium, and the like), a lubricant (e.g., magnesium stearate, light anhydrous silicic acid, talc, sodium lauryl sulfate, and the like), a flavoring agent (e.g., citric acid, menthol, and the like), a preservative (e.g., sodium benzoate, sodium bisulfite, methylparaben, propylparaben, and the like), a stabilizer (e.g., citric acid, sodium citrate, acetic acid, and the like), a suspending agent (e.g., methylcellulose, polyvinyl pyrrolidone, aluminum stearate, and the like), a dispersing agent (e.g., hydroxypropyl methylcellulose and the like), surfactants (e.g., sodium lauryl sulfate, polaxamer, polysorbates, and the like), antioxidants (e.g., ethylene diamine tetraacetic acid (EDTA), butylated hydroxyl toluene (BHT), and the like) and solubilizers (e.g., polyethylene glycols and the like).

Aspects of this disclosure are directed to the development of gold (I) complexes (also referred to as a complex) including 1,3-bis(2,6-diisopropylphenyl)imidazol-2-ylidene (IPr) and phosphane ligands. The complex was synthesized and evaluated for antitumor activity in ovarian cancer (OvCa) models. The synthesized complexes were characterized by Fourier transform infrared (FTIR), mass spectrometry (MS), nuclear magnetic resonance (NMR) spectroscopy, and X-ray diffractogram (XRD) crystallography. The antiproliferative effect of the complexes of the present disclosure was found to be higher than cisplatin and auranofin in OvCa cells sensitive and resistant to cisplatin.

A gold complex for treating cancer is described. The complex includes gold or precursors thereof. Suitable examples of gold precursors include, chloro(triethylphosphine)-gold(I), chloro(trimethylphosphine)gold(I), chloro[diphenyl(o-tolyl)phosphine]gold(I), chloro[tri(o-tolyl)phosphine]gold(I), chloro(methyldiphenylphosphine)gold(I), chloro[2-(dicyclohexyl phosphino)-biphenyl]gold(I), chloro[2-di-tert-butyl(2′,4′,6′-triisopropylbiphenyl)phosphine]gold(I), chloro[di(1-adamantyl)-2-dimethylaminophenylphosphine]gold(I), chloro(2-dicyclo hexyl-phosphino-2′-dimethylaminobiphenyl)gold(I), chloro(trimethyl phosphite)gold(I), chloro[(1,1′-biphenyl-2-yl)di-tert-butylphosphine]gold(I), chloro[2-dicyclohexyl(2′,4′,6′-trisopropyl-biphenyl)phosphine]gold(I), chloro[tris(2,3,4,5,6-pentafluorophenyl)-phosphine]gold(I), chloro[tri(p-tolyl)phosphine]gold(I), chloro[2-dicyclohexyl(2′,6′-dimethoxybiphenyl)-phosphine]gold(I), chloro[2-dicyclohexyl(2′,6′;-diisopropoxybiphenyl)-phosphine]gold(I), chloro[2-dicyclohexylphosphino-2′,6′-bis(N,N-dimethylamino)-biphenyl]gold(I), chloro {4-[2-di(1-adamantyl)phosphino]phenylmorpholine}gold(I), chloro(2-di-tert-butylphosphino-3,6-dimethoxy-2′,4′,6′-triisopropylbiphenyl)gold(I), chloro[2-(dicyclohexylphosphino)-3,6-dimethoxy-2′,4′,6′-triisopropyl-1,1′-biphenyl]gold(I), chloro(2-{bis[3,5-bis(trifluoromethyl)-phenyl]phosphino}-3,6-dimethoxy-2′,4′,6′-triisopropyl-1,1′-biphenyl)gold(I), and chloro(2-di-tert-butylphosphino-3,4,5,6-tetramethyl-2′,4′,6′-triisopropylbiphenyl)gold(I). In a preferred embodiment, the gold precursor is chloro[1,3-bis(2,6-diisopropylphenyl)imidazole-2-ylidene]gold(I).

The complex further includes two or more ligands, with each ligand that is coordinated, preferably chelated to the gold. The ligands are based on a phosphane ligand and an imidazole-2-ylidene ligand. The imidazole-2-ylidene-based ligand is a 1,3-bis(2,6-di-isopropyl phenyl)imidazole-2-ylidene. The gold complex of the present disclosure is mononuclear with one gold atom. The gold atom is bonded to both ligands—namely, the phosphane ligand and the imidazole-2-ylidene ligand. The gold atom is bonded to a first nucleophilic carbon atom of an N-heterocyclic carbene (NHC) of the imidazole-2-ylidene ligand and a first phosphorous atom of the phosphane ligand. In a preferred embodiment the first nucleophilic carbon atom of the NHC of the imidazole-2-ylidene ligand, the gold atom, and the first phosphorous atom of the phosphane ligand are bound in a near linear geometry with a bond angle from 177 to 180°. In an embodiment, the complex may have one or more imidazole-2-ylidene functional groups.

The phosphane ligand can be either monodentate (i.e., monophosphane and having one phosphorous donor atom) or bidentate (i.e., bisphosphane and having two phosphorous donor atoms) and include derivatives thereof. Suitable examples of the phosphane ligand include bis(2-cyanoethyl)phenylphosphane, (1R,2R)-2-diphenylphosphano)-1-aminocyclohexane, (1R,2R)-2-diphenylphosphano)-1,2-diphenylethylamine, (R)-2-(diphenylphosphano)-1-phenylethylamine, (R)-1-(diphenylphosphano)-2-amino-3,3-dimethylbutane, tricyclohexylphosphane, and 3-(diphenylphosphano)propylamine). In a preferred embodiment, the phosphane ligand is tricyclohexylphosphane.

The complex of the present disclosure has a half maximal inhibitory concentration of 0.1 to 2.5 μM, in a first ovarian cancer cell line, A2780, and a half maximal inhibitory concentration of 0.1 to 4.0 μM, preferably 0.10 to 0.15 μM, in a first ovarian cancer cell line resistant to cisplatin, A2780cis. The half maximal inhibitory concentration values change based on the choice of the phosphane ligand. For example, when the phosphane ligand in the complex is tricyclohexylphosphene, the complex has a half maximal inhibitory concentration of 0.10 to 0.20 μM in the first ovarian cancer cell line, A2780, and 0.10 to 0.15 μM in the first ovarian cancer cell line resistant to cisplatin, A2780cis. In some embodiments, the complex has a half maximal inhibitory concentration of 0.55 to 0.65 μM in a second ovarian cancer cell line, SKOV3, when the phosphane ligand is tricyclohexylphosphene.

In some embodiments, the complex has a fold resistance of 0.5 to 2. The fold resistance is based on a ratio of a half maximal inhibitory concentration in the first ovarian cancer cell line resistant to cisplatin, A2780cis, to a half maximal inhibitory concentration in the first ovarian cancer cell line, A2780. The fold resistance values change based on the choice of the phosphane ligand. For example, when the phosphane ligand in the complex is tricyclohexylphosphene, the complex has a fold resistance of less than 1.

In certain embodiments, the complex can further include a counter-anion to form a pharmaceutically acceptable salt. Non-limiting examples of counter-anions include halides such as fluoride, chloride, bromide, iodide, nitrate, sulfate, phosphate, methane sulfonate, ethane sulfonate, p-toluenesulfonate, salicylate, malate, maleate, succinate, tartrate, citrate, acetate, perchlorate, trifluoromethanesulfonate (triflate), acetylacetonate, hexafluorophosphate, and hexafluoroacetylacetonate. In some embodiments, the counter-anion is a hexafluorophosphate.

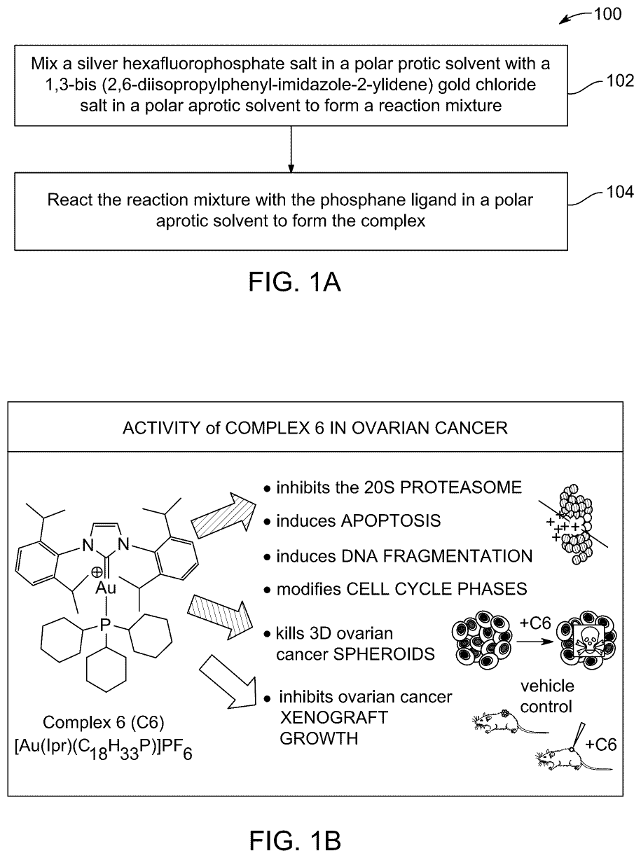

FIG. 1A illustrates a flow chart of a method 100 of preparing the complex. The order in which the method 100 is described is not intended to be construed as a limitation, and any number of the described method steps can be combined in any order to implement the method 100.

Additionally, individual steps may be removed or skipped from the method 100 without departing from the spirit and scope of the present disclosure.

At step 102, the method 100 includes mixing a silver hexafluorophosphate salt in a polar protic solvent with a 1,3-bis(2,6-diisopropylphenyl-imidazole-2-ylidene) gold chloride salt in a polar aprotic solvent to form a reaction mixture. The silver hexafluorophosphate salt and the 1,3-bis(2,6-diisopropylphenyl-imidazole-2-ylidene) gold chloride salt are mixed in a molar ratio of 1:5 to 5:1, preferably 4:1 to 1:4, preferably 3:1 to 1:3, preferably 2:1 to 1:2, and more preferably about 1:1. Suitable examples of polar protic solvents include, alcohol, water, hydrogen fluoride, formic acid, acetic acid, ammonia, and the like. The alcohol may be ethanol, methanol, isopropanol, and the like. In a preferred embodiment, the polar protic solvent is ethanol. Suitable examples of polar aprotic solvents include, for example, DMF (dimethylformamide), DMPU (dimethyl pyrimidinone), DMSO (dimethyl sulfoxide), DMA (dimethylacetamide), NMP (N-methylpyrrolidone), DMAC (dimethyl acetamide), tetrahydrofuran (THF), acetonitrile, acetone, dichloromethane the like, and combinations thereof. In a preferred embodiment, the polar aprotic solvent is acetone. The mixing is carried out at a temperature range of 20-35° C., preferably 20-25° C., and more preferably about 20-22° C., for a period of 1-30 minutes, preferably 2-25 minutes, preferably 5-20 minutes, and more preferably about 10-15 minutes. The reaction mixture may be further filtered to obtain a filtrate. The reaction mixture may be filtered via gravity filtration, vacuum filtration, pressure filtration, and the like through a filter paper, a glass frit, a column, and the like.

At step 104, the method 100 includes reacting the reaction mixture with the phosphane ligand in a polar aprotic solvent to form the complex. Suitable examples of phosphane ligand include bis(2-cyanoethyl) phenylphosphane, (1R,2R)-2-diphenylphosphano)-1-aminocyclohexane, (1R,2R)-2-diphenylphosphano)-1,2-diphenylethylamine, (R)-2-(diphenylphosphano)-1-phenylethylamine, (R)-1-(diphenylphosphano)-2-amino-3,3-dimethylbutane, tricyclohexylphosphane, and 3-(diphenylphosphano)propylamine). In a preferred embodiment, the phosphane ligand is tricyclohexylphosphane. Suitable examples of polar aprotic solvents include, for example, DMF, DMPU, DMSO, DMA, NMP, DMAC, THF, acetonitrile, acetone, dichloromethane, and the like, and combinations thereof. In a preferred embodiment, the polar aprotic solvent is dichloromethane. In some embodiments, the molar ratio of the phosphane ligand to a total molar amount the reaction mixture are in a ratio range of 1:5 to 5:1, preferably 4:1 to 1:4, preferably 3:1 to 1:3, preferably 2:1 to 1:2, and more preferably about 1:2. In an embodiment, the silver hexafluorophosphate salt, the 1,3-bis(2,6-diisopropylphenyl-imidazole-2-ylidene) gold chloride salt, and the phosphane ligands are in an equimolar amount, preferably about 1:1:1. The silver hexafluorophosphate salt, the 1,3-bis(2,6-diisopropylphenyl-imidazole-2-ylidene) gold chloride salt, and the phosphane ligands are allowed to react for a period of 10-60 minutes, preferably 20-40 minutes, preferably 20-30 minutes, and more preferably about 30 minutes, and filtered to obtain a crude product. The crude product is capped and left undisturbed for a period of 1-10 days, preferably 3-5 days. The crude product is further purified by various purification techniques conventionally known in the art (e.g., crystallization, recrystallization, filtration, distillation, and the like) to form the complex.

Another aspect of the present disclosure relates to a pharmaceutical composition comprising one or more of the complexes described herein. The complex described herein, or analogues or derivatives thereof, can be provided in a pharmaceutical composition. Depending on the intended mode of administration, the pharmaceutical composition can be in the form of solid, semi-solid, or liquid dosage forms, such as, for example, tablets, suppositories, pills, capsules, powders, liquids, or suspensions, preferably in a unit dosage form suitable for single administration of a precise dosage. The compositions will include a therapeutically effective amount of one or more of the gold(I) complexes described herein or derivatives thereof in combination with a pharmaceutically acceptable carrier and, in addition, may include other medicinal agents, pharmaceutical agents, carriers, diluents, excipients, or other non-active ingredients. By pharmaceutically acceptable, it is meant a material that is not biologically or otherwise undesirable, which can be administered to an individual along with the selected compound without causing significant unacceptable biological effects or interacting in a deleterious manner with the other components of the pharmaceutical composition in which it is contained.

The complex or an analog or derivative thereof may be used in conjunction with one or more additional compounds, in the treatment or prevention of neoplasm, of tumor or cancer cell division, growth, proliferation, and/or metastasis in a mammalian subject, inhibition of thioredoxin reductase (TrxR) activity/proteosome activity in tumor and/or cancer cells, induction of death or apoptosis of tumor and/or cancer cells, and/or any other form of a proliferative disorder. The complex of the present disclosure can be formulated as a pharmaceutical composition. In some embodiments, the pharmaceutical composition, including the complex or an analog or derivative thereof, is in an amount effective for treating a patient having ovarian cancer, and at least one pharmaceutical additive or adjuvant.

Thiol/selenol-containing proteins, like thioredoxin reductase (TrxR), are targets for gold(I) based anticancer agents. A mechanism of action is that phosphine gold(I) complexes act as irreversible inhibitors of at least the mammalian mitochondrial thioredoxin reductase (TrxR2), whose expression is elevated in cancer cells, thereby leading to the eventual death of the cancer cells [J. C. Lima and L. Rodriguez, J. Med. Chem., 11 (2011) 921; S. Urig, K. Fritz-Wolf, R. Reau, C. Herold-Mende, K. T6th, E. Davioud-Charvet and K. Becker, Angew. Chem. Int. Ed. 45 (2006) 1881; V. Gandin, A. P. Fernandes, M. P. Rigobello, B. Dani, F. Sorrentino, F. Tisato, M.

Bjornstedt, A. Bindoli, A. Sturaro, R. Relia and C. Marzano, Biochem Pharmacol 79 (2010) 90; and, Omata, Yo; Folan, Matt; Shaw, Melissa; Messer, Regina L.; Lockwood, Petra E.; Hobbs, David; Bouillaguet, Serge; Sano, Hidehiko; Lewis, Jill B.; Wataha, John C, Toxicology in vitro 20 (2006) 882, each incorporated herein by reference in its entirety].

The neoplastic activity of the tumor or cancer cells may be localized or initiated in one or more of the following: blood, brain, bladder, lung, cervix, ovary, colon, rectum, pancreas, skin, prostate gland, stomach, breast, liver, spleen, kidney, head, neck, testicle, bone (including bone marrow), thyroid gland, central nervous system. The complex of the present disclosure or the pharmaceutical composition thereof is effective in the treatment or prevention of ovarian cancer.

A pharmaceutical composition including the complex of the present disclosure can then be administered orally, systemically, parenterally, by inhalation spray, rectally, or topically in dosage unit formulations containing conventional nontoxic pharmaceutically acceptable carriers, adjuvants, and vehicles as desired. In some embodiments, the method of administration of the steroid or an analogue or derivative thereof is oral. In other embodiments, the compound or an analogue or derivative thereof is administered by injection, such as, for example, through a peritumoral injection.

Topical administration can also involve the use of transdermal administration, such as transdermal patches or iontophoresis devices. The term parenteral, as used herein, includes intravesical, intradermal, transdermal, subcutaneous, intramuscular, intralesional, intracranial, intrapulmonary, intracardial, intrasternal, and sublingual injections, or infusion techniques.

Formulation of drugs is discussed in, for example, Hoover, John E., Remington's Pharmaceutical Sciences, Mack Publishing Co., Easton, Pa.; 1975. Another example includes Liberman, H. A. and Lachman, L., Eds., Pharmaceutical Dosage Forms, Marcel Decker, New York, N.Y., 1980, which is incorporated herein by reference in its entirety.

Injectable preparations, for example, sterile injectable aqueous or oleaginous suspensions, can be formulated according to the known art using suitable dispersing or wetting agents and suspending agents. The sterile injectable preparation can also be a sterile injectable solution or suspension in a nontoxic, parenterally acceptable diluent or solvent, for example, as a solution in 1,3-butanediol. Among the acceptable vehicles and solvents that can be employed are water, Ringer's solution, isotonic sodium chloride solution, and the like. In addition, sterile, fixed oils are conventionally employed as a solvent or suspending medium. For this purpose, any fixed oil can be employed, including synthetic mono- or diglycerides. In addition, fatty acids such as oleic acid find use in the preparation of injectables. Dimethyl acetamide, surfactants including ionic and non-ionic detergents, and polyethylene glycols can be used. Mixtures of solvents and wetting agents, such as those discussed above, are also useful. Suppositories for rectal administration of the compound or an analogue or derivative thereof can be prepared by mixing the steroid or an analogue or derivative thereof with a suitable non-irritating excipient such as cocoa butter, synthetic mono- di- or triglycerides, fatty acids, and polyethylene glycols that are solid at ordinary temperatures but liquid at the rectal temperature and will therefore melt in the rectum and release the drug.

Solid dosage forms for oral administration can include capsules, tablets, pills, powders, and granules. In such solid dosage forms, the compounds of this disclosure are ordinarily combined with one or more adjuvants appropriate to the indicated route of administration. If administered orally, a contemplated steroid or an analogue or derivative thereof can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation, as can be provided in a dispersion of the active compound in hydroxypropyl methylcellulose. In the case of capsules, tablets, and pills, the dosage forms can also comprise buffering agents such as sodium citrate, magnesium or calcium carbonate, or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. A contemplated steroid or an analogue or derivative thereof of the present disclosure can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending, agents, and sweetening, flavoring, and perfuming agents. The amount of active ingredient that can be combined with the carrier materials to produce a single dosage form varies depending upon the mammalian subject treated and the mode of administration.

Cancers such as, but not limited to, sarcomas, carcinomas, melanomas, myelomas, gliomas, and lymphomas can be treated or prevented with the complex provided herein. In some embodiments, a pharmaceutical composition incorporating the complex of the present disclosure is present in an amount effective for treating a patient having a proliferative disorder selected from the group consisting of head and neck cancer, breast cancer, lung cancer, colon cancer, prostate cancer, gliomas, glioblastoma, astrocytomas, glioblastoma multiforme, Bannayan-Zonana syndrome, Cowden disease, Lhermitte-Duclos disease, inflammatory breast cancer, Wilm's tumor, Ewing's sarcoma, Rhabdomyosarcoma, ependymoma, medulloblastoma, kidney cancer, liver cancer, melanoma, pancreatic cancer, sarcoma, osteosarcoma, giant cell tumor of bone, thyroid cancer, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, Hodgkin's lymphoma, non-Hodgkin's lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), testicular cancer, and the like. In some embodiments, the cancer is preferably one of breast cancer, adenocarcinoma, breast adenocarcinoma, colon cancer, colorectal cancer, lung cancer, prostate cancer, kidney cancer, liver cancer, melanoma, pancreatic cancer, renal cancer, hepatocellular cancer, cervical cancer, ovarian cancer, testicular cancer, and the like. In some embodiments, the pharmaceutical composition including the complex is effective in the treatment or prevention of ovarian cancer.

The methods for treating cancer and other proliferative disorders described herein inhibit, remove, eradicate, reduce, regress, diminish, arrest, or stabilize a cancerous tumor, including at least one of the tumor growth, tumor cell viability, tumor cell division and proliferation, tumor metabolism, blood flow to the tumor and metastasis of the tumor. In some embodiments, after treatment with one or more gold(I) complexes or a pharmaceutical composition thereof, the size of a tumor, whether by volume, weight, or diameter, is reduced by at least 5%, 10%, 15%, 20%, 25%, 30%, 40%, 50%, 60%, 70%, 75%, 80%, 85%, 90%, 95%, 99%, or 100%, relative to the tumor size before treatment. In other embodiments, after treatment with one or more gold(I) complexes of a pharmaceutical composition thereof, the size of a tumor does not reduce but is maintained the same as the tumor size before treatment. Methods of assessing tumor size include but are not limited to CT scan, MRI, DCE-MRI, and PET Scan.

In some embodiments, the method for treating cancer and other proliferative disorders involves the administration of a unit dosage or a therapeutically effective amount of one or more gold(I) complexes or a pharmaceutical composition thereof to a mammalian subject (preferably a human subject) or a patient in need thereof. As used herein, “a subject in need thereof” refers to a mammalian subject, preferably a human subject, who has been diagnosed with, is suspected of having, is susceptible to, is genetically predisposed to, or is at risk of having at least one form of cancer. During administration, the complex, preferably including tricyclohexylphosphane as the ligand, is contacted with an ovarian cancer in the form of at least one of a monolayer of cells, a multicellular tumor spheroid (MCTS), and an in vivo xenograft tumor, preferably for 12 hours to 10 days, preferably for about 24 hours, preferably for about 48 hours, preferably for about 72 hours, and preferably for 4-7 days. In a preferred embodiment, the cell line is A2780, A2780cis, or SKOV3. The cancer cell line is cultured in RPMI-1640 medium containing 5 to 10% by volume of a fetal bovine serum (FBS) based on a total volume at 37° C. in a 5% CO₂ humidified atmosphere with 0.5-2% by volume of penicillin-streptomycin and 0.5-2% by volume of L-glutamine. On administration, the complex induces apoptosis in A2780, A2780cis, and a multicellular tumor spheroid (MCTS) sample of the second ovarian cancer cell line, SKOV3.

In an embodiment, the complex administered at a concentration of 1 μM decreases the volume of SKOV3-MCTSs by 85 to 95% based on volume after 7 days. In an embodiment, the complex administered at a concentration of 0.50 μM generates mitochondrial reactive oxygen species (mitROS) in SKOV3-MCTSs of 50 to 60% based on a cell count after 24 hours. In an embodiment, the complex administered to a sample at a concentration of 0.1 to 0.6 μM has a number of cells in the S phase from 15 to 35% compared to a sample in the absence of the complex having an amount of cells in the S phase from 40 to 50% based on a cell count after a time of 24 hours. In an embodiment, the complex administered to a sample at a concentration of 0.1 to 0.6 μM has a number of cells in the G1 phase from 60 to 80% compared to a sample in the absence of the complex having a number of cells in the G1 phase from 45 to 55% based on a cell count after a time of 24 hours. In an embodiment, the complex administered to a sample at a concentration of 0.5 μM decreases proteasome activity by 30 to 70% after 24 hours based on a 20S-proteasome assay compared to a sample in the absence of the complex. In an embodiment, a sample is an in vivo xenograft tumor and the complex administered to the sample showed a reduction of the sample of more than 80% based on sample volume after 5 weeks of treatment of 2 milligrams of the complex per kilogram of a body weight of a host of the sample two times a week (FIG. 1).

The dosage and treatment duration are dependent on factors such as the bioavailability of a drug, administration mode, toxicity of a drug, gender, age, lifestyle, body weight, the use of other drugs and dietary supplements, cancer stage, tolerance, and resistance of the body to the administered drug, and the like, then determined and adjusted accordingly. One or more gold(I) complexes or a pharmaceutical composition thereof may be administered in a single dose or multiple individual divided doses. In some embodiments, the interval of time between the administration of complex or a pharmaceutical composition thereof and the administration of one or more additional therapies may be about 1-5 minutes, 1-30 minutes, 30-60 minutes, 90 minutes, 1-2 hours, 2-6 hours, 2-12 hours, 12-24 hours, 1-2 days, 2 days, 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 15 weeks, 20 weeks, 26 weeks, 52 weeks, 11-15 weeks. 15-20 weeks, 20-30 weeks, 30-40 weeks, 40-50 weeks, 1 month, 2 months, 3 months, 4 months 5 months, 6 months, 7 months, 8 months, 9 months, 10 months, 11 months, 12 months, 1 year, 2 years, 5 years, 10 years, indefinitely, or any period in between. In certain embodiments, the complex and one or more additional therapies are administered less than 1 day, 1 week, 2 weeks, 3 weeks, 4 weeks, 1 month, 2 months, 3 months, 6 months, 1 year, 2 years, or 5 years apart.

In certain embodiments, the complex of the present disclosure or a pharmaceutical composition thereof may be used in combination with one or more other antineoplastic or chemotherapeutic agents. A non-limiting list of examples of chemotherapeutic agents are aflibercept, asparaginase, bleomycin, busulfan, carmustine, chlorambucil, cladribine, cyclophosphamide, cytarabine, dacarbazine, daunorubicin, doxorubicin, etoposide, fludarabine, gemcitabine, hydroxyurea, idarubicin, ifosamide, irinotecan, lomustine, mechclorethamine, melphalan, mercaptopurine, methotrexate, mitomycin, mitoxantrone, pentostatin, procarbazine, 6-thioguanine, topotecan, vinblastine, vincristine, retinoic acid, oxaliplatin, cis-platin, carboplatin, 5-FU (5-fluorouracil), teniposide, amasacrine, docetaxel, paclitaxel, vinorelbine, bortezomib, clofarabine, capecitabine, actinomycin D, epirubicine, vindesine, methotrexate, tioguanine (6-thioguaniue), tipifarnib. Examples for antineoplastic agents which are protein kinase inhibitors include imatinib, erlotinib, sorafenib, sunitinib, dasatinib, nilotinib, lapatinib, gefitinib, temsirolimus, everolimus, rapamycine, bosutinib, pzopanib, axitinib, neratinib, vatalanib, pazopanib, midostaurin and enzastaurin. Examples for antineoplastic agents which are antibodies comprise trastuzumab, cetuximab, panitumumab, rituximab, bevacizumab, mapatumumab, conatumumab, lexatumumab and the like.

### Examples

The disclosure will now be illustrated with working examples, which are intended to illustrate the working of disclosure and not intended to restrictively imply any limitations on the scope of the present disclosure, as many variations thereof are possible without departing from the spirit and scope of the present disclosure. The working examples depict an example of the method of the present disclosure.

### Materials

All solvents were of analytical grade and were used without further purification. Ethanol, diethyl ether, dichloromethane, and acetone were purchased from Fluka AG (St. Gallen, Switzerland). Chlorido[1,3-bis(2,6-diisopropylphenyl)-imidazol-2-ylidene]gold(I) (Au(IPr)Cl), bis(2-cyanoethyl)-phenylphosphane, (1R,2R)-2-(diphenylphosphano)-1-aminocyclohexane, (1R,2R)-2-(diphenylphosphano)-1,2-diphenylethylamine, (R)-2-(diphenylphosphano)-1-phenylethylamine, (R)-1-(diphenylphosphano)-2-amino-3,3-dimethylbutane, tricyclohexylphosphane, and 3-(diphenylphosphano)propylamine were purchased from Strem Chemicals Inc. (Newburyport, Massachusetts, United States). AgPF₆ was purchased from Sigma-Aldrich.

### Instrumentation

¹H, ¹³C, and ³¹P Nuclear Magnetic Resonance (NMR) spectra were recorded on a JEOL-LA 500 NMR spectrophotometer, operating at 500.0, 125.65, and 202 MHz, respectively, corresponding to a magnetic field of 11.74 T. The spectral conditions for ¹H NMR included the following: 32K data points, 3.2 second acquisition time, and a 5.75 s pulse width. ¹³C NMR spectra were obtained with ¹H broadband decoupling and the following spectral conditions: 32K data points, 1 second acquisition time, 2.5 second pulse delay, and a 5.12 μs pulse width. All spectra were recorded at 297 K in CDCl₃ using tetramethylsilane (TMS) as an internal standard. Phosphoric acid was used as an external standard for ³¹P NMR at 0.0 ppm.

High-resolution electrospray ionization (ESI) mass spectrometry analysis of the seven (1-7) gold complexes was performed by Orbitrap Qexactive Plus mass spectrometer (Thermo Scientific). Samples, dissolved in water/acetonitrile (1:1) solution at a concentration of 1 μM, were directly infused through the ionization source (heated-ESI, HESI) into the Orbitrap mass analyzer operated in positive ionization mode with a source voltage 2.0 kV, capillary temperature 320° C., and source heater temperature 50° C. Data were acquired at resolution 70,000 with a Fourier transform (FT)-MS instrument in a range of mass-to-charge ratios (m z) of 500-1000. Elemental analyses were obtained on PerkinElmer series 11 (CHNS/O) analyzer 2400. The solid-state FTIR spectra of the free ligands and their gold(I) complexes were recorded on a PerkinElmer FTIR 180 spectrophotometer, using KBr pellets over the range 400-4000 cm⁻¹ at resolution 4 cm⁻.

Synthesis of Complex [Au(IPr)(C₁₂H₁₃N₂P)] PF₆ (1)

Complex 1 was prepared by adding AgPF₆ (0.127 g, 0.500 mmol) in 5.0 mL of ethanol to a solution of [Au(IPr)Cl] (0.311 g, 0.500 mmol) in 5.0 mL of acetone. The resulting mixture was stirred at ambient temperature for 10 minutes, and then it was filtered off. A solution of bis(2-cyanoethyl)-phenylphosphane (0.108 g, 0.500 mmol) in 5.0 mL of CH₂C₂ was added to the filtrate, stirred for 30 minutes, filtered off, and stored in an undisturbed area. After 5 days, colorless crystals were obtained, washed with diethyl ether (5.0 mL×3), and recrystallized from the mixture of acetone/CH₂Cl₂ (1:1). A suitable crystal was selected for X-ray diffraction analysis. Yield: 0.313 g (66%). The monoisotopic m/z was 801.3354, differing −0.10×10⁻³ atomic mass unit (amu) from exact mass=801.3355. Anal. Calcd for C₃₉H₄₉AuN₄P₂F₆ (946.75 g/mol): C, 49.42; H, 5.31; N, 5.91. Found: C, 49.30; H, 5.28; N, 6.01. IR (cm⁻): 3120 v(═C—H) sym, 1256 v(═C—H) bend, 2956 v(CH₂) asym, 2874 v(CH₂) sym, 1467 v(C═C), 2242 v(C≡C), 1327 v(C—H), 1108 v(C—P).

Synthesis of Complex [Au(IPr)(C₁₈H₂₂NP)] PF₆ (2)

Complex 2 was prepared by adding AgPF₆ (0.127 g, 0.500 mmol) in 5.0 mL of ethanol to a solution of [Au(IPr)Cl] (0.311 g, 0.500 mmol) in 5.0 mL of acetone. The resulting mixture was stirred at ambient temperature for 15 minutes, and then the mixture was filtered off. A solution of (1R,2R)-2-(diphenylphosphano)-1-aminocyclohexane (0.142 g, 0.500 mmol) in 5 mL of CH₂C₂ was added to the filtrate. Then the mixture was stirred for 30 minutes, filtered off, and stored in an undisturbed area. After 3 days, a colorless solid was obtained, washed with diethyl ether (5.0 mL×3), and recrystallized from the mixture of acetone/CH₂Cl₂ (1:1). Yield: 0.344 g (68%). The monoisotopic m z was 868.4035, differing 0.7×10⁻³ amu from exact mass=868.4028. Anal. Calcd for C₄₅H₅₉AuN₃P₂F₆ (1014.87 g/mol): C, 53.25; H, 5.31; N, 4.14. Found: C, 53.61; H, 5.40; N, 4.15. IR (cm⁻): 3433, 3358 v(NH), 3174 v(═C—H) sym, 2963 v(CH₂) asym, 2867 v(CH₂) sym, 1478 v(C═C), 1327 v(C—H), 1215 v(C—N), 1103 v(C—P).

Synthesis of Complex [Au(IPr)(C₂₆H₂₄NP)] PF₆ (3)

Complex 3 was prepared by adding AgPF₆ (0.127 g, 0.500 mmol) in 5.0 mL of ethanol to a solution of [Au(IPr)Cl] (0.311 g, 0.500 mmol) in 5.0 mL of acetone. The resulting mixture was stirred at ambient temperature for 15 minutes, and then the mixture was filtered off. A solution of (1R,2R)-2-(diphenylphosphano)-1,2-diphenylethylamine (0.159 g, 0.500 mmol) in 5.0 mL of CH₂C₂ was added to the filtrate. Then the mixture was stirred for 30 minutes, filtered, and stored in an undisturbed area. After 5 days, a yellow solid was obtained, washed with diethyl ether (5.0 mL×3), and recrystallized from the mixture of acetone/CH₂Cl₂ (1:1). Yield: 0.363 g (65%). The monoisotopic m z was 966.4192, differing 0.7×10⁻³ amu from exact mass=966.4185. Anal. Calcd for C₅₃H₆₁AuN₃P₂F₆ (1112.98 g/mol): C, 57.19; H, 5.52; N, 3.77. Found: C, 56.88; H, 5.63; N, 3.79. IR (cm⁻): 3288, 3275 v(NH), 3171 v(═C—H) sym, 2960 v(CH₂) asym, 2869 v(CH₂) sym, 1469 v(C═C), 1329 v(C—H), 1214 v(C—N), 1118 v(C—P).

Synthesis of Complex [Au(IPr)(C₂₀H₂₀NP)] PF₆ (4)

Complex 4 was prepared by adding AgPF₆ (0.127 g, 0.500 mmol) in 5.0 mL of ethanol to a solution of [Au(IPr)Cl] (0.311 g, 0.500 mmol) in 5.0 mL of acetone. The resulting mixture was stirred at ambient temperature for 15 minutes, and then the mixture was filtered off. A solution of (R)-2-(diphenylphosphano)-1-phenylethylamine (0.153 g, 0.500 mmol) in 5.0 mL of CH₂C₂ was added to the filtrate. Then the mixture was stirred for 30 minutes, filtered, and stored in an undisturbed area. After 5 days, a yellow solid was obtained, washed with diethyl ether (5.0 mL×3), and recrystallized from the mixture of acetone/CH₂Cl₂ (1:1). Yield: 0.402 g (78%). The monoisotopic m z was 890.3876, differing 0.4×10⁻³ amu from exact mass=890.3872. Anal. Calcd for C₄₇H₅₆AuN₃P₂F₆ (1035.87 g/mol): C, 54.49; H, 5.54; N, 4.05. Found: C, 54.47; H, 5.66; N, 3.99. IR (cm⁻): 3400, 3392 v(NH), 3168 v(═C—H) sym, 2962 v(CH₂) asym, 2869 v(CH₂) sym, 1467 v(C═C), 1329 v(C—H), 1214 v(C—N), 1184 v(C—P).

Synthesis of Complex [Au(IPr)(C₁₈H₂₄NP)] PF₆ (5)

Complex 5 was prepared by adding AgPF₆ (0.127 g, 0.500 mmol) in 5.0 mL of ethanol to a solution of [Au(IPr)Cl] (0.311 g, 0.500 mmol) in 5.0 mL of acetone. The resulting mixture was stirred at ambient temperature for 10 minutes, and then the mixture was filtered off. A solution of (R)-1-(diphenylphosphano)-2-amino-3,3-dimethylbutane (0.143 g, 0.500 mmol) in 5.0 mL of CH₂C₁₂ was added to the filtrate. Then the mixture was stirred for 30 minutes, filtered off, and stored in an undisturbed area. After 4 days, a colorless solid was obtained, washed with diethyl ether (5.0 mL×3), and recrystallized from the mixture of acetone/CH₂Cl₂ (1:1). Yield: 0.357 g (77%). The monoisotopicm z was 870.4187, differing 0.2×10⁻³ amu from exact mass=870.4185. Anal. Calcd for C₃₉H₄₉AuN₃P₂F₆ (932.73 g/mol): C, 53.20; H, 5.95; N, 4.13. Found: C, 52.96; H, 5.84; N, 4.02. IR (cm⁻): 3292, 3285 v(NH), 3172 v(═C—H) sym, 2958 v(CH₂) asym, 2870 v(CH₂) sym, 1472 v(C═C), 1329 v(C—H), 1217 v(C—N), 1105 v(C—P).

Synthesis of Complex [Au(IPr)(C₁₈H₃₃P)] PF₆ (6)

Complex 6 was prepared using tricyclohexylphosphane (0.140 g, 0.50 mmol) by a procedure similar to 1. After 5 days, a colorless solid was obtained, washed with diethyl ether (5.0 mL×3), and recrystallized from the mixture of acetone/CHCl₃ (1:1). Yield: 0.411 g (81%). The monoisotopic m/z was 865.4852, differing −0.6×10⁻³ amu from exact mass=865.4858. Anal. Calcd for C₄₅H₆₉AuN₂P₂F₆.CHCl₃ (1130.29 g/mol): C, 48.88; H, 6.24; N, 2.48. Found: C, 48.49; H, 6.34; N, 2.55. IR (cm⁻): 3167 v(═C—H) sym 2927 v(CH₂) asym, 2853 v(CH₂) sym, 1452 v(C═C), 1328 v(C—H), 1272 v(C—N), 1119 v(C—P).

Synthesis of Complex [Au(IPr)(C₁₅H₁₈NP)] PF₆ (7).

Complex 7 was prepared by adding AgPF₆ (0.127 g, 0.500 mmol) in 5.0 mL of ethanol to a solution of [Au(IPr)Cl] (0.311 g, 0.500 mmol) in 5.0 mL of acetone. The resulting mixture was stirred at ambient temperature for 10 minutes, and then the mixture was filtered off. A solution of 3-(diphenylphosphano)propylamine (0.122 g, 0.5 mmol) in 5.0 mL of CH₂C₁₂ was added to the filtrate. Then the mixture was stirred for 30 minutes, filtered, and stored in an undisturbed area. After 3 days, a colorless solid was obtained, washed with diethyl ether (5.0 mL×3), and recrystallized from the mixture of acetone/CH₂Cl₂ (1:1). Yield: 0.310 g (64%). The monoisotopic m z was 828.3715, differing 0.0×10⁻³ amu from exact mass=828.3715. Anal. Calcd for C₄₂H₅₄AuN₃P₂F₆ (973.80 g/mol): C, 51.80; H, 5.58; N, 4.31. Found: C, 52.18; H, 5.48; N, 4.41. IR (cm⁻): 3392, 3326 v(NH), 3148 v(═C—H) sym, 2962 v(CH₂) asym, 2870 v(CH₂) sym, 1497 v(C═C), 1329 v(C—H), 1215 v(C—N), 1103 v(C—P). As indicated by the C, H, and N analysis in this section, the purity of all complexes was >95%.

### X-ray Structure Determination of Complex 6.

Suitable crystals of complex 6 were obtained as colorless blocks in a mixture of acetone/CHCl₃ (1:1) by slow evaporation at room temperature. The intensity data were collected at 203 K (−70° C.) on a Stoe Mark II image plate diffraction system equipped with a two-circle goniometer and using Mo Kα graphite monochromated radiation (λ=0.71073 Å). The structure was solved by direct methods with SHELXS-2014. The refinement and all further calculations were carried out with SHELXL-2014. The C-bound H-atoms were included in calculated positions and treated as riding atoms: C—H=0.94-0.99 Å with Uᵢₛₒ(H)=1.5U_{eq}(C) for methyl H atoms and =1.2U_{eq}(C) for other H-atoms. The non-H atoms were refined anisotropically, using weighted full-matrix least-squares on F². A semiempirical absorption correction was applied using the MULABS routine in PLATON. The figures were drawn using Mercury. The summary of crystal data and details of the structure refinement of complex 6 are shown in Table 1.

TABLE 1
Summary of crystal data and details of the structure
refinement for complex 6

| Complex 6 | Experimental details |
|---|---|
| Crystal data | |
| CCDC number | 2166156 |
| Empirical formula | C₄₅H₆₉AuN₂P•PF₆•CHCl₃ |
| Formula weight | 1130.29 |
| Wavelength (Å) | 0.71073 |
| Temperature (K) | 203 |
| Crystal symmetry | Orthorhombic |
| Crystal color | Plate, colorless |
| Crystal size/mm | 0.40 × 0.22 × 0.03 |
| Space group | Cmc2₁ |
| Cell lengths (Å) | a = 17.2904 (7), b = 18.2392 (11), c = 16.1154 (7) |
| Cell Angles (°) | α = 90.000 (5), β = 90.000 (5), γ = 90.000 (5) |
| Dₓ | 1.477 Mg m⁻³ |
| M | 3.17 mm⁻¹ |
| Radiation type | Mo Kα |
| Cell volume (Å³) | 5082.2(4) |
| Z | 4 |
| Data collection | |
| Diffractometer | STOE IPDS 2 |
| Absorbance correction | Multi-scan (MULABS; Spek, 2009) |
| Radiation source | fine-focus sealed tube |
| Radiation monochromator | plane graphite |
| Tₘᵢₙ, Tₘₐₓ | 0.746, 1.000 |
| (sin θ/λₘₐₓ (A^{o-1})) | 0.622 |
| Refinement | |
| R[F² > 2σF²)], wR(F²), S | 0.024, 0.047, 0.90 |
| No. of reflections | 5285 |
| No. of parameters | 290 |
| No. of restraints | 1 |
| Δρ_{maxy} | 0.95 e Å⁻³ |
| Δρₘᵢₙ | −0.61 e Å⁻³ |
| H-atom treatment | H atoms treated by a mixture of independent |
| | and constrained refinement |

### Cell Lines and Culture Conditions

Human epithelial ovarian cancer A2780 cell line and its cisplatin-resistant clone A2780cis were obtained from Sigma-Aldrich (Milano, Italy). SKOV3 (HTB-77) cells were from the American Type Culture Collection (ATCC). The two cell lines were selected to reflect the two different stages of OvCa and different sensitivity to cisplatin. A2780 cell lines were derived from the ovarian tumor tissue of an untreated patient, while SKOV3 was from the ascitic fluids (after metastasis). Cells were authenticated in the laboratory using PowerPlex 16 HS system (Promega) and GeneMapper ID version 3.2.1 to identify DNA short tandem repeats. Cell lines were cultured in RPMI-1640 medium containing 10% fetal bovine serum (FBS) (Gibco, Thermo Fisher Scientific, U.S.A), 1% (v/v) of penicillin (10,000 units/mL)-streptomycin (10 mg/mL), and 1% (v/v) L-glutamine (200 mM) (all from Sigma- Aldrich) at 37° C. in a 5% CO₂ fully humidified atmosphere. A2780cis cells were continuously exposed to 1 μM cisplatin to maintain cisplatin resistance. Adipose-derived stem cells (ADSCs) were purchased from Lonza and maintained in mesenchymal-stem-cell growth medium Bulletkit MSCGM (Lonza, Verviers, Belgium).

Auranofin was from Sigma (Milano). Cisplatin (Teva) was a surplus drug from the clinical pharmacy of CRO Aviano.

### Cell Viability Assay

4.0×10³/mL A2780, A2780cis, and SKOV3 cells were seeded in 96-well microplates in 100 μL of culture medium. Cells were allowed to adhere for 24 hours and then were treated in triplicate with increasing concentrations of gold compounds (0-1 μM). Cisplatin was included as a reference drug. Cell viability was evaluated using 3-(4,5-dimethylthiazol-2-yl)-2,5-diphenyltetrazolium bromide (MTT) assay (Sigma-Aldrich) after 72 hours of treatment. Half maximal inhibitory concentrations (IC₅₀) were calculated using the CalcuSyn software version 2 (Biosoft, Ferguson, MO, USA). Fold resistance was calculated as the ratio of the IC₅₀ of the A2780cis to that of the parental cell line A2780.

Alternatively, cell viability was assayed by crystal violet staining. Briefly, after drug treatment culture medium was removed, cells were washed and stained by adding crystal violet solution for 20 minutes. Then it was solubilized (1% SDS solution) and quantified using a microplate reader at 570 nm absorbance.

### SKOV3-MCTSs

To generate single spheroids with defined size, 1.0×10³/mL SKOV3 cells were seeded 4 days under nonadherent conditions in U-bottom 96-well plates coated twice with 20 mg/mL of poly(2-hydroxyethyl methacrylate) (poly-HEMA; Sigma) in 95% ethanol. Spheroid size was measured at days 0, 3, and 7 after drug treatment initiation using an inverted microscope (Eclipse TS/100, Nikon, Tokyo, Japan) with photomicrographic systems DS camera control unit DS-L2. Spheroid volumes were calculated using the formula (width²×length×3.14)/6.

To obtain multiple spheroids, 5.0×10⁴/mL SKOV3 cells were seeded in nonadherent conditions on poly-HEMA coated wells for 4 days. After drug treatment, viable cells were evaluated using PrestoBlue cell viability reagent (Thermo Fisher Scientific, Frederick, MD, USA) or alternatively trypsinized to a single cell suspension for further analysis.

### Flow Cytometry

2.0×10⁵/mL A2780 and A2780cis cells were seeded in 6-well plates in complete medium and treated with complex 6 (0.15 μM and 0.5 μM). Results were detected by flow cytometry on a BD FACSCanto II flow cytometer. Data were analyzed using BD FACSDiva version 8.0.1 software (BD Biosciences) unless otherwise indicated. To evaluate apoptosis, tumor cells were treated for 48 hours and then stained for 15 minutes with FITC annexin-V (Thermo Fisher Scientific) and 7AAD (BD Pharmingen). Activated caspase-3,7 was detected using the CaspaTag caspase-3,7 assay (Merck Millipore). For B-cell lymphoma/leukemia-2 (Bcl-2) and Bcl-2 associated x protein (Bax) evaluation, cells were fixed and permeabilized using FIX & PERM cell fixation and cell permeabilization kit (Life Technologies). For ROS generation, cells were stained with 5 μM MitoSOX red mitochondrial superoxide indicator (ThermoFisher Scientific) in a working solution for 30 minutes at 37° C. Cell cycle analysis was performed after 24 hours treatment with complex 6. Cells were harvested, fixed in cold 70% ethanol for 15 minutes, and stained with propidium iodide (PI) solution (50 g/mL PI, 0.1% NP-40, 100 g/mL PureLink RNase A, 0.1% sodium citrate) for 1 hour. The distribution of cells in different cell cycle phases was quantified using ModFit LT 4.0 software (Verity Software House, Topsham, ME, USA).

### Thioredoxin Reductase and 20S Proteasome Activity

To evaluate both the thioredoxin reductase (TrxR) and proteasome activity, cells were treated with complex 6 for 24 hours and then lysed in 50 mM Tris-HCl, pH 7.6, 0.1% Triton X-100, 0.9% NaCl. TrxR (EC 1.8.1.9) was assayed using thioredoxin reductase assay kit (Sigma-Aldrich). Enzyme activity was determined by reading the absorbance at 412 nm using a spectrophotometer (Biomate 3 Thermo Spectronic, Thermo Electonic Corporation, Monza, Italy).

The enzymatic activity was normalized to the protein concentration and determined using the Bio-Rad protein assay (protein assay dye reagent concentrate, Bio-Rad Laboratories, Segrate, Italy).

Proteasome activity (EC 3.4.25.1) was evaluated on the same cell lysates as TrxR using the 20S proteasome activity assay kit APT280 (Merck Millipore) and a computer-interfaced GeniusPlus microplate reader (Tecan Trading AG, Switzerland). The activity was normalized to the protein concentration and expressed as a percentage of control.

### Western Blot

Whole-cell lysates were prepared using cold RIPA buffer [150 mM NaCl, 50 mM Tris-HCl (pH 8), 0.1% SDS, 1% Igepal, and 0.5% desoxycholate sodium] containing a protease inhibitor cocktail (Roche Diagnostics S.p.a., Milan, Italy), phosphatase inhibitors 1 mM Na₃VO₄ and 1 mM NaF (Sigma-Aldrich). Protein concentrations were determined using the BioRad protein assay (Bio-Rad Laboratories, Inc., CA, USA). Equal amounts of proteins were mixed with Laemmli buffer, separated in 4-20% SDS-PAGE (Criterion Precast Gel, BioRad), and blotted onto a nitrocellulose membrane (Amersham, GE Healthcare). Membrane strips were blocked with EveryBilot blocking buffer (Bio-Rad) and incubated at 4° C. overnight with primary antibodies probed with the appropriate antibodies (Table 2) and developed using Immobilon Western chemioluminescent HRP substrate. Images were acquired using a ChemiDoc XRS system (Bio-Rad).

TABLE 2
Antibodies

| Antigen | Host, Clone, Conjugation | Vendor |
|---|---|---|
| Bax | Mouse, Clone 3/Bax (RUO) | BD Pharmingen |
| Bcl-2 | Mouse, Clone 124, FITC | DAKO |
| | | Cytomation |
| Mouse IgG | Goat, Polyclonal, PE | Jackson |
| | | ImmunoResearch |
| α-tubulin | Mouse, Clone B-5-1-2 | Sigma Aldrich |
| Vinculin | Goat, vinculin (N-19): sc7649 | Santa Cruz |
| Mouse IgG | Alexa 488 Fluor-conjugated anti- | Molecular Probes |
| | mouse secondary antibody | |
| Mouse IgG | Donkey, anti-Mouse IgG (H + L | Bethyl |
| | chain) A90-137P | Laboratories |
| Phospho- | Mouse, anti-phospho-Histone | Millipore |
| H2A.X | H2A.X (Ser139)(clone JBW301) | |
| Raf-B | Mouse, Raf-B (F-7): sc-5284 | Santa Cruz |
| PI3 | Rabbit mAb, PI3 Kinase p110α | Cell Signaling |
| | (C73F8) | |
| Cyclin D1 | Mouse mAb (DCS-6), Anti- | Calbiochem |
| | Cyclin D1 (Ab-3) | |
| Cdk2 | Mouse, Cdk2 (D-12): sc-6248 | Santa Cruz |
| Cyclin D3 | Mouse, Cyclin D3 (DCS22) | Cell Signaling |
| Cyclin E | Rabbit, Cyclin E (E-4): sc-377100 | Santa Cruz |
| Cdk4 | Rabbit, Cdk4 (C-22): sc-260 | Santa Cruz |
| Cdk6 | Mouse, Cdk6 (DCS-83): sc-53638 | Santa Cruz |
| Cdc25A | Rabbit, Cdc25A (144): sc-97 | Santa Cruz |
| E2F-1 | Rabbit, E2F-1 (C-20): sc-193 | Santa Cruz |
| Phospho- | Rabbit, Phospho-p44/42 MAPK | Cell Signaling |
| Erk1/2 | (Erk1/2) (Thr202/Tyr204) | |
| | Antibody | |
| Phospho- | Rabbit, Fox03a (75D8) | Cell Signaling |
| Fox03a | | |
| Phospho- | Rabbit, Phospho-GSK-3-beta | Cell Signaling |
| GSK-3-β | (Ser9) (D3A4) | |
| Phospho-Rb | Mouse, p-RB (Ser780): sc-12901 | Santa Cruz |

Cell migration was assessed using the in vitro wound healing assay. Briefly, cells were grown to confluence and then treated with complex 6. After 24 hours of treatment, the medium was removed and cells were washed then scraped with a pipet tip to create a “wound” in the monolayer and washed again. Fresh medium containing 2% FCS was added. Wounds were photographed at times 0, 24, and 48 hours using an inverted microscope (EclipseTS/100, Nikon, Instruments Europe BV Amsterdam, The Netherlands) at magnification 4. Migration was assessed by measuring the cell-free area (in pixels) with ImageJ-NIH (National Institutes of Health) tool software. The cytoskeleton organization was evaluated by culturing SKOV3 cells onto glass coverslips and performing immunofluorescence staining for α-tubulin and F-actin. SKOV3 cells were treated for 24 hours with complex 6, fixed in 4% paraformaldehyde, permeabilized with 0.5% Triton X-100, and incubated overnight with primary antibody (Table 1) at 4° C. Primary antibody was detected using the appropriate secondary antibody together with phallotoxins (Molecular Probes) and TO-PRO-3 (Thermo Fisher Scientific). Images were acquired using an SP8 confocal microscope (Leica).

### In Vivo Studies

Animal experimentation was reviewed and approved by Centro di Riferimento Oncologico di Aviano (CRO) Institutional Organism for Animal Wellbeing (OPBA) and approved by the Italian Ministry of Health (No. 671/2015/PR). All animal experiments were conducted in adherence to the international and institutional committees' ethical guidelines. Ten 4-week-old female athymic nude NU(NCr)-Foxn1^{nu} mice were purchased from Charles Rivers (Lecco, Italy). SKOV3 (10×10⁶ cells/animal) were suspended in BD Matrigel basement membrane matrix (BD Biosciences) (diluted 1:3 in PBS) and inoculated into the animals' right flank. When tumors were palpable, animals were divided into two groups of five mice each, and treated with intratumoral injection of complex 6 (2 mg/kg) or vehicle (10% DMSO, 20% Cremophor Sigma-Aldrich, 70% PBS) two times a week. Body weight and tumors were examined regularly. Tumor size was measured using a digital caliper, and volumes were calculated according to the formula (width²×length×3.14)/6.

### Statistical Analysis

Statistical analysis was carried out using GraphPad Prism version 6.0 software (GraphPad, La Jolla, USA). Student's t test was used to compare the two groups. One-way ANOVA followed by Dunnett's test was used to compare each of a number of treatments with a single control. A P-value of <0.05 was considered significant.

### Synthesis and Spectroscopic Characterization.

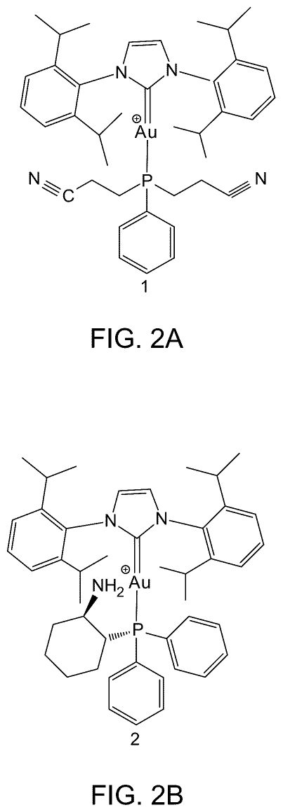

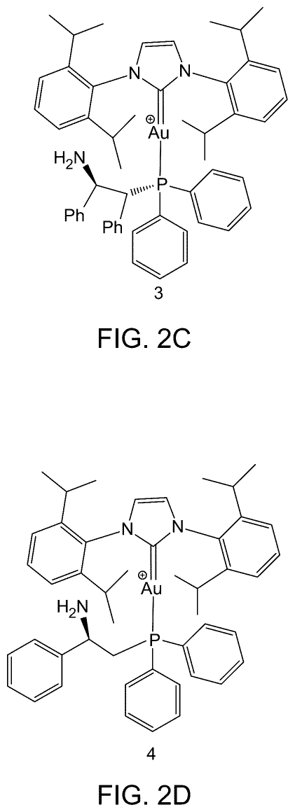

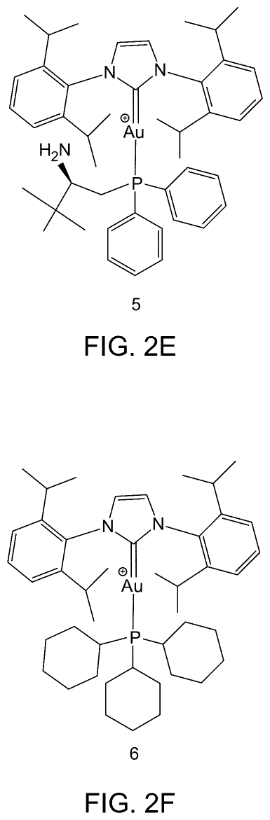

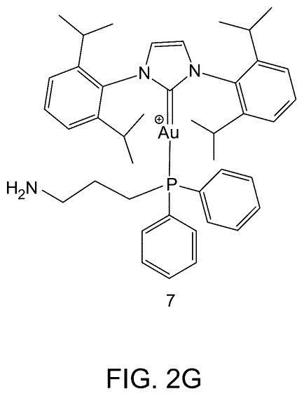

A series of seven gold(I) complexes 1-7 containing 1,3-bis(2,6-di-isopropylphenyl)imidazol-2-ylidene (IPr) and phosphane ligands (L1-L7), [Au(IPr)(L1)] PF₆ (1), [Au(IPr)(L2)] PF₆ (2), [Au(IPr)(L3)] PF₆ (3), [Au(IPr)(L4)] PF₆ (4), [Au(IPr)(L5)] PF₆ (5), [Au(IPr)(L6)] PF₆ (6), and [Au(IPr)(L7)] PF₆ (7), were synthesized and characterized by elemental analyses, mass spectrometry, mid-FTIR (Fourier transform infrared) spectroscopy, and ¹H, ¹³C, and ³¹P nuclear magnetic resonance (NMR) spectroscopy. The structure of complex 6 was determined by single-crystal X-ray diffraction (XRD) analysis. The phosphane ligands include the following: L1=bis(2-cyanoethyl)phenylphosphane, L2=(1R,2R)-2-(diphenylphosphano)-1-aminocyclohexane, L3=(1R,2R)-2-(diphenylphosphano)-1,2-diphenylethylamine, L4=(R)-2-(diphenylphosphano)-1-phenylethylamine, L5=(R)-1-(diphenylphosphano)-2-amino-3,3-dimethylbutane, L6=tricyclohexylphosphane, and L7=3-(diphenylphosphano)propylamine). The chemical structures of ligands (L1-L7) and their gold(I) complexes (1-7) are shown in FIGS. 2A-2G.

To improve gold(I) compound pharmacokinetics transport through the cell, tumor selectivity, lipophilicity, and antitumoral activity against cisplatin-resistant cancer cells, chiral phosphine ligands (L2-L5) or tricyclohexylphosphine (L6) were incorporated into metal-based drug candidates.

The complexes 1-7 were prepared by the reaction of [Au(IPr)Cl] with AgPF₆, followed by the addition of the respective phosphane ligand to the reaction mixture. The elemental analysis data corresponds to the proposed composition of [Au(IPr)(phosphane)] PF₆.

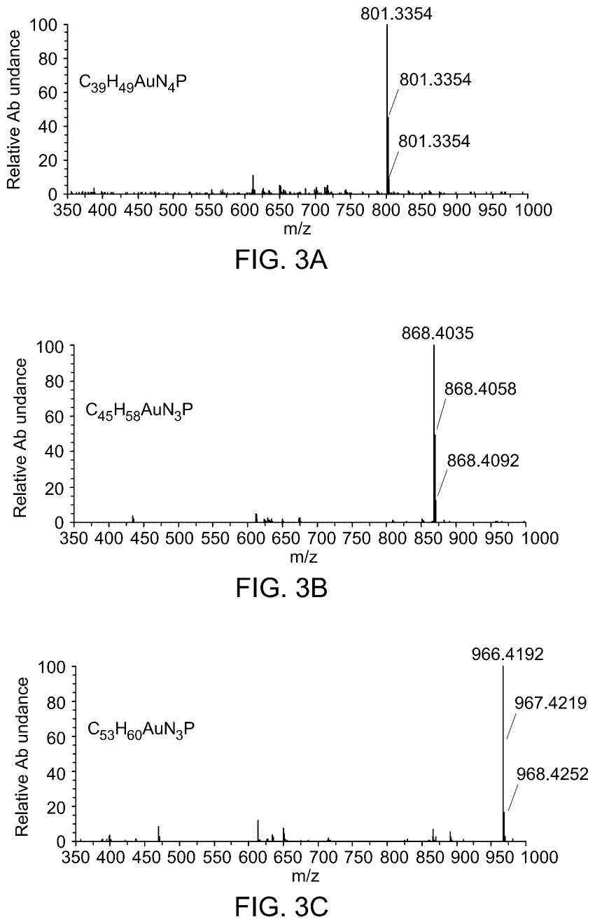

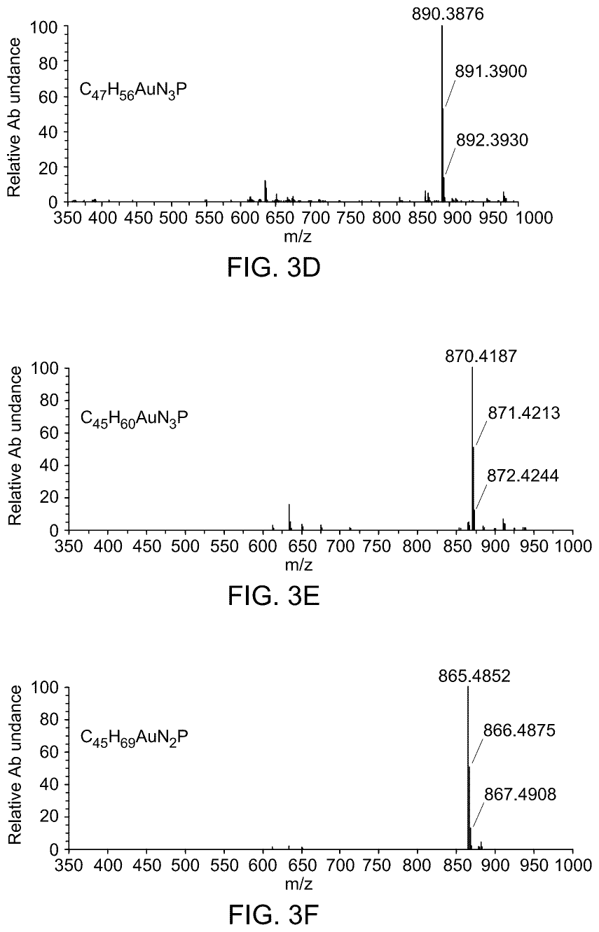

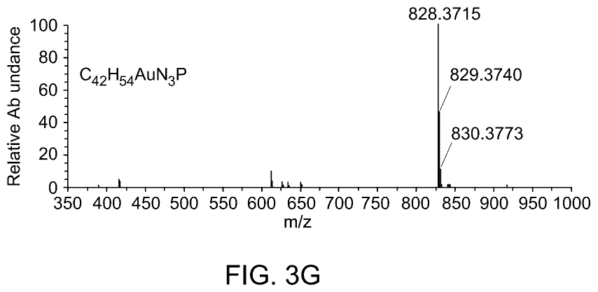

The X-ray data of the cyclic dinuclear gold(I) complex [Au₂(DCyPA)₂](PF₆)₂ based on bis[2-(dicyclohexylphosphano)ethyl]amine (DCyPA) showed no aurophilic interaction and no binding of Au(I) ions to the N-H moiety. Furthermore, the ¹H and ³¹P NMR data displayed the complex's stability under testing conditions. The high-resolution mass spectra analysis of the seven gold(I) purified complexes indicated that, in aqueous solutions, they were present preferentially as monocharged complexes, even though gold is bound to the ligands L2-L5 and L7, which contain potentially protonated amino groups. The m/z value of the monoisotopic form and the isotope pattern confirmed elemental compositions for the complexes (FIGS. 3A-3G). The X-ray crystallography analysis of complex 6 support the [Au(IPr)(phosphane)] PF₆ stoichiometry. The IR characteristic wavenumbers peaks for free ligands (L1-L7) and their complexes (1-7) are given in Tables 3.

TABLE 3
Mid FTIR wavenumbers (cm⁻¹) of free ligands (L1-L7) and gold(I) complexes (1-7)

| Compound | ν(N—H) | ν(═C—H) | ν(—C—H) | ν(C—H) | ν(C—P) | ν(C—N) | ν(C═C) | ν(C≡C) |
|---|---|---|---|---|---|---|---|---|
| Precursor | — | 3037 | 2960 | 1366 | 1109 | 1258 | 1462 | — |
| L1 | — | 3073 | 2933, 2911 | 1327 | 1095 | — | 1426 | 2250 |
| 1 | — | 3120 | 2956, 2874 | 1327 | 1108 | 1256 | 1467 | 2242 |
| L2 | 3334, 3259 | 3074 | 2916, 2844 | 1327 | 1099 | 1230 | 1478 | — |
| 2 | 3433, 3358 | 3170 | 2963, 2867 | 1328 | 1103 | 1215 | 1466 | — |
| L3 | 3371, 3302 | 3063 | 2903, 2854 | 1314 | 1071 | 1238 | 1484 | — |
| 3 | 3288, 3275 | 3171 | 2960, 2869 | 1329 | 1118 | 1214 | 1469 | — |
| L4 | 3376, 3350 | 3055 | 2939, 2838 | 1399 | 1066 | 1271 | 1485 | — |
| 4 | 3400, 3392 | 3168 | 2962, 2869 | 1329 | 1104 | 1214 | 1467 | — |
| L5 | 3381, 3301 | 3055 | 2955, 2867 | 1363 | 1084 | 1184 | 1479 | — |
| 5 | 3292, 3285 | 3172 | 2958, 2870 | 1329 | 1105 | 1217 | 1472 | — |
| L6 | — | — | 2932, 2849 | — | — | — | 1444 | — |
| 6 | — | 3167 | 2927, 2853 | 1328 | 1119 | 1272 | 1452 | — |
| L7 | 3367, 3292 | 3052 | 2929, 2854 | 1306 | 1095 | 1434 | 1586 | — |
| 7 | 3392, 3326 | 3148 | 2962, 2870 | 1329 | 1103 | 1215 | 1497 | — |

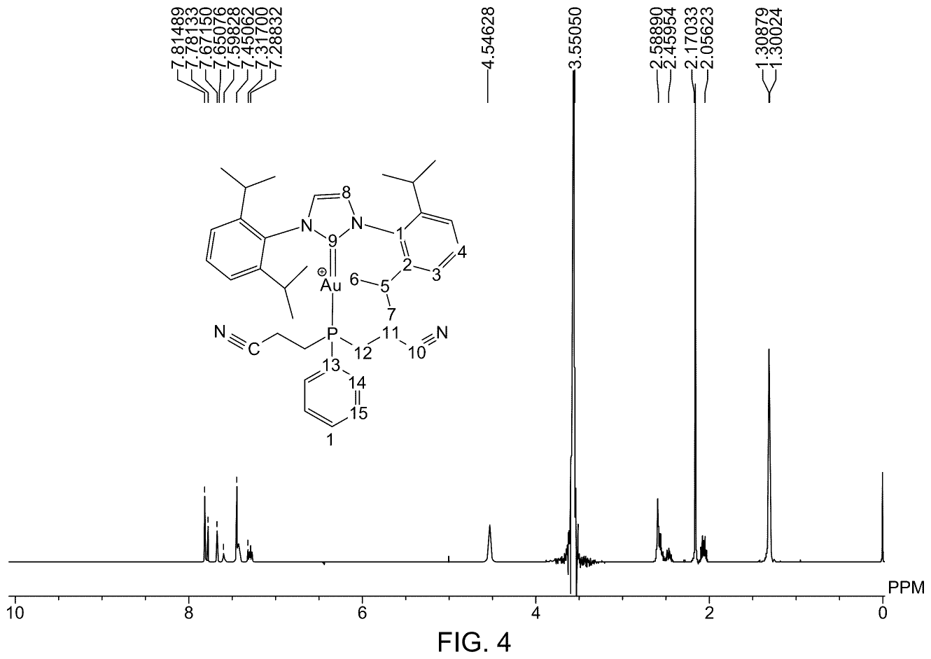

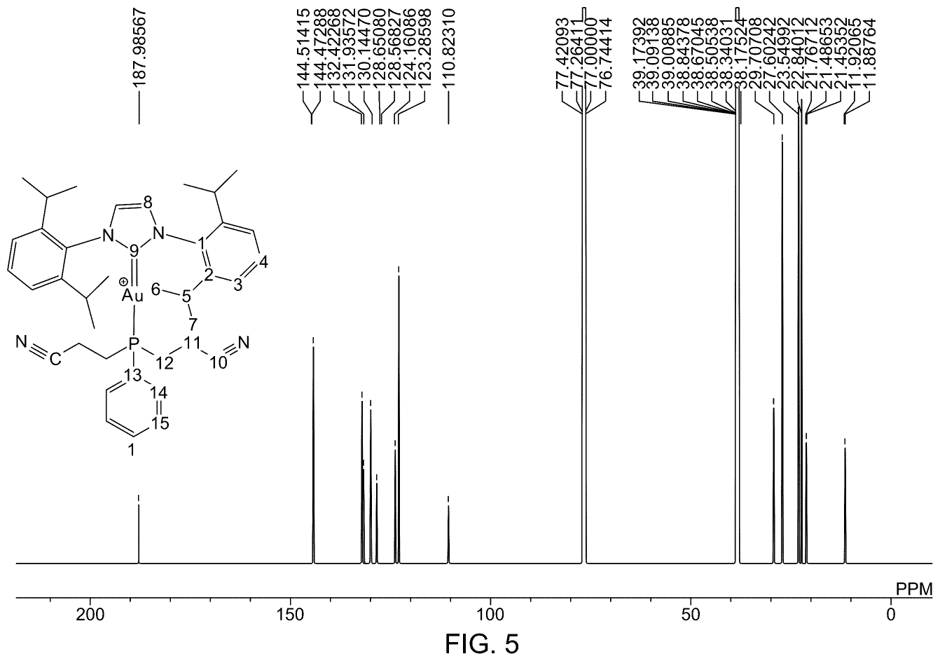

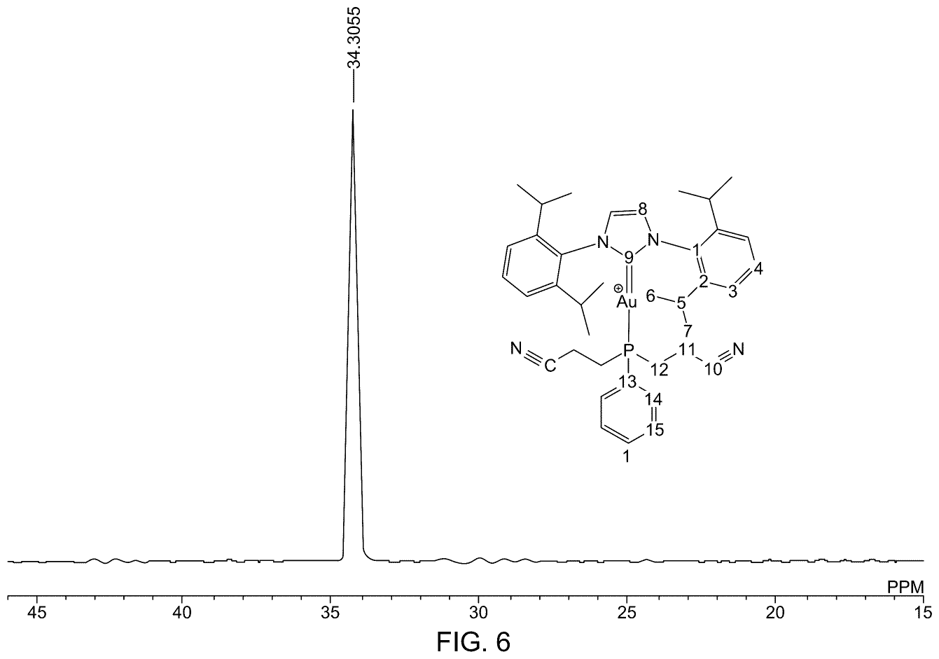

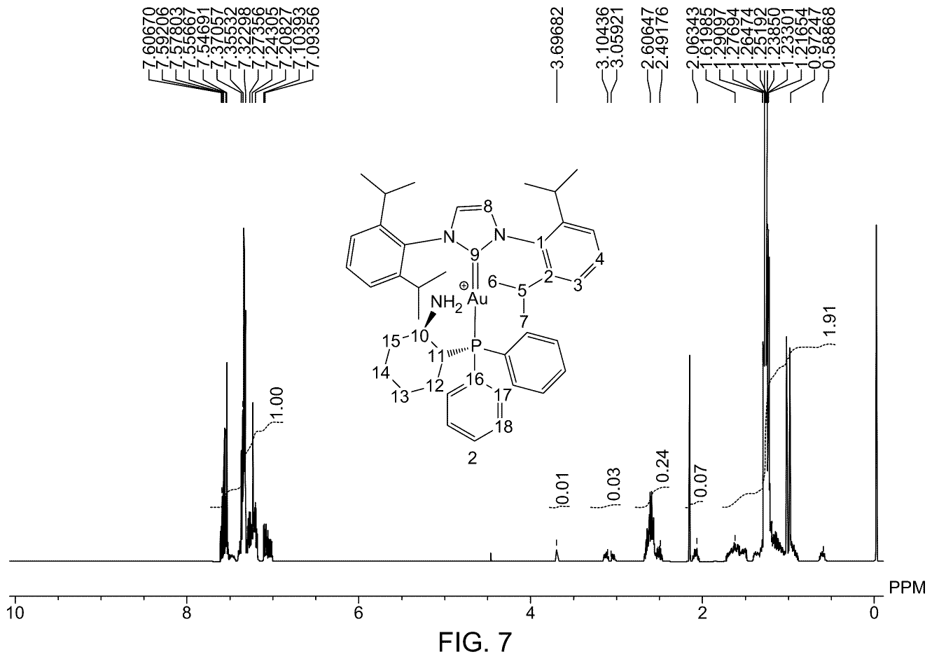

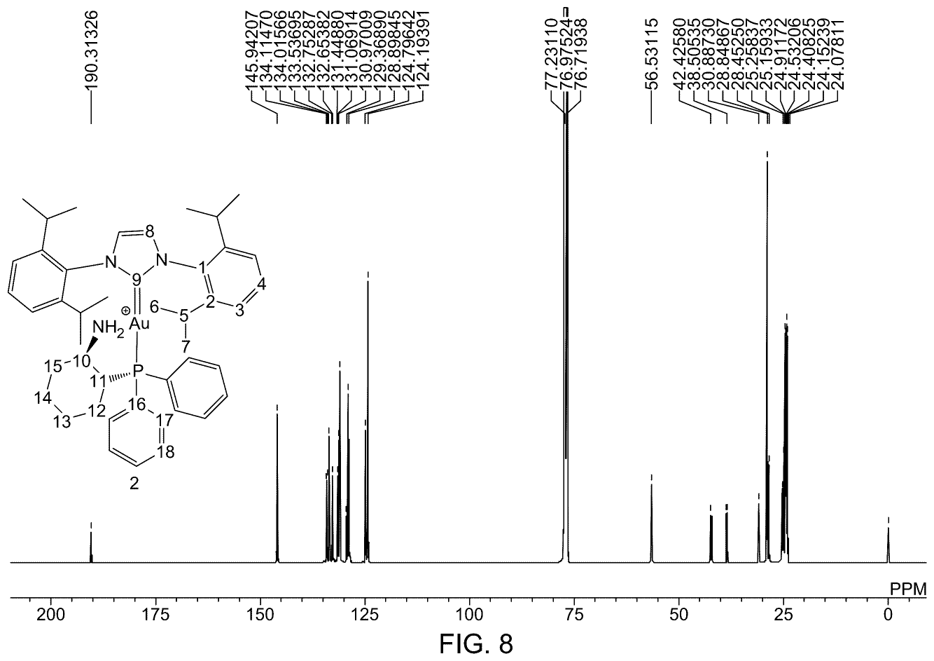

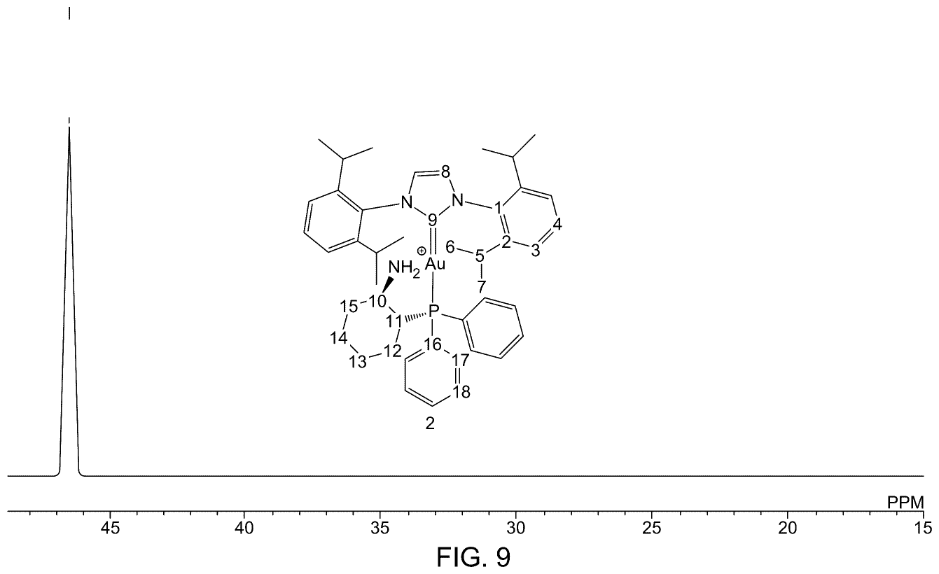

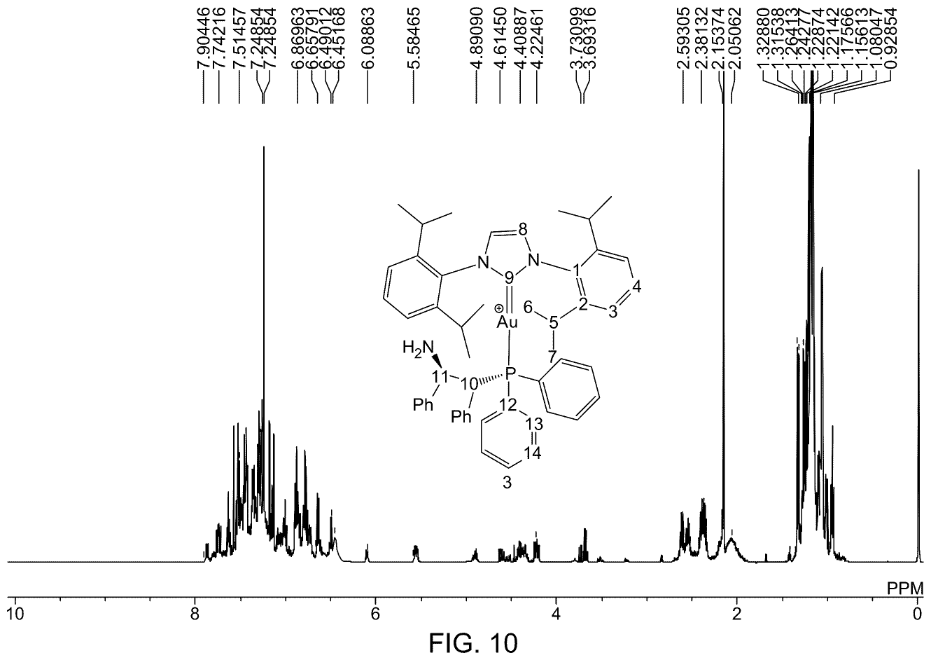

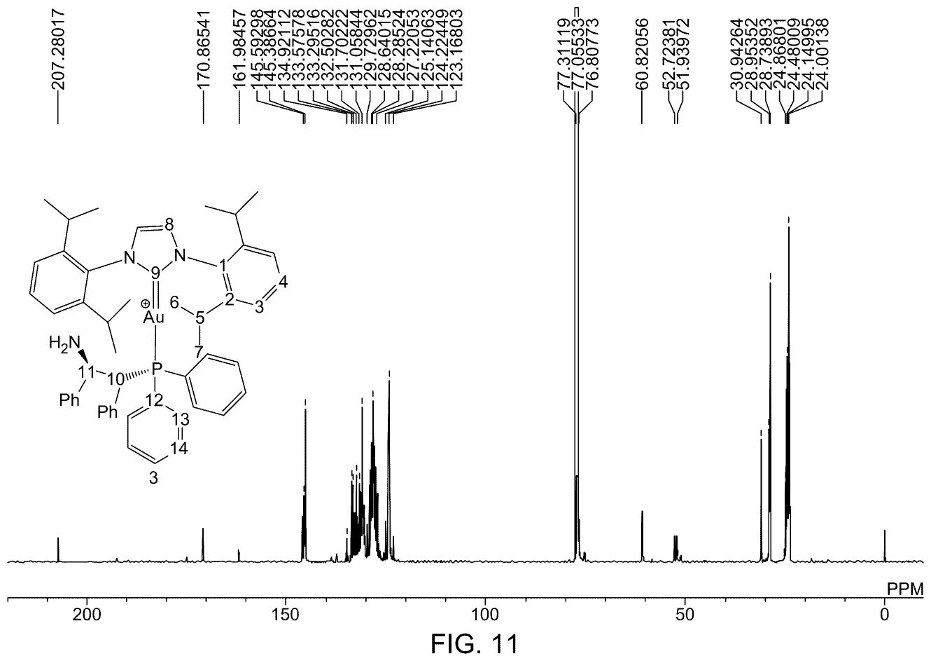

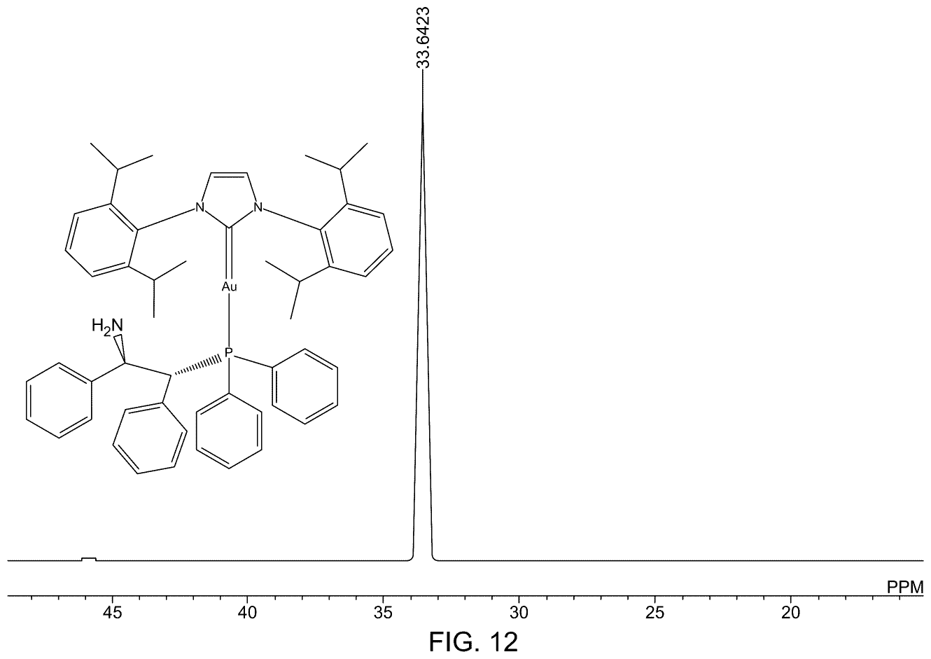

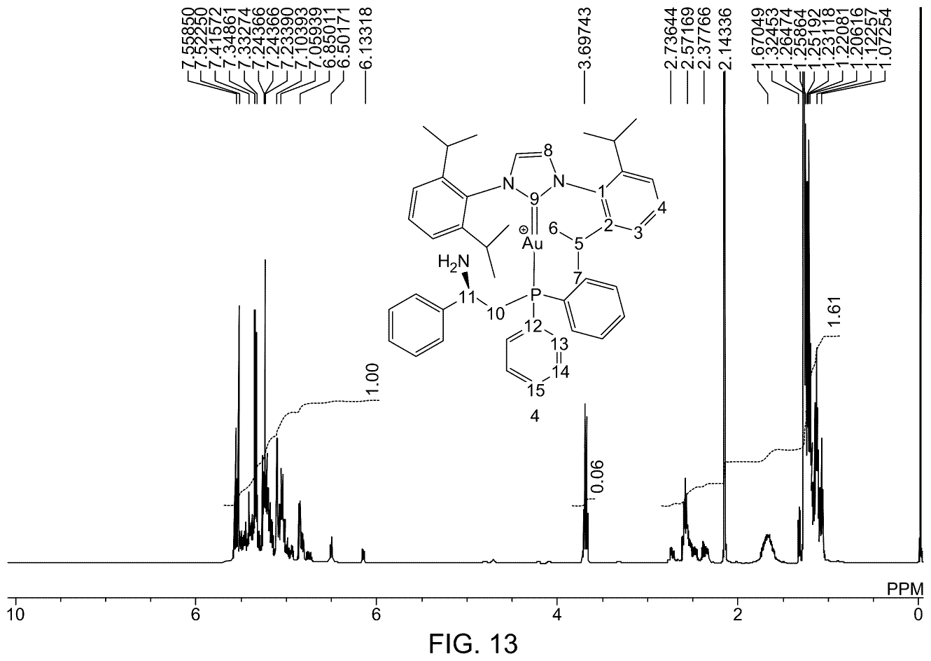

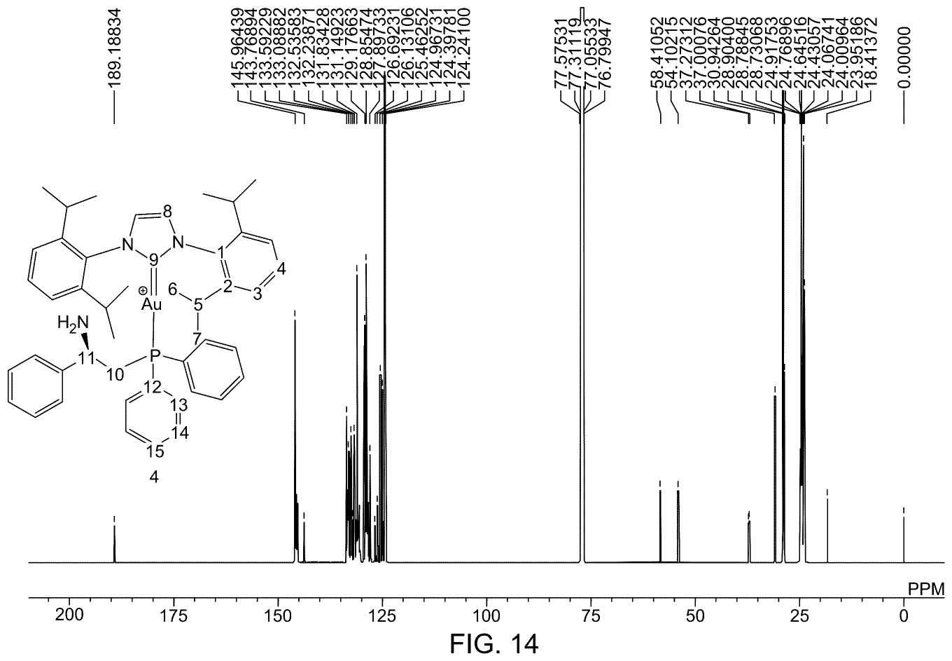

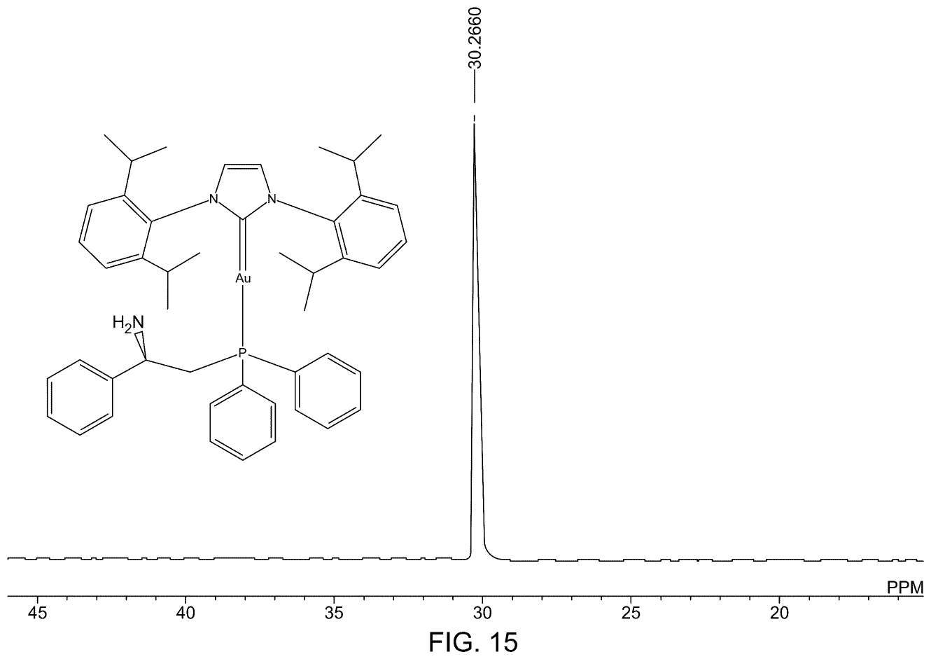

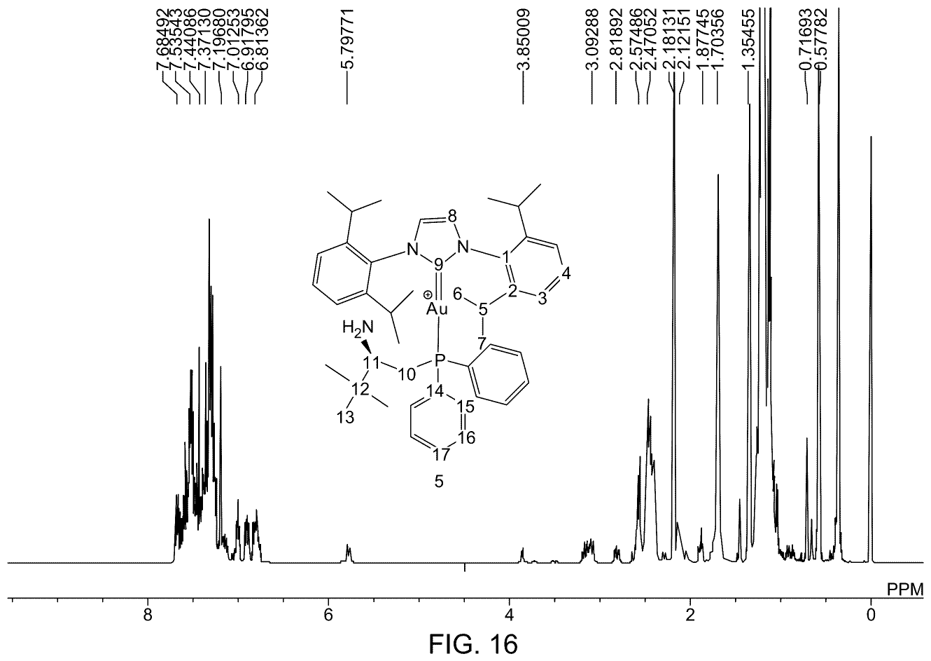

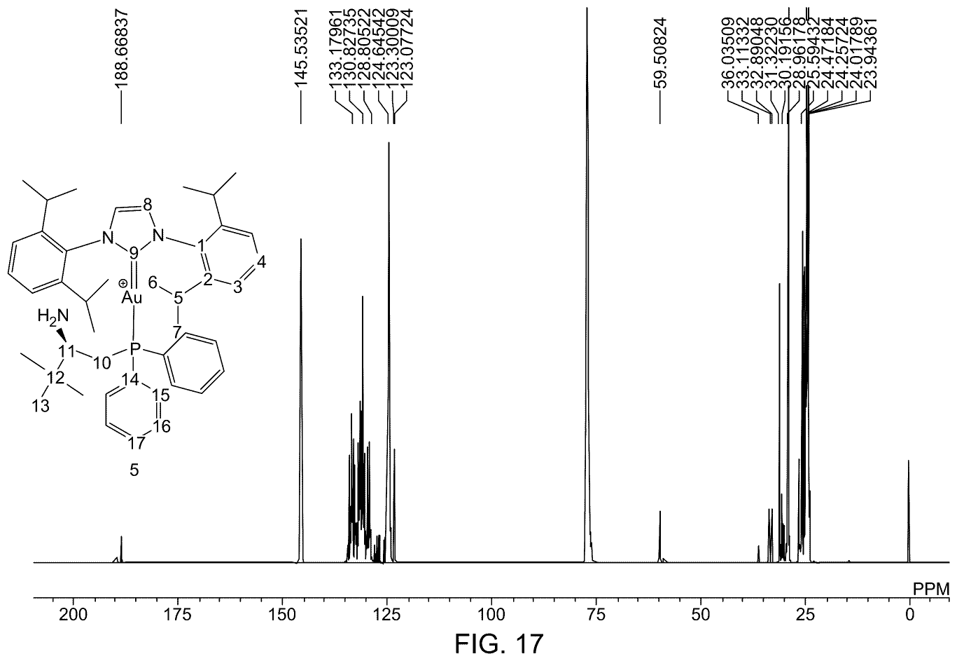

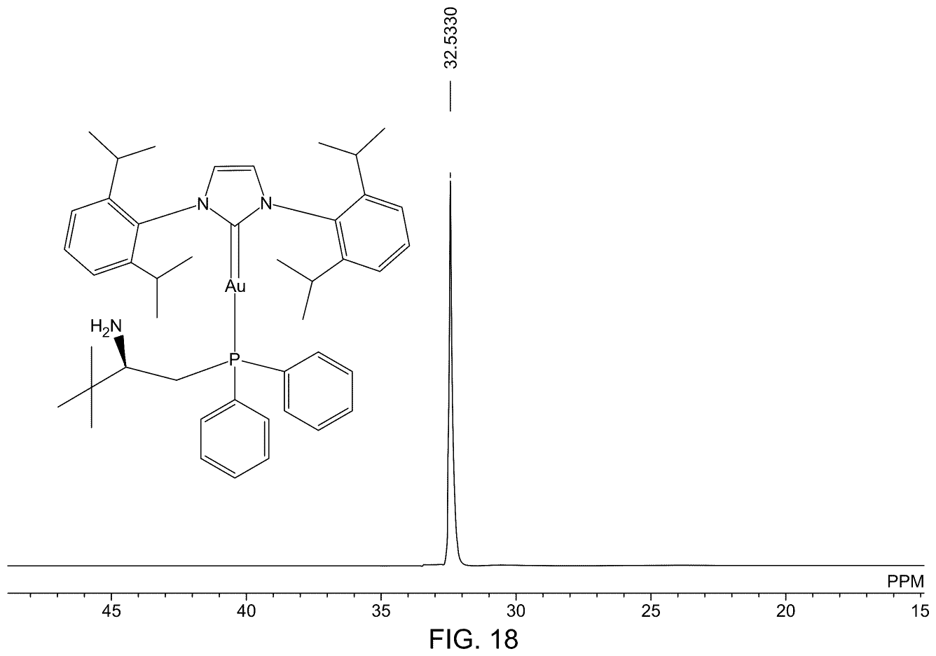

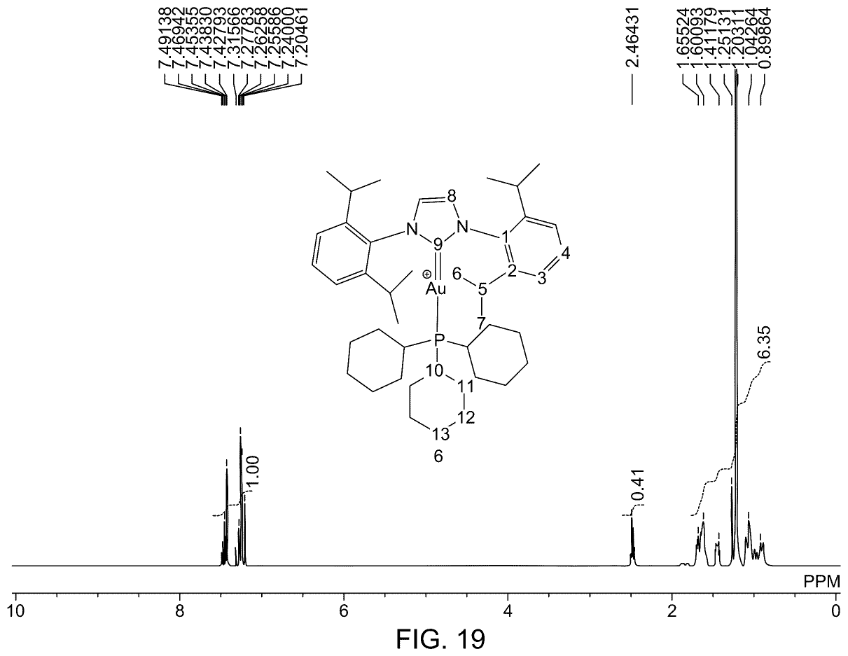

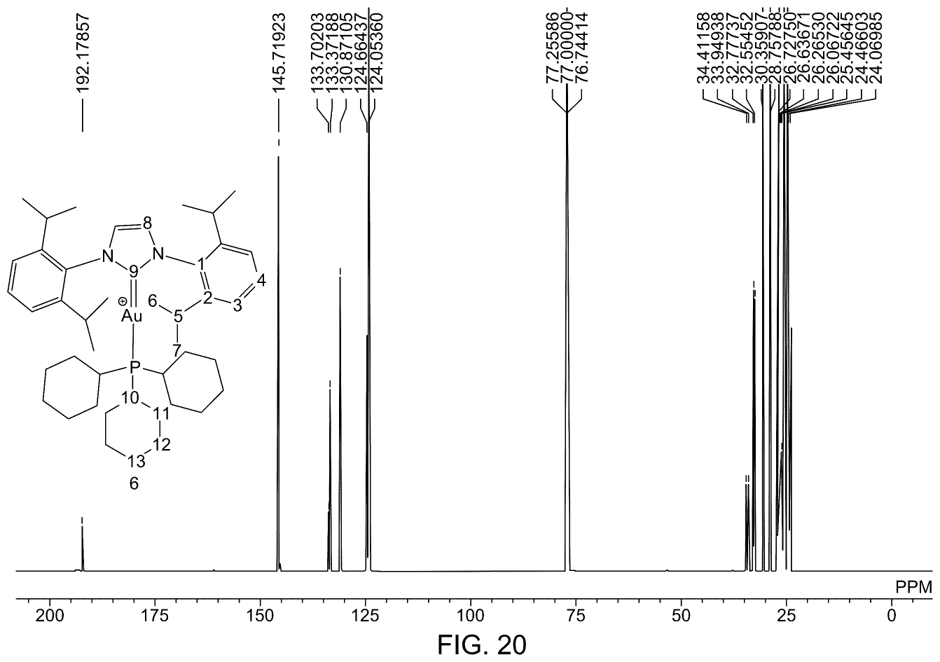

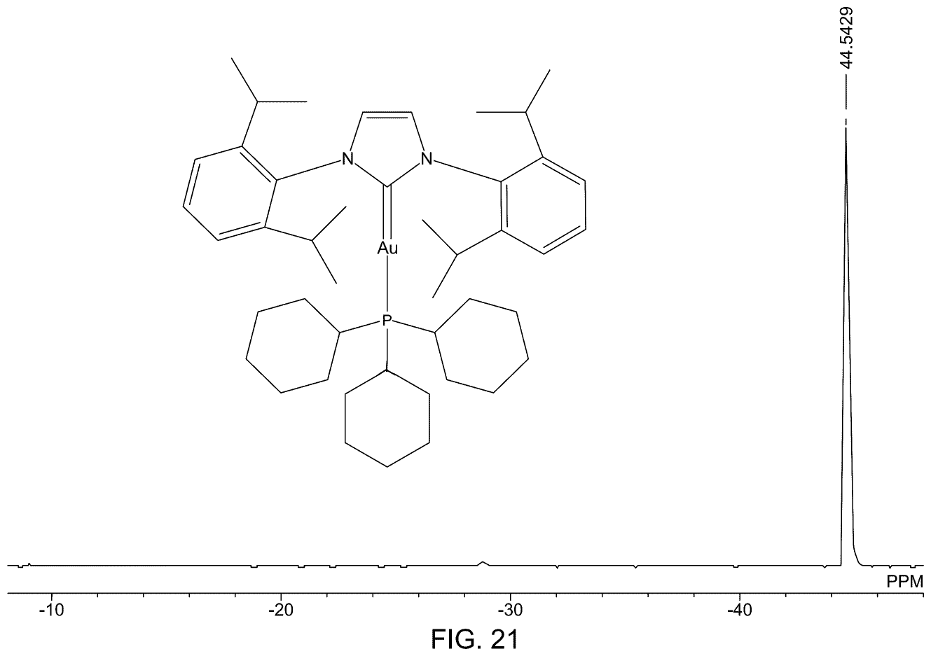

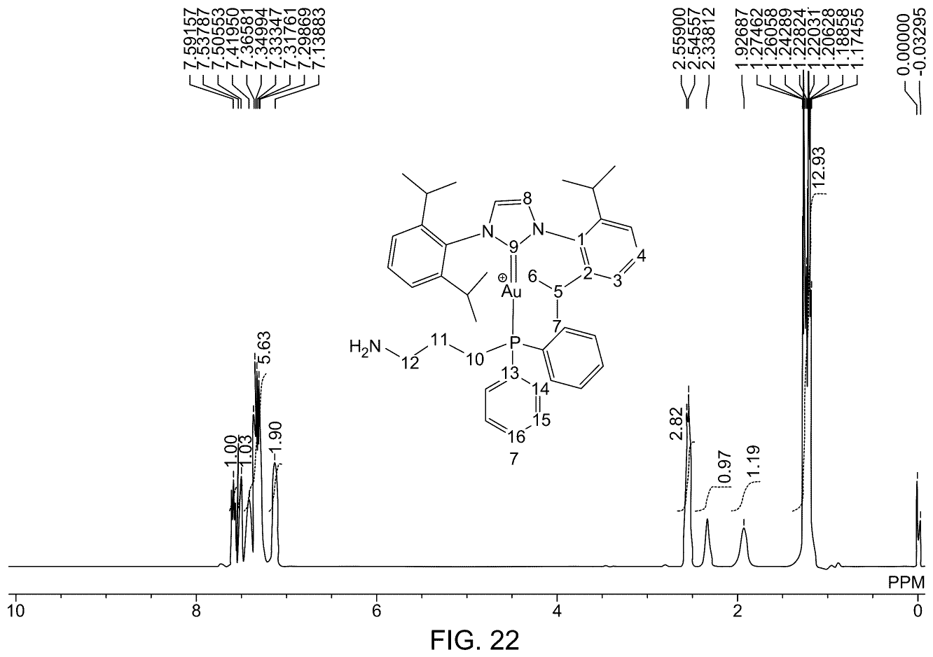

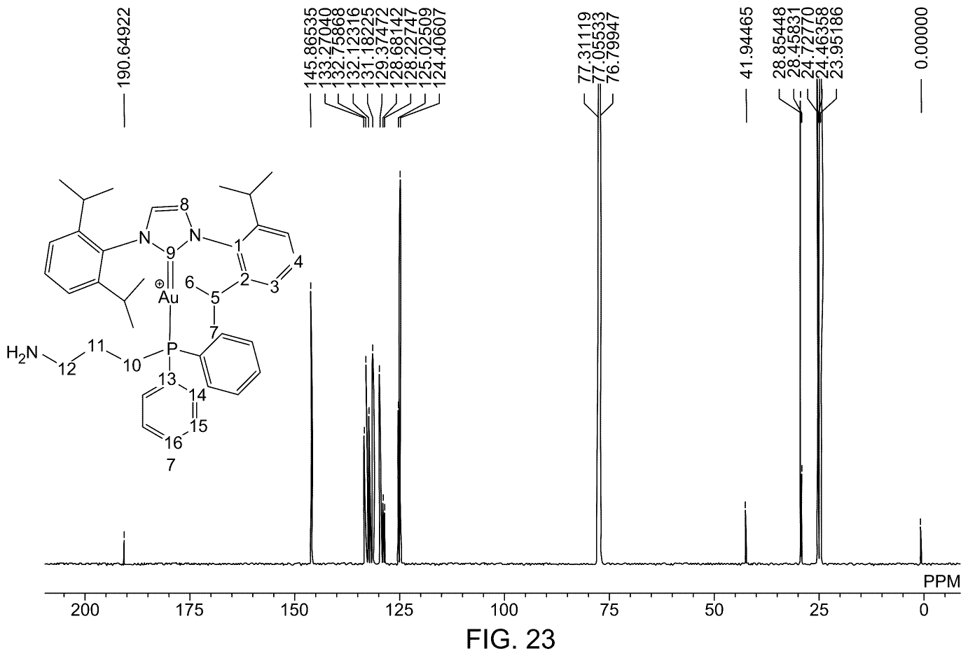

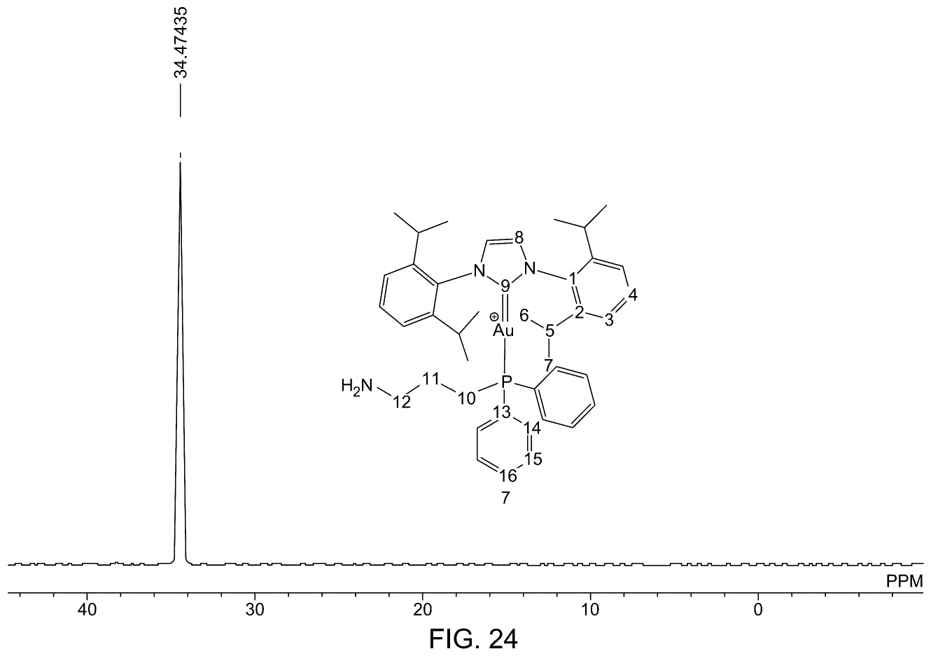

The FTIR spectra showed weak absorbance in the region of 3049-3078 cm⁻¹ assigned to the benzene ring═C—H stretching for the free ligands and precursor. This band was shifted to a higher frequency (in the range 3120-3175 cm⁻) in each complex. The v(C═N) band for free L1 appears at 2250 cm⁻, while in complex 1, it was shifted towards a lower wavenumber (2242 cm⁻), indicating the complexation of L1 to gold(I). The bands around 1100 cm⁻¹ correspond to the P-C stretches of phosphanes. ¹H, ¹³C, and ³¹P NMR spectroscopy analysis results for complexes 1-7 are depicted in FIGS. 4-24, respectively. The ¹H NMR chemical shifts of free ligands and their complexes (1-7) are given in Table 4.

TABLE 4
¹H NMR chemical shifts (ppm) for free ligands and gold(I)
complexes (1-7) in CDCl₃ (Values in parentheses represent corresponding
(1J or 2J) ¹³C—³¹P coupling constants)

| Ligand/Complex | 3H | 4H | 5H | 6H | 7H | 8H | 10H |
|---|---|---|---|---|---|---|---|
| Precursor | 7.39 d | 7.55 t | 2.46 m | 1.33 d | 1.21 d | 7.98 s | — |
| L1 | — | — | — | — | — | — | — |
| 1 | 7.31 d | 7.67 t | 2.58 m | 1.31 d | 1.30 d | 7.28 s | — |
| L2 | — | — | — | — | — | — | 2.59 m |
| 2 | 7.35 d | 7.55 t | 2.60 m | 1.26 d | 1.23 d | 7.10 s | 3.08 m |
| L3 | — | — | — | — | — | — | 4.41 d |
| 3 | 6.65 d | 7.51 t | 2.59 m | 1.31 d | 1.22 d | 6.08 s | 4.61 d (4.6) |
| L4 | — | — | — | — | — | — | 2.73, 183 d |
| 4 | 6.86 d | 7.52 t | 2.57 m | 1.26 d | 1.23 d | 6.50 s | 2.37 m (5.8) |
| L5 | — | — | — | — | — | — | 2.90 dd |
| 5 | 6.81 d | 6.91 t | 2.87 m | 1.35 d | 1.27 d | 5.79 s | 3.09 s |
| L6 | — | — | — | — | — | — | 1.66 m |
| 6 | 7.42 d | 7.49 t | 2.46 m | 1.25 d | 1.20 d | 7.20 s | 1.41 m (12.2) |
| L7 | — | — | — | — | — | — | 1.50 m |
| 7 | 7.50 d | 7.59 t | 2.33 m | 1.26 d | 1.20 d | 7.13 s | 1.27 m (23.2) |

| Ligand/Complex | 11H | 12H | 13H | 14H | 15H | NH |
|---|---|---|---|---|---|---|
| Precursor | — | — | — | — | — | — |
| L1 | 2.36 t | 2.10 t | — | 7.53 dd | 7.45 dd | — |
| 1 | 2.45 t | 2.05 t (8.5) | — | 7.59 dd | 7.45 dd | — |
| L2 | 1.69 m | 2.06, 1.21 m | 2.06 m | 0.87 m | — | 2.02 |
| 2 | 1.61 m (17.1) | 2.49, 1.25 m | 2.06 m | 0.97 m | — | 2.12 |
| L3 | 4.40 dd | 7.82 m | 7.14 m | 7.44 m | — | 1.8 |
| 3 | 4.40 dd | 7.92 m | 6.86 m | 7.74 m | — | 2.15 |
| L4 | 3.99 d | 7.41 m | 7.11 m | — | — | 1.99 |
| 4 | 3.69 d | 7.55 m | 7.10 m | — | — | 1.67 |
| L5 | 2.40, 1.83 d | 7.78 m | 7.34 m | 0.94 m | — | 2.14 |
| 5 | 2.47 m (13.4) | 7.68 m | 7.53 m | 0.71 m | — | 2.18 |
| L6 | 1.89, 1.22 d | 1.80, 138 d | 1.73, 1.58 | — | — | — |
| 6 | 1.76, 0.89 | 1.65, 1.04 | 1.60, 1.18 | — | — | — |
| L7 | 1.99 m | 2.69 t | 7.25 m | 7.35 t | — | 1.09 |
| 7 | 1.92 m | 2.54 t | 7.33 m | 7.41 t | — | 1.17 |

¹H NMVR data showed shifts in a carbene moiety between the precursor and the prepared complexes (1-7). ¹H NMR chemical shift of NH in free ligands (L2-L5 and L7) was observed in a range of 1.09-2.02 ppm and shifted downfield for their complexes (2-5 and 7) in a range of 1.17-2.21 ppm. For each complex, the ¹H NMR data of H—C—P moiety were observed downfield with respect to the free ligands. The ¹³C NMR chemical shifts for free ligands and their complexes are summarized in Table 5A.

TABLE 5
¹³C NMR chemical shifts (ppm) for free ligands and gold(I) complexes (1-7) in
CDCl₃ (values in parentheses represent corresponding (1J or 2J) ¹³C—³¹P coupling constants)

| Ligand/complex | C1 | C2 | C3 | C4 | C5 | C6 | C7 | C8 | Au═C |
|---|---|---|---|---|---|---|---|---|---|
| Precursor | 145.5 | 133.9 | 130.7 | 123.0 | 28.8 | 24.5 | 24.0 | 124.2 | 175.3 |
| L1 | | | | | | | | | |
| 1 | 144.5 | 132.4 | 131.1 | 123.3 | 27.6 | 23.5 | 22.8 | 124.2 | 187.9 |
| L2 | | | | | | | | | |
| 2 | 145.9 | 133.5 | 131.0 | 124.2 | 28.4 | 24.9 | 24.4 | 124.8 | 190.3 |
| L3 | | | | | | | | | |
| 3 | 145.4 | 133.5 | 131.0 | 123.2 | 28.7 | 24.4 | 24.1 | 124.2 | 207.2 |
| L4 | | | | | | | | | |
| 4 | 146.0 | 133.6 | 131.1 | 124.4 | 28.7 | 24.7 | 24.0 | 124.2 | 189.1 |
| L5 | | | | | | | | | |
| 5 | 145.5 | 133.2 | 130.8 | 123.3 | 29.0 | 24.7 | 24.0 | 124.6 | 188.6 |
| L6 | | | | | | | | | |
| 6 | 0.7 | 133.4 | 130.9 | 124.0 | 28.8 | 24.4 | 24.0 | 124.7 | 192.1 |
| L7 | | | | | | | | | |
| 7 | 145.8 | 133.3 | 131.2 | 124.2 | 28.8 | 24.7 | 23.9 | 125.0 | 190.6 |

| Ligand/Complex | C10 | C11 | C12 | C13 | C14 | C15 | C16 | C17 | C18 |
|---|---|---|---|---|---|---|---|---|---|
| Precursor | — | — | — | — | — | — | — | — | — |
| L1 | 119.1 | 14.1 | 23.9 | 133.2 | 132.6 | 129.2 | — | — | — |
| 1 | 110.8 | 11.9 (6.7) | 21.4 (6.6) | 128.6 (16.7) | 131.9 | 128.6 | — | — | — |
| L2 | 52.5 | 43.8 | 36.6 | 26.2 | 24.8 | 27.3 | 135.6 | 132.3 | 128.3 |
| 2 | 56.5 | 42.4 | 38.8 | 25.2 | 24.4 | 28.8 | 134.1 (20) | 132.7 (20) | 129.4 |
| L3 | 58.8 | 53.4 | 143.6 | 137.6 | 136.9 | 133.7 | 128.7 | 127.6 | 126.2 |
| 3 | 60.8 | 52.7 (158) | 145.6 | 134.9 | 133.5 | 133.2 | 128.6 | 127.2 | 125.1 |
| L4 | 53.7 | 39.4 | 145.6 | 138.4 | 132.9 | 128.7 | 126.2 | — | — |
| 4 | 58.4, 54.1 | 37.0 (55) | 143.7 | 133.6 | 132.2 | 127.9 | 126.1 | — | — |
| L5 | 32.3 | 57.4 | 34.6 | 25.7 | 139.6 | 133.4 | 128.8 | — | — |
| 5 | 59.5 | 32.9 (45) | 31.3 | 25.6 | 133.2 | 130.7 | 128.8 | — | — |
| L6 | 35.5 | 31.0 | 27.6 | 26.5 | — | — | — | — | — |
| 6 | 34.4 (93) | 32.8 (45) | 26.7 | 25.5 | — | — | — | — | — |
| L7 | 43.3 | 30.0 | 25.2 | 138.6 | 132.4 | 128.3 | — | — | — |
| 7 | 41.9 | 28.5 | 24.5 | 132.8 | 129.4 | 128.8 | — | — | — |

In ¹³C NMVR, the C═Au chemical shift of precursor was observed at 175.3 ppm, whereas for the prepared complexes, it was observed at a downfield position in the range 187.9-207.2 ppm, supporting the formation of the complexes. ³¹P NMR chemical shifts (ppm) for free ligands and gold(I) complexes (1-7) are given in Table 6.

TABLE 6
³¹P NMR chemical shifts (ppm) for free ligands and
gold(I) complexes (1-7) in CDCl₃.

| Compound | ³¹P δ(ppm) |
|---|---|
| Precursor | — |
| L1 | −25.14 |
| 1 | 34.30 |
| L2 | −9.22 |
| 2 | 46.46 |
| L3 | −10.65 |
| 3 | 33.64 |
| L4 | −23.59 |
| 4 | 30.26 |
| L5 | −21.24 |
| 5 | 32.53 |
| L6 | 9.33 |
| 6 | −44.54 |
| L7 | −17.80 |
| 7 | 34.47 |

The resonance values for free ligands were in the range of −25.14 to −9.22 ppm, whereas those of their complexes were observed in the downfield region (−44.54 to 46.46 ppm) due to back-donation from gold ion to π* orbital of P-C bond. For L6 and complex 6, the signal appeared at 9.33 ppm and −44.54 ppm, respectively.

### X-Ray Structure Determination of Complex 6

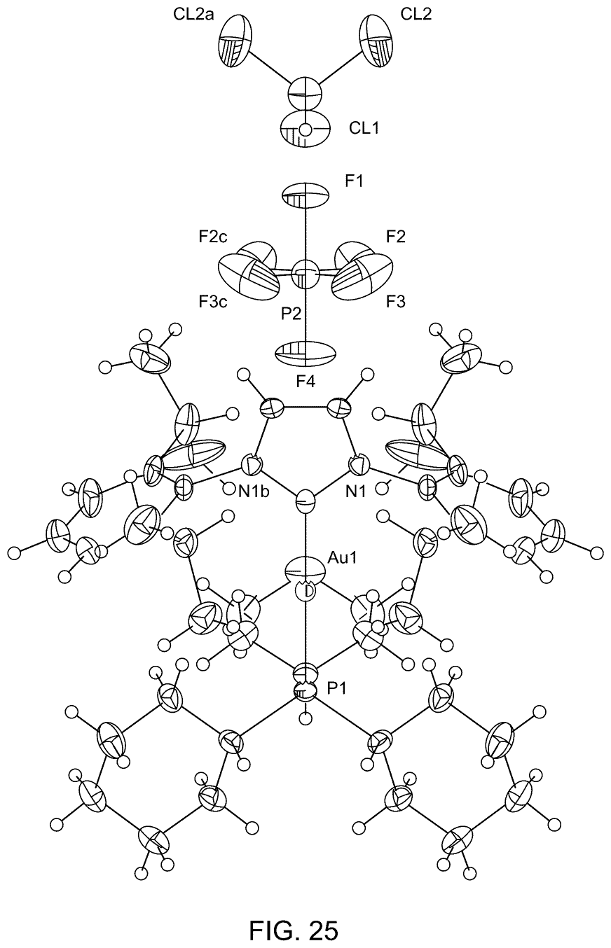

The molecular structure of complex 6 is illustrated in FIG. 25. The complex crystallized with a PF₆⁻ anion and a chloroform molecule as monosolvate. The cation, anion, and solvent molecule were located on a mirror plane that bisects each component. The selected bond distances and bond angles for complex 6 are given in Table 7.

TABLE 7
Selected bond lengths and bond angles for complex 6

| Bond length (Å) | | Bond angle (deg) | |
|---|---|---|---|
| P1—Au1 | 2.295 (3) | C1—Aul—P1 | 179.9 (3) |
| C1—Au1 | 2.022 (11) | C15—PI—Aul | 110.7 (3) |
| NI—C1 | 1.355 (8) | C21—PI—Aul | 110.1 (3) |
| P1—C15 | 1.386 (7) | N1—Au1—Cl | 128.2 (4) |
| PI—C21 | 1.387 (9) | | |

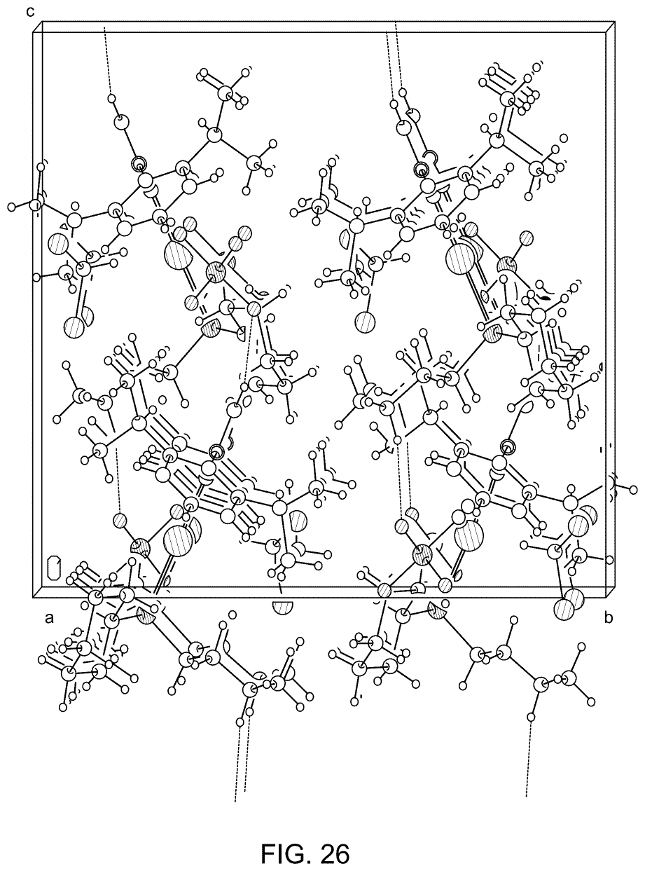

The coordination geometry around gold(I) ion was ideally linear with an angle (C—Au—P) of 179.9 (3°). The Au—P and Au—C bond lengths (A) were 2.295(3) and 2.022(11), respectively, which agree with literature. The structure of complex 6 is very similar to the other phosphane-gold(I)-carbene complexes. Within the crystal packing (FIG. 26), the cations and anions were linked via C—H . . . F hydrogen bonds forming chains along the c-axis direction. The non-traditional hydrogen bond for complex 6, PF₆ . . . H—CCl₃ was found between F₁ of counterion (PF₆⁻) and the hydrogen atom of the CHCl₃ solvent. The hydrogen bond length and bond angle were H . . . F1 2.25(5) (A) and C—H F1 170°, respectively.

### In Vitro Cytotoxicity of Gold(I) Complexes in Ovarian Cancer Models

To assess the growth inhibition potential of the gold(I) complexes 1-7, OvCa cell line, A2780, and its cisplatin-resistant clone, A2780cis, were used. Both cisplatin and auranofin were included for comparative purposes. The half-maximal inhibitory concentration (IC₅₀) of cisplatin was 1.5 μM for A2780 and 10.4 μM for A2780cis (Table 8).

TABLE 8
Growth inhibition by complexes 1-7 in ovarian cancer cell lines,
A2780, and cisplatin-resistant A2780cis

| Compound | A2780 | A2780cis | Fold resistance (A2780cis/A2780) |
|---|---|---|---|
| Cisplatin | 1.50 ± 0.11 | 10.40 ± 0.98 | 6.93 |
| Auranofin | 0.51 ± 0.07 | 1.91 ± 0.22 | 3.7 |
| Complex 1 | 2.00 ± 0.19 | 3.55 ± 0.35 | 1.78 |
| Complex 2 | 0.50 ± 0.04 | 0.80 ± 0.04 | 1.6 |
| Complex 3 | 0.78 ± 0.06 | 1.14 ± 0.10 | 1.46 |
| Complex 4 | 0.82 ± 0.05 | 1.17 ± 0.15 | 1.43 |
| Complex 5 | 0.75 ± 0.07 | 0.92 ± 0.08 | 1.23 |
| Complex 6 | 0.14 ± 0.01 | 0.12 ± 0.01 | 0.86 |
| Complex 7 | 1.42 ± 0.12 | 1.80 ± 0.15 | 1.27 |

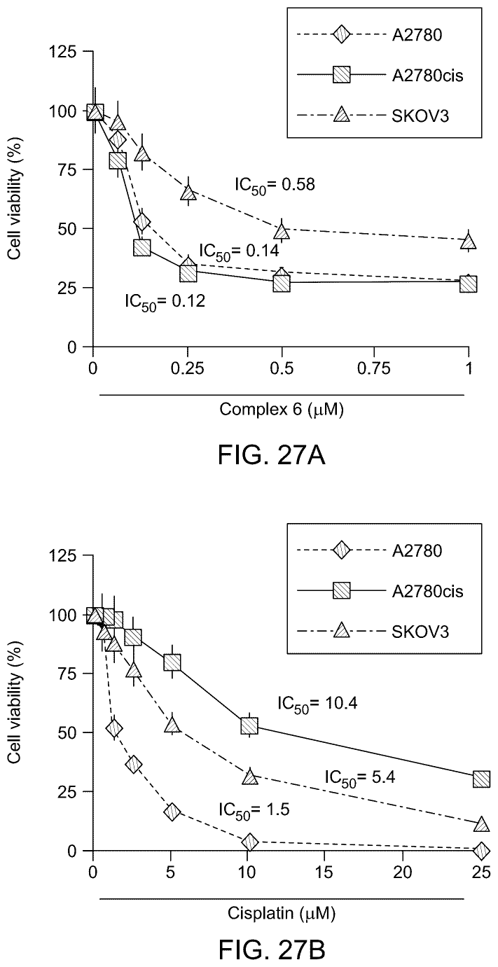

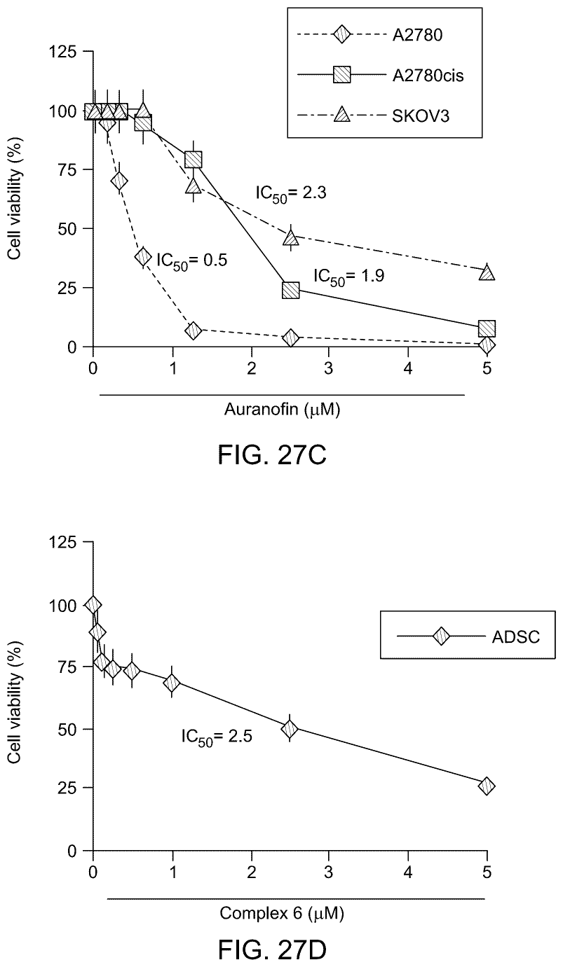

The IC₅₀ of auranofin was 0.5 μM for A2780 and 1.9 μM for A2780cis, as can be observed in Table 8. A2780cis was less sensitive to both cisplatin and auranofin, showing a fold resistance (FR) (IC₅₀ A2780cis/IC₅₀ A2780) of 6.93 and 3.7 for cisplatin and auranofin, respectively. In A2780 cells, complexes 2, 3, 4, 5, and 6 were more active than cisplatin, with an IC₅₀ ranging from 0.14 to 0.82 μM, while complex 1 and complex 7 had an IC₅₀ value similar to or higher than cisplatin. In A2780cis cells, the seven gold(I) complexes were more active than cisplatin, with IC₅₀ ranging from 0.12 to 3.55 μM. The fold resistance (FR) between the two cell lines for the tested complexes ranged from 0.86 to 1.78, thus excluding cross-resistance to cisplatin in these cell lines (Table 8). In A2780 cells, complex 6 was more active than auranofin, whereas in A2780cis, each complex, excluding complex 1, were more active than auranofin. Dose-response curves with complex 6, cisplatin, and auranofin in A2780, A2780cis, and SKOV3 cells are shown in FIGS. 27A-27C. The large cyclohexyl groups on the phosphane ligand (Cy₃P) may be responsible for the potency of complex 6. The complexes containing chiral phosphine ligands (L2-L5) displayed moderate activity, while the complexes containing ligands (L1 and L7) were the least active, as can be observed from Table 8. This observation is in line with other reports, which describe high cytotoxic effects of Cy₃P-containing gold(I) complexes with respect to (cyanoethyl)₃P complexes and aminophosphane complexes [Ang, K. P.; Chan, P. F.; Hamid, R. A. Antiproliferative Activity Exerted by Tricyclohexylphosphanegold(I) n-Mercaptobenzoate against MCF-7 and A2780 Cell Lines: The Role of P53 Signaling Pathways. Biometals 2021, 34 (1), 141-160; and Ang, K. P.; Chan, P. F.; Hamid, R. A. Induction of Apoptosis on Ovarian Adenocarcinoma Cells, A2780 by Tricyclohexylphosphanegold (I) Mercaptobenzoate Derivatives via Intrinsic and Extrinsic Pathways. J. Biol. Inorg. Chem. 2021, 26 (7), 833-853, incorporated herein by reference in its entirety]. The low overall cytotoxic activity of the complexes bearing ligands L2-L5 and L7 in comparison to the complexes bearing ligands without amino groups may be explained by the occupancy of a potential reactive site of the gold via the side ligand amino group.

Preclinical activity of complex 6 Complex 6 was found to be the most potent among the prepared gold(I) complexes, characterized by the lowest IC₅₀ in both cisplatin sensitive and resistant A2780 cells and FR<1 (Table 8). Therefore, it was chosen for further analysis. Preclinical studies were also performed with SKOV3 OvCa cells, capable of spontaneously forming 3D-(MCTSs). SKOV3 cells had IC₅₀=0.58 μM for complex 6, IC₅₀=5.4 μM for cisplatin, and IC₅₀=2.3 μM for auranofin (FIGS. 27A-27C).

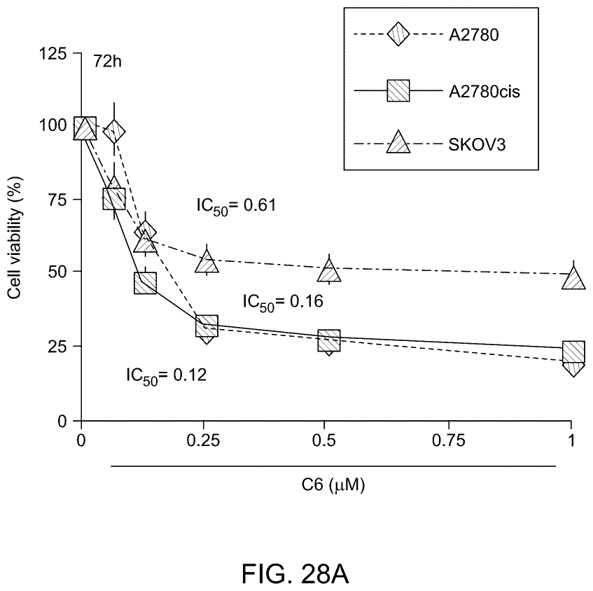

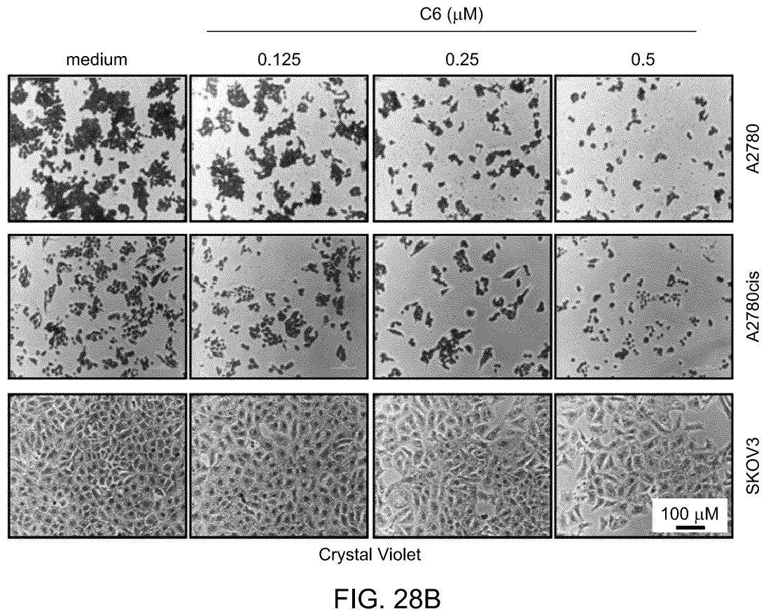

The growth inhibition of OvCa cells by complex 6 was further demonstrated using crystal violet staining (FIG. 28A). The representative images of OvCa cells stained with crystal violet after (72 hours) treatment with complex 6 are shown in FIG. 28B.

The complex 6 activity was also evaluated in normal human adipose-derived stem cells (ADSCs). It was found that complex 6 was less toxic in ADSCs (IC₅₀=2.5 μM) than in OvCa cells (IC₅₀ ranging from 0.12 to 0.58 μM) (FIG. 27D and FIG. 27A).

### Mechanism of Action of Complex 6 in A2780 and A2780cis Cells

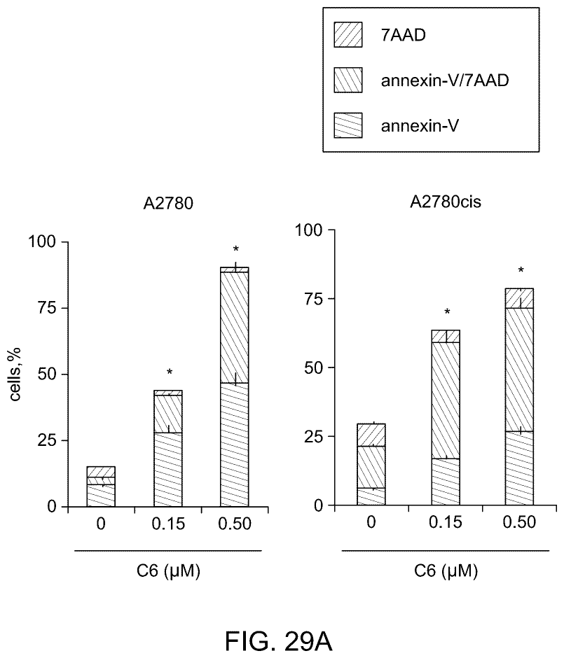

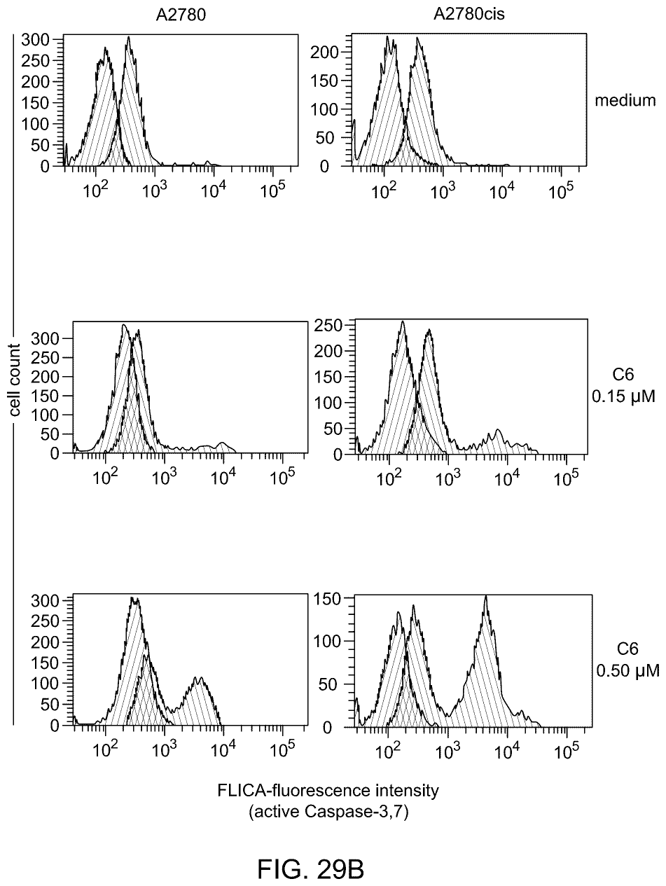

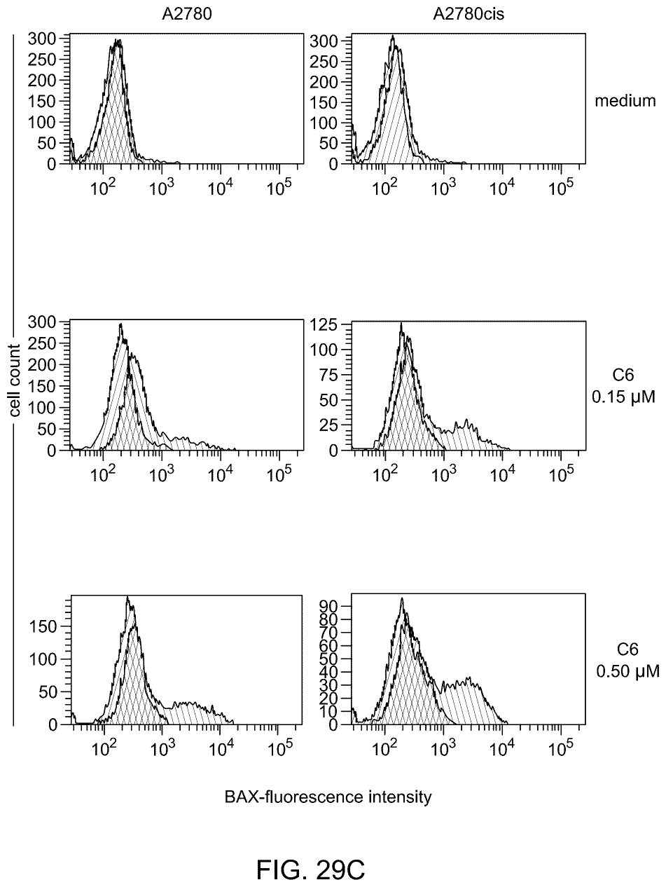

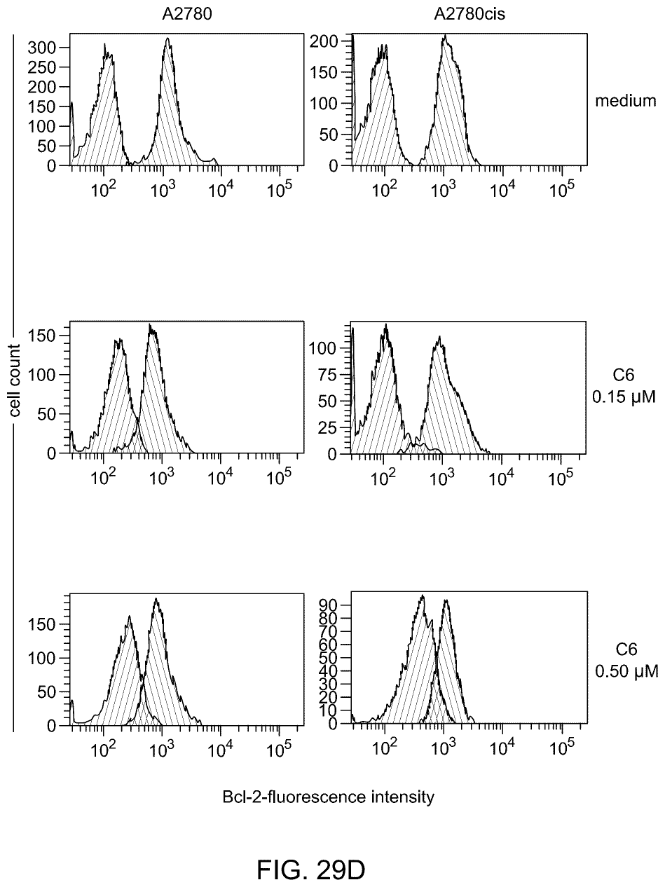

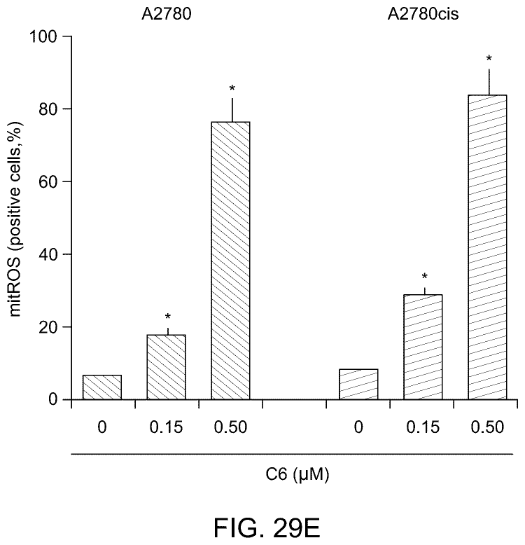

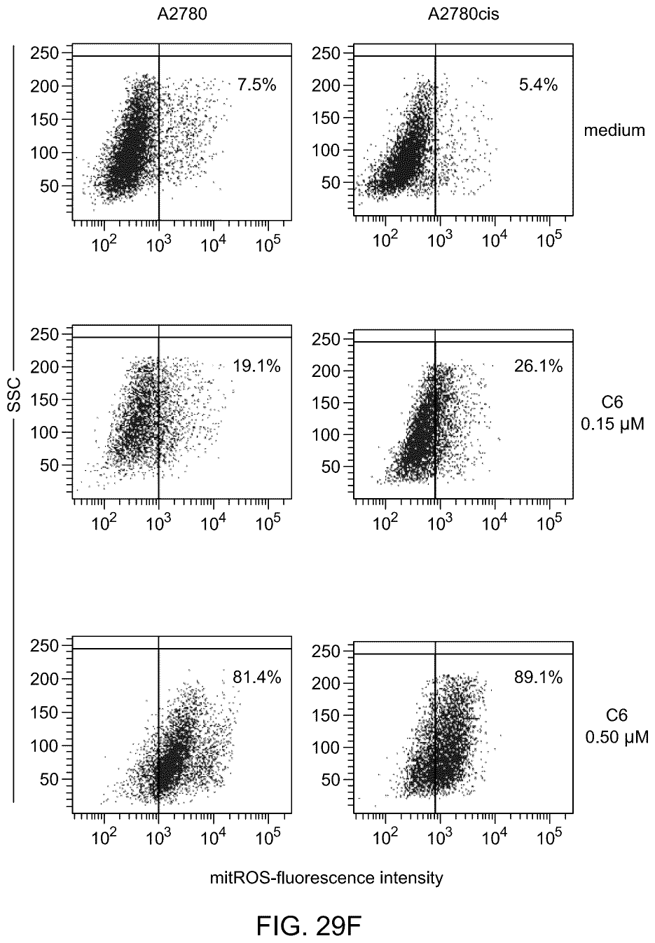

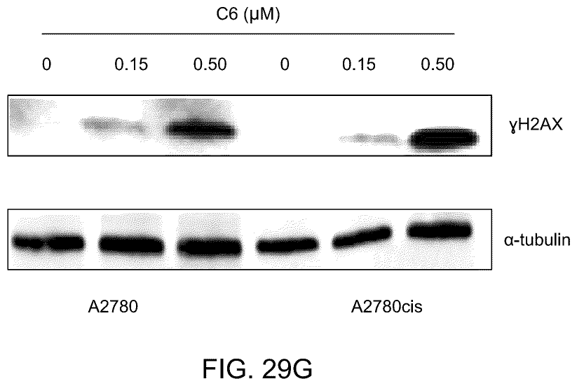

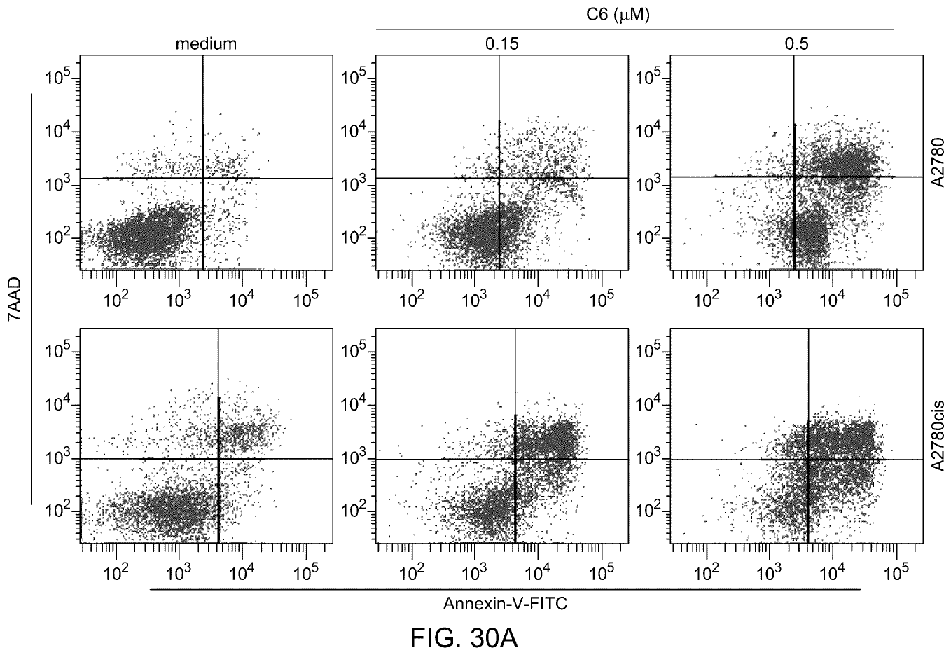

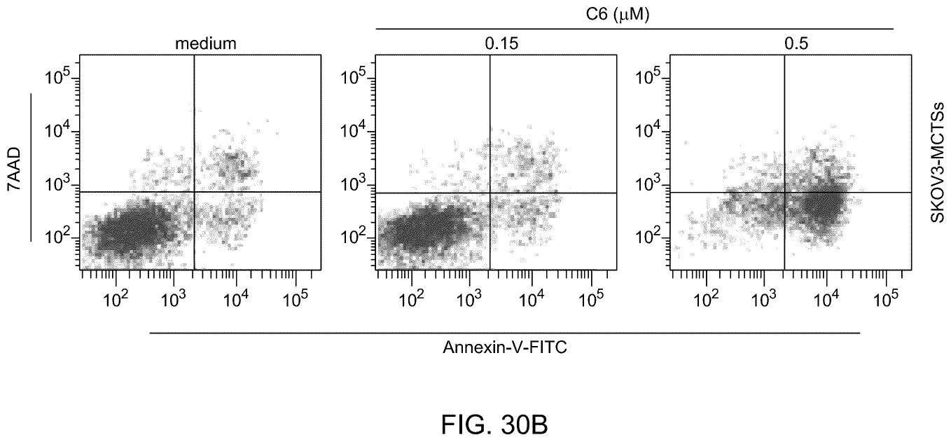

Treatment of A2780 and A2780cis with complex 6 (IC₅₀ and IC₇₅) increased in a dose-dependent manner the percentage of annexin-V-positive cells (early apoptosis) and of double stained annexin-V and 7AAD-positive cells (late apoptosis) (FIG. 29A and FIG. 30A). Consistently, complex 6 activated caspase-3,7 (FIG. 29B), induced the proapoptotic BAX (FIG. 29C), and down-regulated the antiapoptotic BCL-2 (FIG. 29D), indicating the involvement of apoptosis in the anticancer activity of complex 6.

High levels of reactive oxygen species (ROS) can cause cell death by inducing apoptosis or DNA damage. Complex 6 increased mitochondrial ROS (mitROS) generation in a dose-dependent manner (FIG. 29E). Representative dot plots showing the percent of mitROS-positive cells after complex 6 treatment are shown in FIG. 29F. DNA damage can be evaluated by the formation of DNA double-strand breaks (DSBs). DSBs rapidly result in the phosphorylation of histone H₂AX (γH2AX), a sensitive marker used to examine DNA damage. Complex 6 induced DNA-DSBs as demonstrated by the phosphorylation of histone H₂AX (γH2AX), as can be observed in FIG. 29G.

### Activity of Complex 6 in 3D-SKOV3-MCTSs: Proliferation and Mechanism of Action

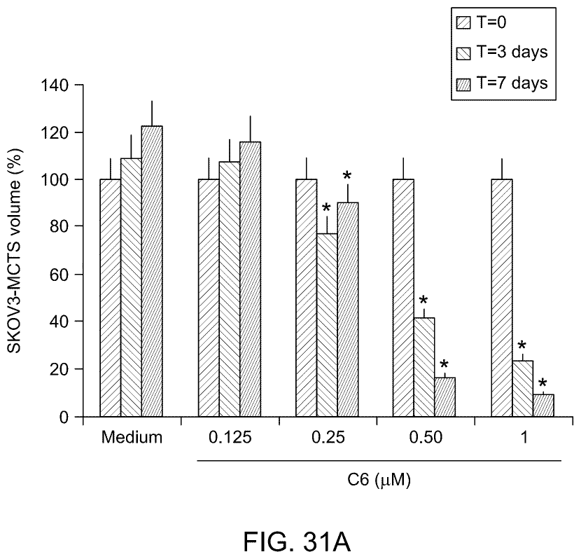

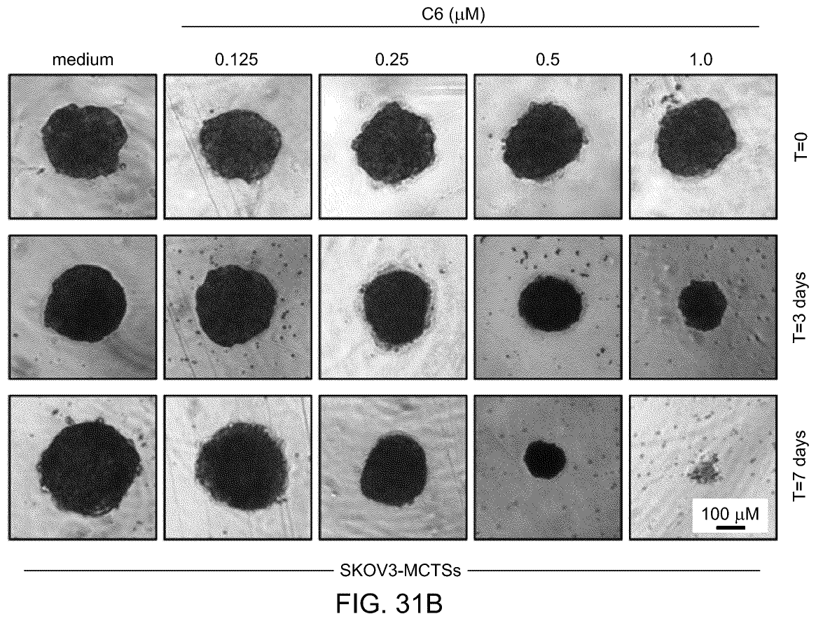

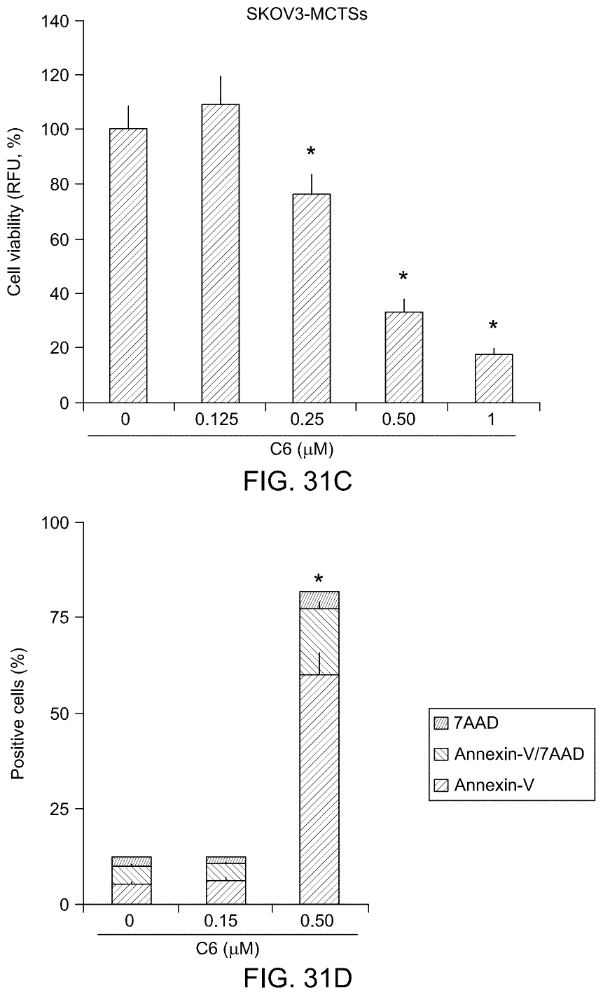

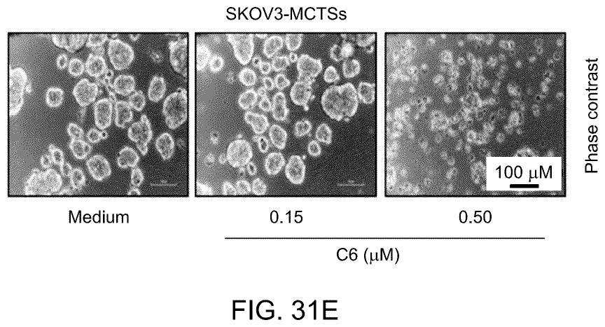

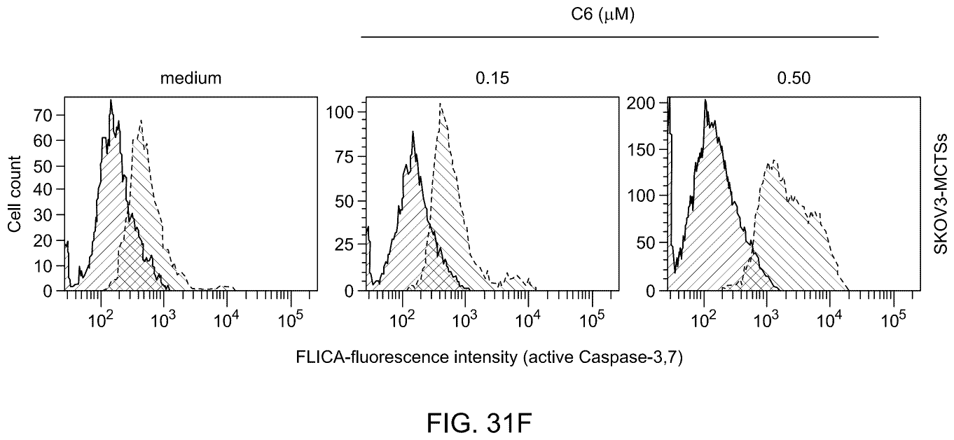

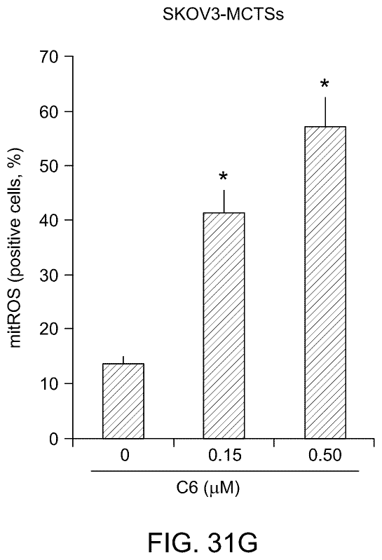

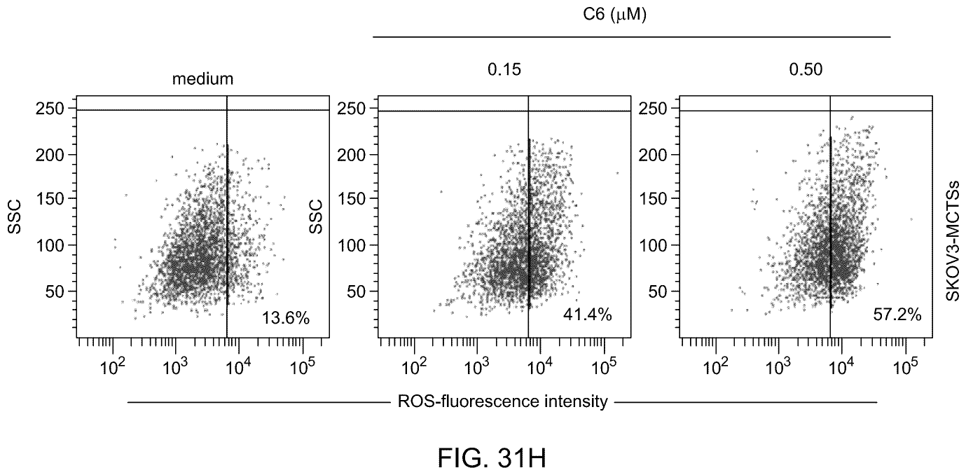

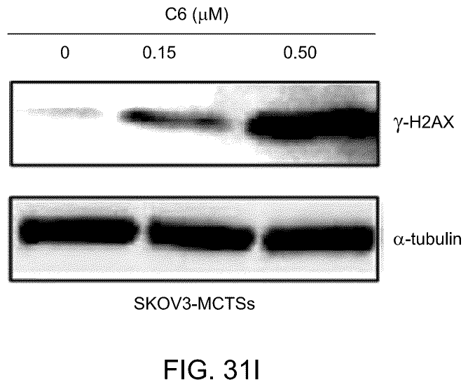

Cytotoxic effects on MCTSs are obtained with drugs exhibiting high accumulation, penetration, and stability. Compounds with phosphane ligands had an increased cellular uptake and were active in three-dimensional MCTSs, which were a reliable tumor model for in vitro drug testing, and were used to study drug penetration. The effects of complex 6 in MCTSs from SKOV3 cells (SKOV3-MCTSs) were evaluated by generating four-day-old single SKOV3-MCTSs with defined size and treating them with increasing concentrations of complex 6. Treatment for 3 days with complex 6 (0.5 μM) decreased SKOV3-MCTS volume by about 60% with respect to a control, and decreased SKOV3-MCTS volume by about 75% after 7 days of treatment (FIG. 31A and FIG. 31B). At the concentration of 1 μM, complex 6 almost entirely diminished the SKOV3-MCTSs, likely due to phosphane ligands making complex 6 more lipophilic, resulting in its increased cellular uptake by tumor spheroids. Representative phase contrast microphotographs demonstrate the reduction of SKOV3-MCTS volumes by complex 6, which are shown in FIG. 31B. PrestoBlue cell viability reagent was used to evaluate the viability of SKOV3-MCTSs with multiple sizes treated for 72 hours with complex 6 (FIG. 31C). Apoptosis (annexin-V-FITC/7AAD staining and caspase-3,7 activation), mitROS generation, and DNA-DSBs in SKOV3-MCTSs were evaluated. Spheroids were collected and disaggregated into a single cell suspension to perform both cytofluorimetric and western blot assays after treatment of SKOV3-MCTSs with complex 6. It was observed that complex 6 caused an increase of annexin-V+ cells (FIG. 31D, FIG. 30B). Representative phase-contrast microphotographs showing the cytotoxic effects of complex 6 in multiple size SKOV3-MCTSs are shown in FIG. 31E. Complex 6 caused the activation of caspase-3,7 (FIG. 31F), increased mitROS generation (FIG. 31G and FIG. 31H), and induced DNA-DSBs, as evaluated by TH2AX western blot assay (FIG. 31I). ROS increase support the observed DNA damage and caspase activation by complex 6.

### Effects of Complex 6 on Cell Cycle Phases and Cell- Cycle-Related Proteins

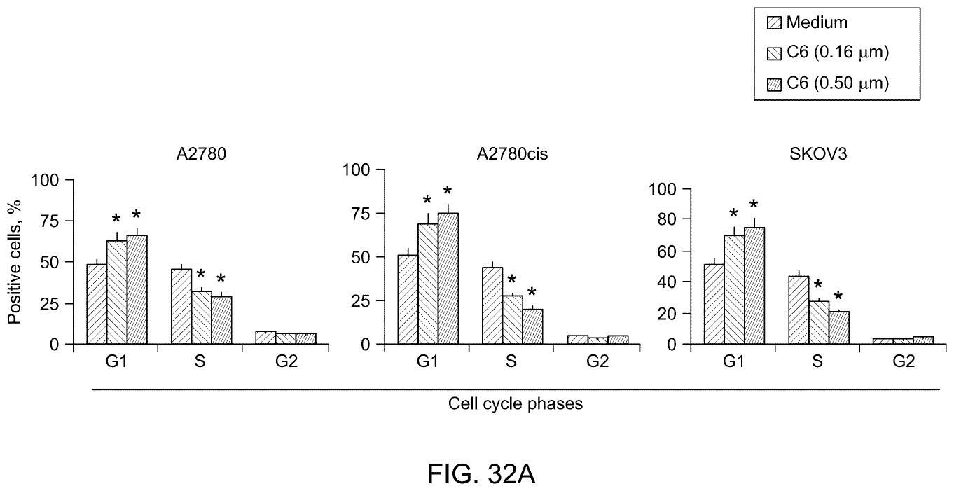

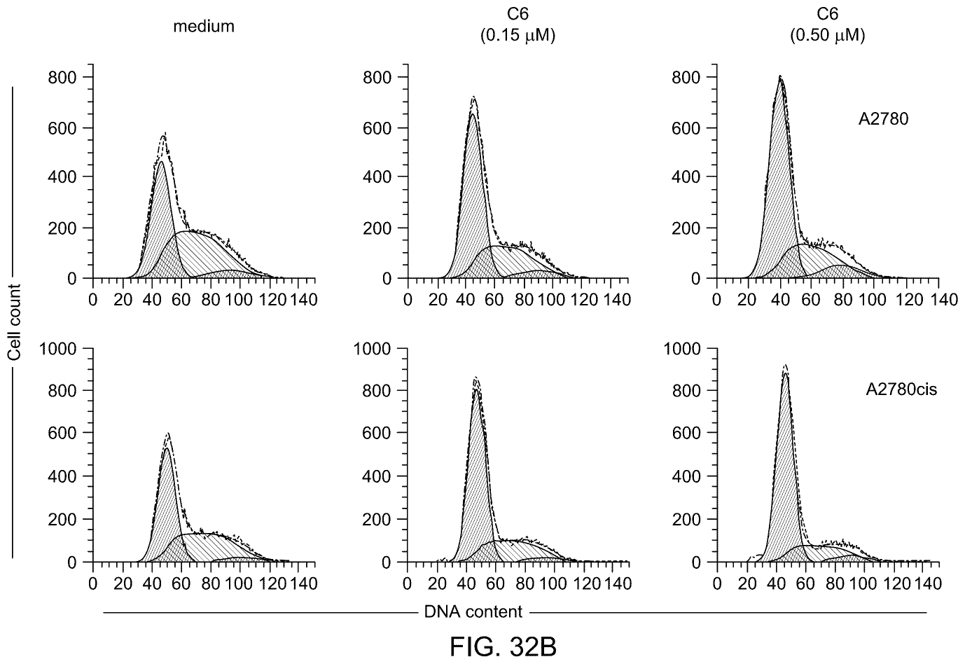

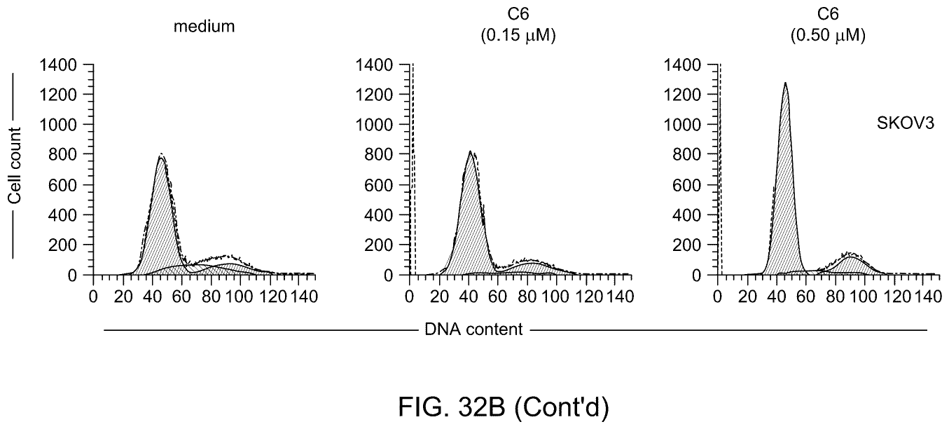

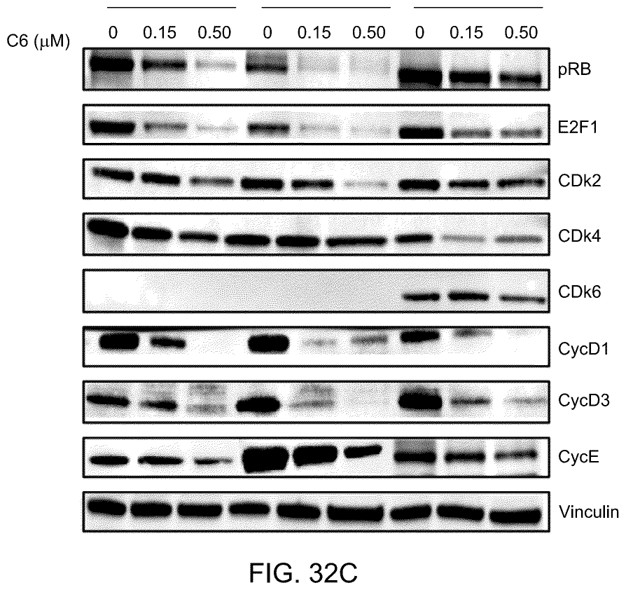

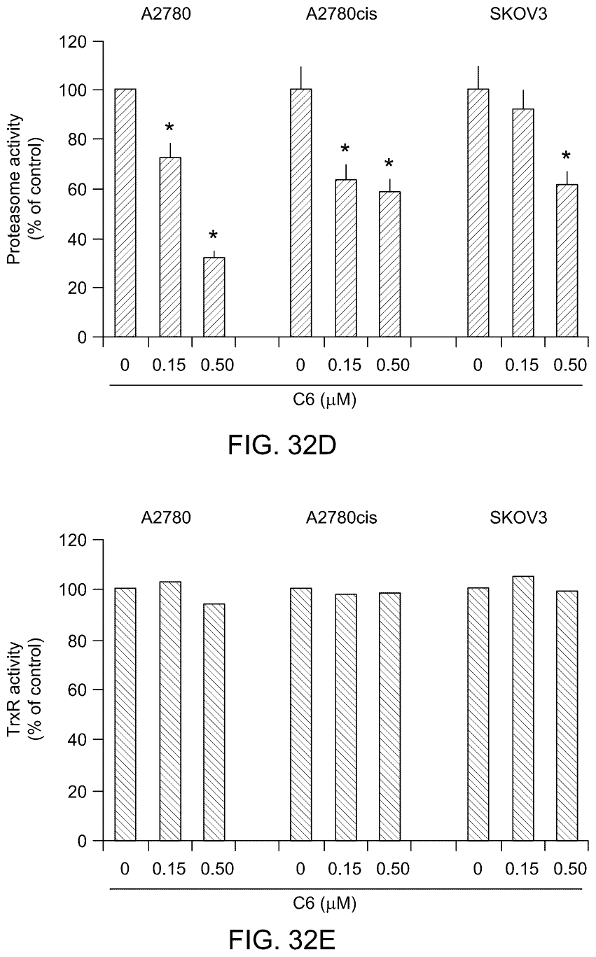

Cell cycle checkpoints function as DNA surveillance mechanisms to avoid genetic errors during cell division. They can stop cell cycle progression and induce cell cycle exit or cell death in response to irreparable DNA damage. To investigate the mechanism underlying complex 6-mediated activity, A2780, A2780cis, and SKOV3 cells were treated for 24 hours with complex 6 (0.15 μM and 0.5 μM), and then cell cycle phases were analyzed by flow cytometry. It was observed that complex 6 induced an accumulation in the G0/G1 phase of the cell cycle and a decrease in the S phases (FIG. 32A). The representative flow cytometry histograms of cell cycle phase modifications after treatment with complex 6 are shown in FIG. 32B. The cell cycle regulatory proteins implicated in the G1/S phase progression were assessed for further cell cycle analysis.

The following proteins were analyzed by a Western blot: CDk2, CDk4, CDk6, CycE, CycD1, CycD3, which controls the G1/S phase progression; the transcription factor, E2F1, and the tumor suppressor protein, retinoblastoma (pRB), key regulators of the progression through checkpoints at G1/S, Foxo3A, pGSK-3β; negative regulators of CycD, Cdc25a, PI3KI, pERK1/2, and bRAF; and positive regulators of cyclin D.

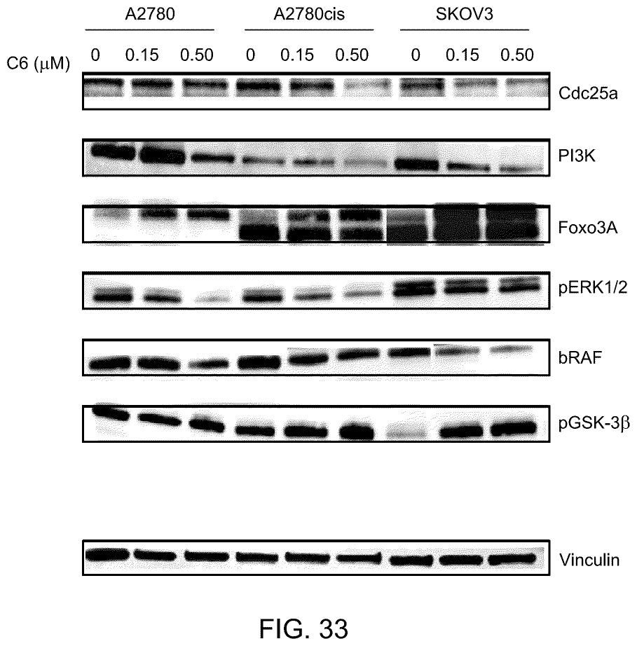

Consistent with the accumulation of OvCa cells in G0/G1 phase and the decrease of the S phase, treatment with complex 6 down-regulated the expression of CDk2, CDk4, CDk6, CycE, CycD1, CycD3 (FIG. 32C) and of the activators E2F1 and pRB. Complex 6 increased the expression of Foxo3A and pGSK-30 and decreased that of Cdc25a, PI3KI, pERK1/2, and bRAF (FIG. 33).

These data indicate that complex 6 promotes the accumulation of OvCa cells in the G0/G1 phase (cell cycle arrest) by modulating the expression of proteins involved in cell cycle regulation, thus contributing to its anticancer activity.

### Effects of Complex 6 on the Activity of the 20S Proteasome and TrxR

A target of various gold complexes is a proteasome, which is a central factor of the protein degradation apparatus, namely, the ubiquitin-proteasome system (UPS). The UPS is involved in the turnover of most cellular soluble proteins, playing fundamental roles in regulating many cellular activities, such as cell cycle, apoptosis, DNA repair, cell adhesion, and the like. It was observed that complex 6 decreased the 20S proteasome activity in a dose-dependent manner in OvCa cells, especially in A2780 cells (FIG. 32D). TrxR, which regulates the cellular redox balance, is also a target of gold complexes. Complex 6 did not modify its enzymatic activity in OvCa cells (FIG. 32E). The increased ROS generation could result from proteasome inhibition.

### Effects of Cisplatin on OvCa Cells

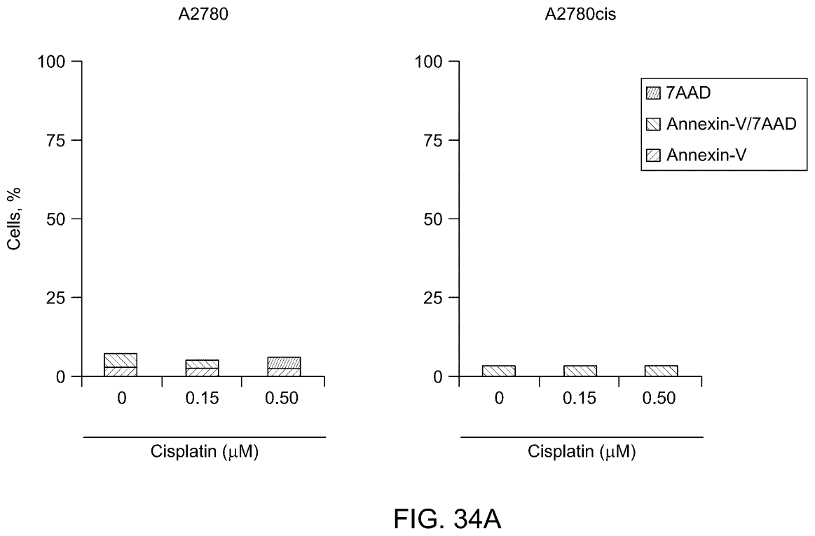

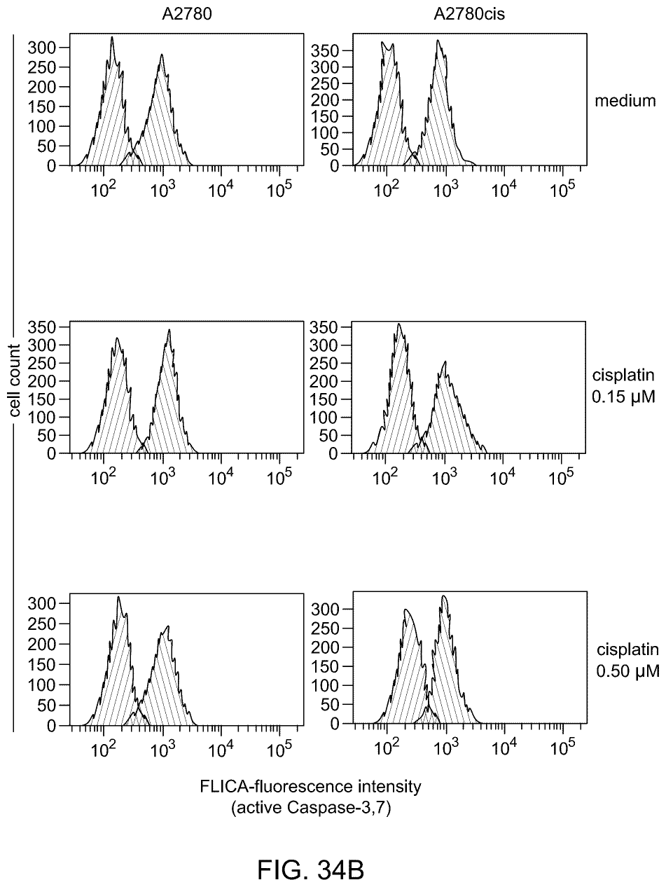

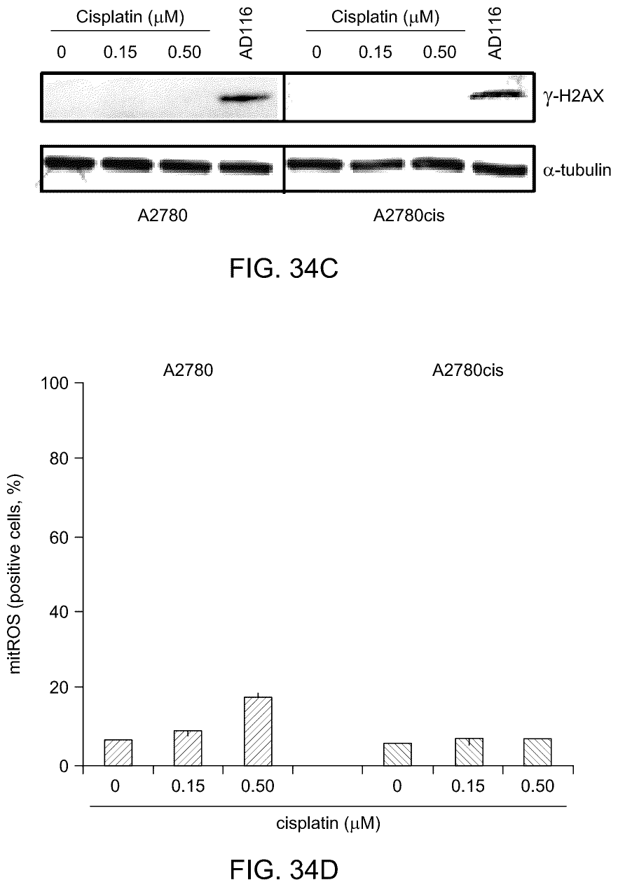

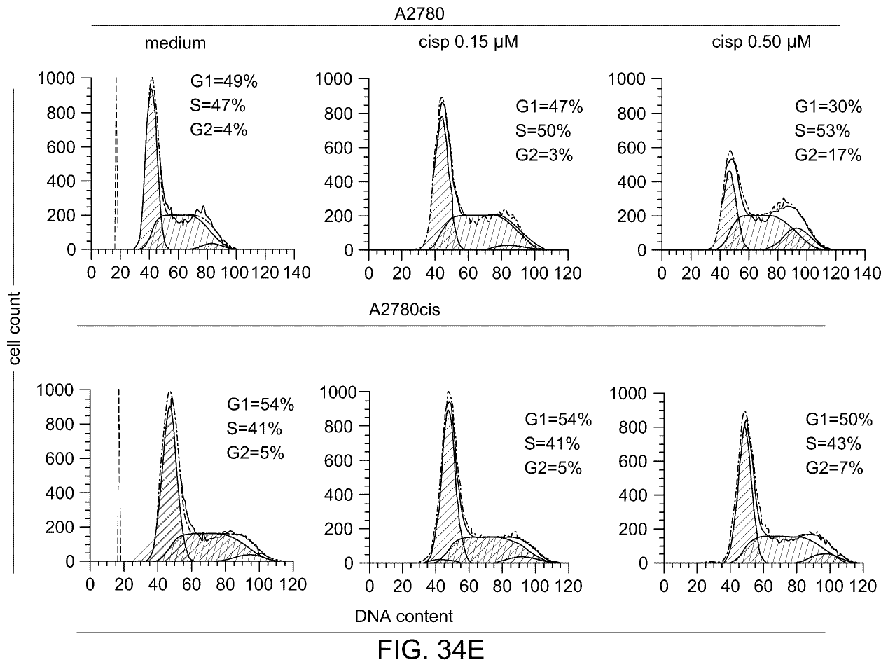

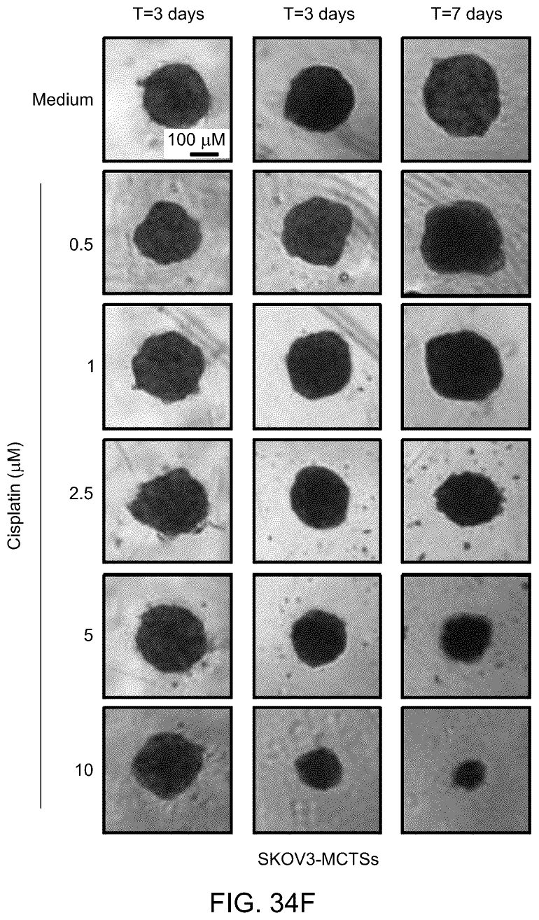

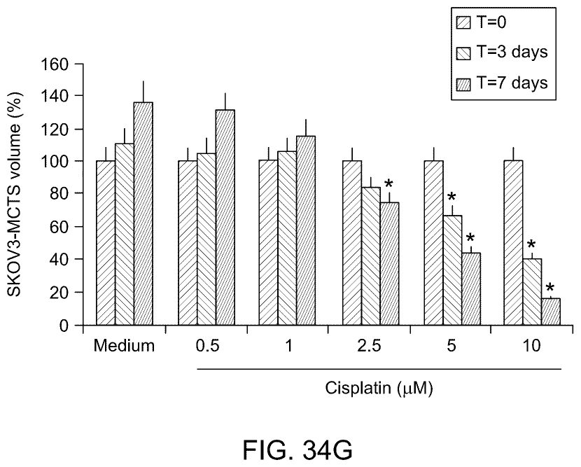

To further investigate the advantage of complex 6 to cisplatin (Table 8), the cisplatin effects on apoptosis (annexin-V/7AAD staining and caspase-3,7 activation), ROS generation, DNA-DSBs (YH2AX), cell cycle phases, and spheroids growth were studied. Cisplatin, used at the same concentrations of complex 6 (0.15 μM and 0.50 μM), did not induce apoptosis (FIG. 34A and FIG. 34B) and DNA-DSBs (FIG. 34C). The highest concentration of cisplatin (0.50 μM) induced ROS generation (from 6.4% to 17.4%) (FIG. 34D) and caused a slight increase of the G2/M phase of the cell cycle (FIG. 34E) in the cisplatin sensitive A2780 cells, A2780cis. SKOV3-MTCS growth after treatment with cisplatin (0.5-10 μM) was evaluated. Cisplatin (1 μM, 7 days) caused a slight decrease of SKOV3-MCTS growth (16% with respect to control) (FIG. 34F and FIG. 34G),compared to the same concentration of complex 6, which completely destroyed spheroids (FIG. 31A and FIG. 31B). Cisplatin used at its SKOV3-IC₅₀ concentration (˜5 μM, 3 days) demonstrated an MCTS growth reduction (FIG. 34F and FIG. 34G); however, complex 6 was more potent at its SKOV3-IC₅₀ concentration (0.5 μM) (FIG. 31A and FIG. 31B). These data support that complex 6 is more active than the reference drug cisplatin in 2D OvCa cell cultures and 3D SKOV3-MCTSs.

### Effects of Auranofin on OvCa Cells

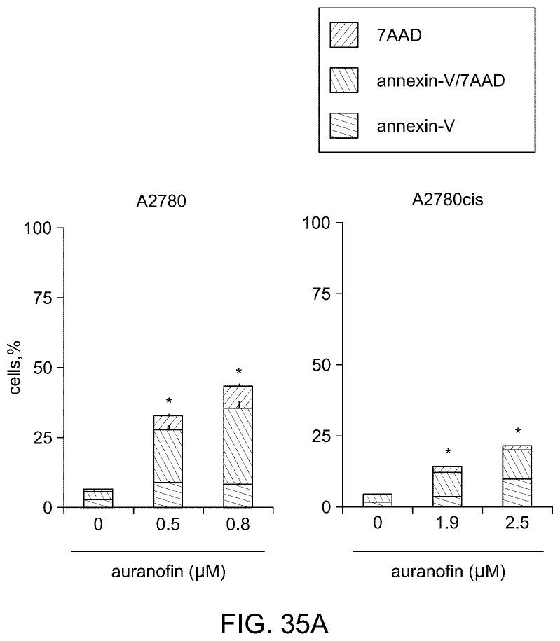

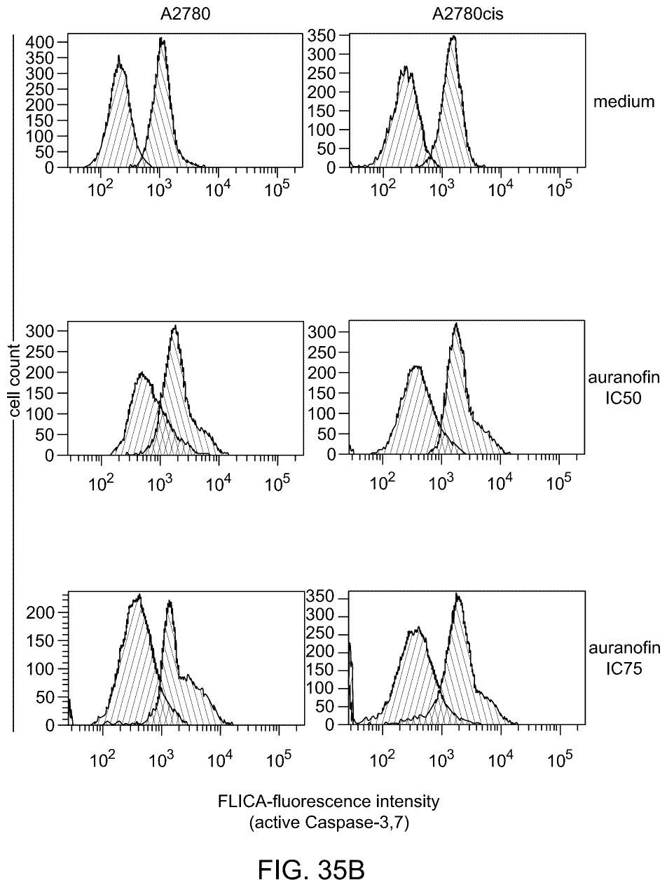

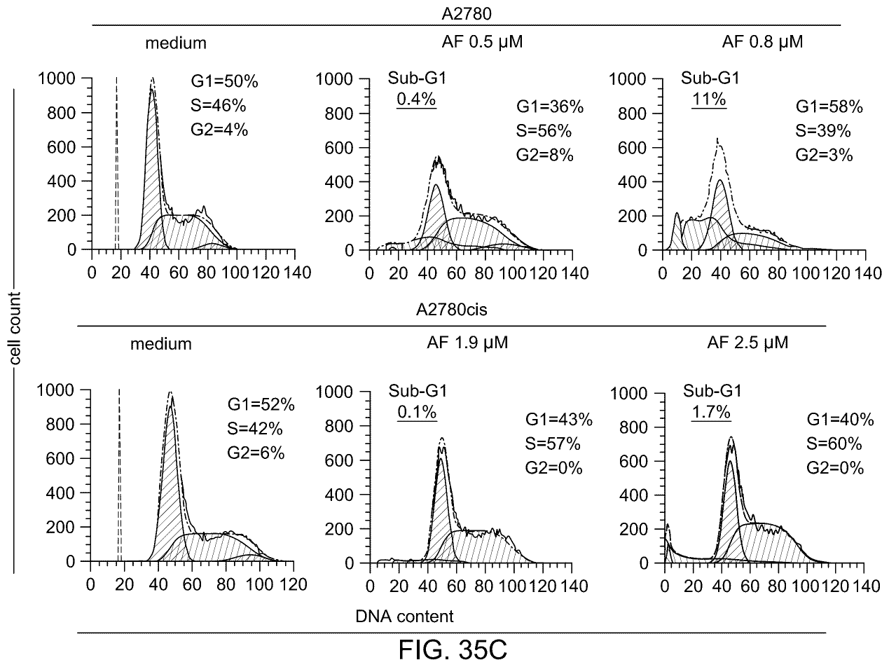

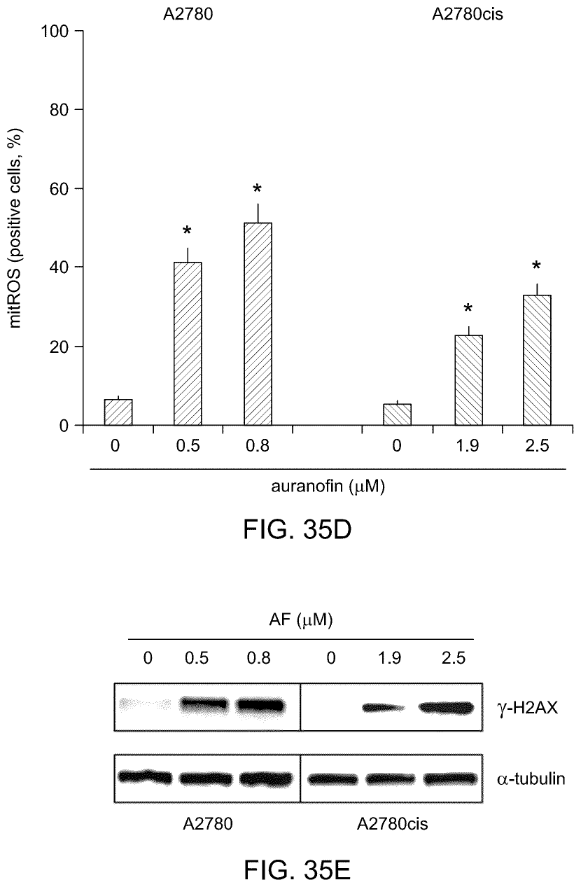

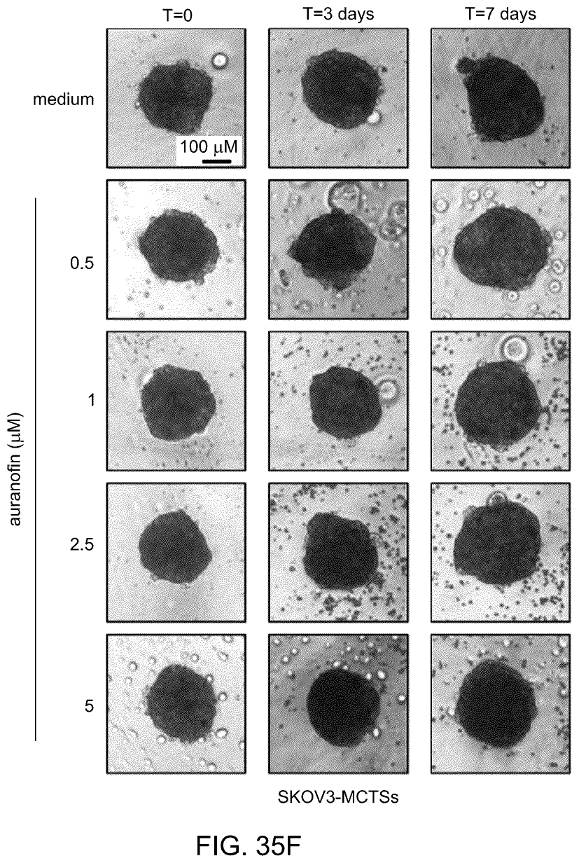

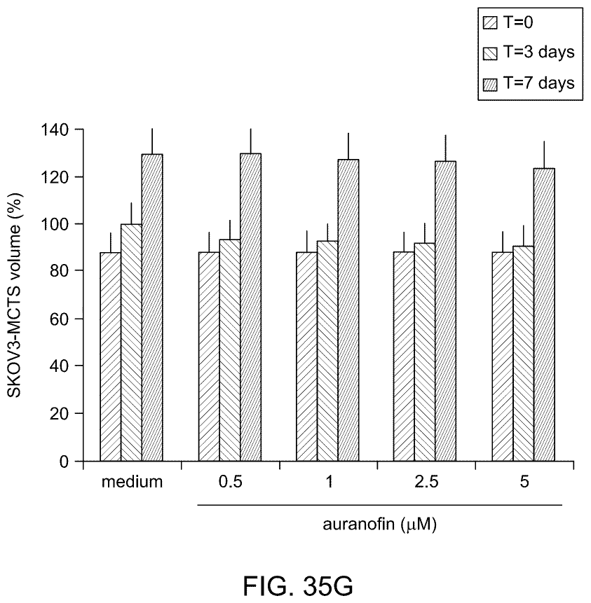

For comparative purposes, the mode of action of the triethyl phosphine-gold(I) auranofin in A2780 and A2780cis used at its IC₅₀ and IC₇₅ concentrations (Table 8, FIG. 27C) were studied. Auranofin was less active in inducing apoptosis in cisplatin-resistant A2780cis than in A2780 cells, as evaluated by annexin-V/7AAD staining (FIG. 35A) and caspase-3,7 activation (FIG. 35B). In A2780 cells, auranofin (0.5 μM, IC₅₀) decreased the G1 phase (FIG. 35C) and at the highest concentration (0.8 μM, IC₇₅) induced DNA fragmentation (sub-G₀/G₁ phase=11%). In A2780cis, auranofin slightly decreased the G1 phase and increased the S phase (FIG. 35C). At the highest concentration (2.5 μM, IC₇₅), it caused low DNA fragmentation (sub-G0/G1 phase=1.7%) (FIG. 35C). Auranofin induced ROS generation (FIG. 35D) and DNA strand breaks (FIG. 35E) and was more active in A2780 than in A2780cis cells.

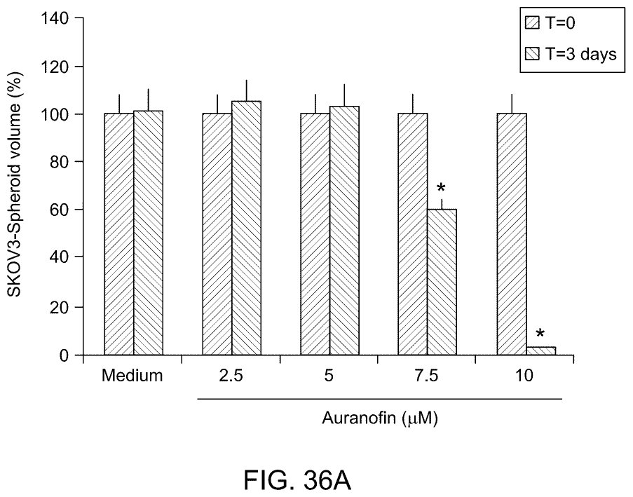

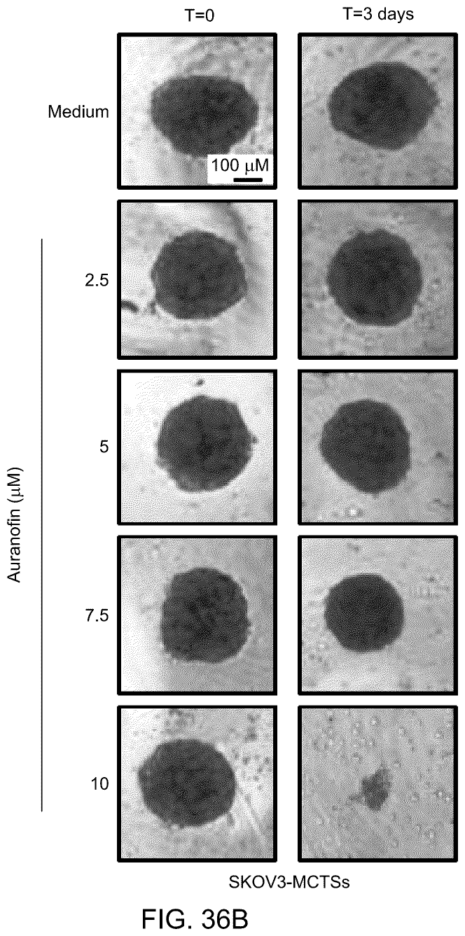

The SKOV3-MTCS growth (volume) was evaluated after treatment with auranofin (0.5-5 μM) (Table 8). While complex 6 (1 μM) completely destroyed SKOV3-MCTSs (FIG. 31A and FIG. 31), the same concentration of auranofin did not reduce SKOV3-MCTS volume (FIG. 35G and FIG. 35F). In addition, complex 6 was more active than auranofin when used at its SKOV3-IC₅₀ concentration (0.5 μM vs 2.5 μM) (FIG. 31A and FIG. 31B; and FIG. 35G and FIG. 35F). It was observed that SKOV3-MCTS growth inhibition by auranofin was seen at concentrations higher than 5 μM (FIG. 36A and FIG. 36B).

These results support that auranofin (FR=3.7) is less active than complex 6 (FR=0.86) in counteracting cisplatin resistance, inducing apoptosis and ROS generation, and reducing SKOV3-MCTSs volume. Auranofin slightly affected the cell cycle. It reduced the G1 phase and caused DNA fragmentation, whereas complex 6 caused the accumulation of OvCa cells in the G1 phase.

### Effects of Complex 6 on Cell Migration and Cytoskeleton Organization

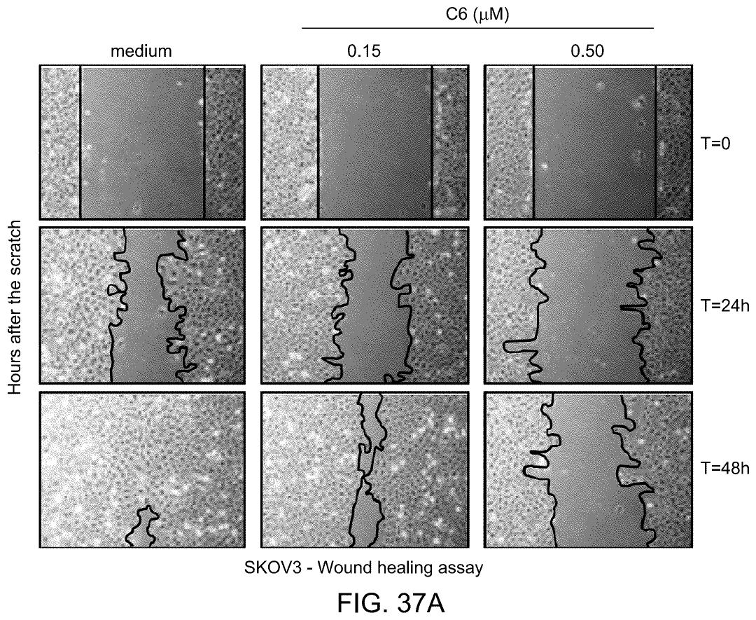

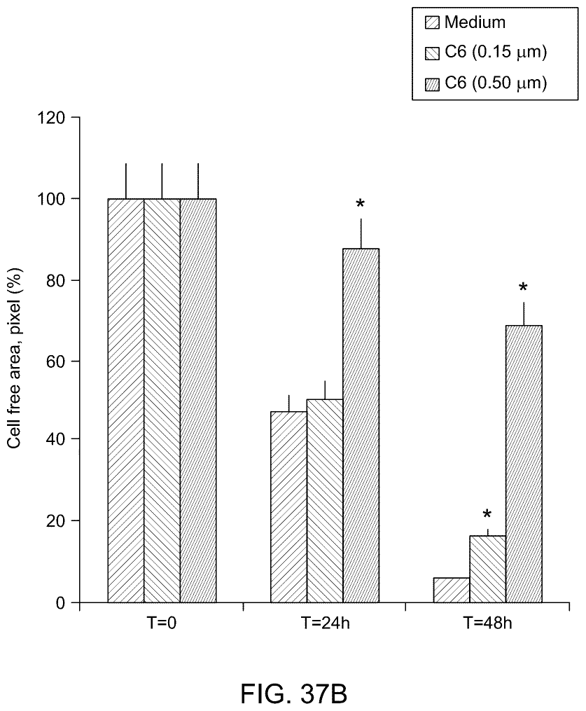

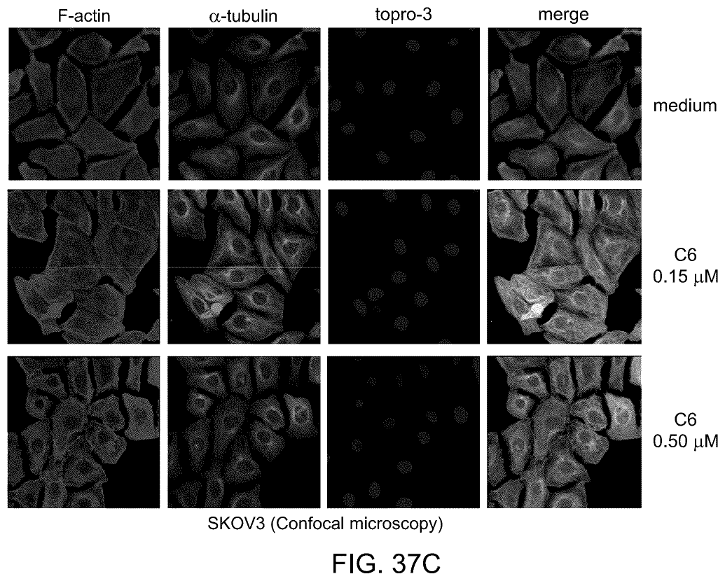

High-grade serous OvCa accounts for the majority of OvCa cases and has the lowest survival rates and high invasive potential. Thus, a successful therapy should prevent not only the tumor growth but also the metastatic potential of cancer cells. To evaluate the migration of SKOV3 cells, a wound healing assay was performed. The SKOV3 cells were treated for 24 hours with complex 6, washed, scraped to create a “wound,” and then cultured with a drug-free medium (FIG. 37 Å). Treatment with complex 6 decreased the SKOV3 migration. The capability of cells treated with complex 6 to refill the empty area (“wound”) of the monolayer was greater compared to the capability of the untreated cells (FIGS. 37A and 37B). Forty-eight hours after the monolayer was scratched, the remaining uncovered area was about 5% in control cells and 70% at the highest concentration of complex 6 (FIGS. 37A and 37B). Recently, N-heterocyclic carbene-gold(I) complexes were found to regulate actin polymerization. Thus, it was further investigated if the observed decreased migration by complex 6 could be associated with modifications of cytoskeleton organization. Microtubules and actin filaments are cytoskeletal components that have roles in cell signaling and division and regulate cell movement. Thus, the spatial organization of both F-actin and α-tubulin by immunofluorescence confocal microscopy in SKOV3 cells treated for 24 hours with complex 6 (FIG. 37C) was evaluated. It was observed that in untreated SKOV3 cells, the F-actin filaments exhibited a normal distribution and architecture. Conversely, complex 6-treated SKOV3 cells exhibited an irregular disposition and organization of the F-actin cytoskeleton (FIG. 37C). The F-actin system appeared more compact, not equally distributed, and dot-like structures became visible (FIG. 37C). Complex 6 did not modify α-tubulin distribution (FIG. 37C). These results indicate that complex 6 may affect not only OvCa growth but also, it's metastatic spreading.

### Effects of Complex 6 in SKOV3-Xenografts Growth

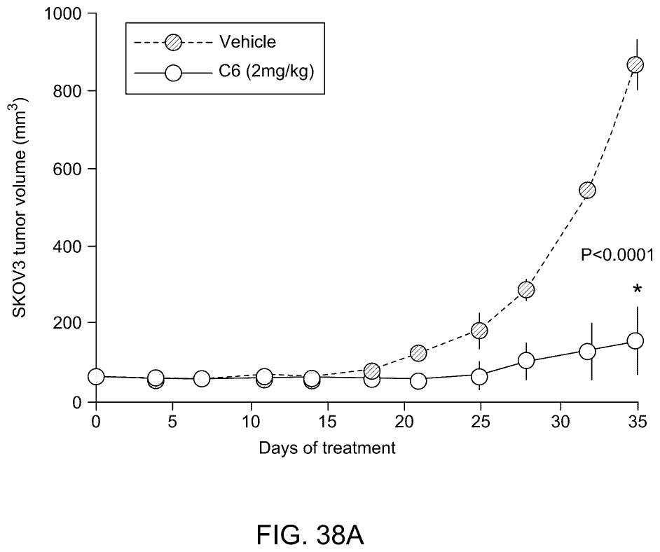

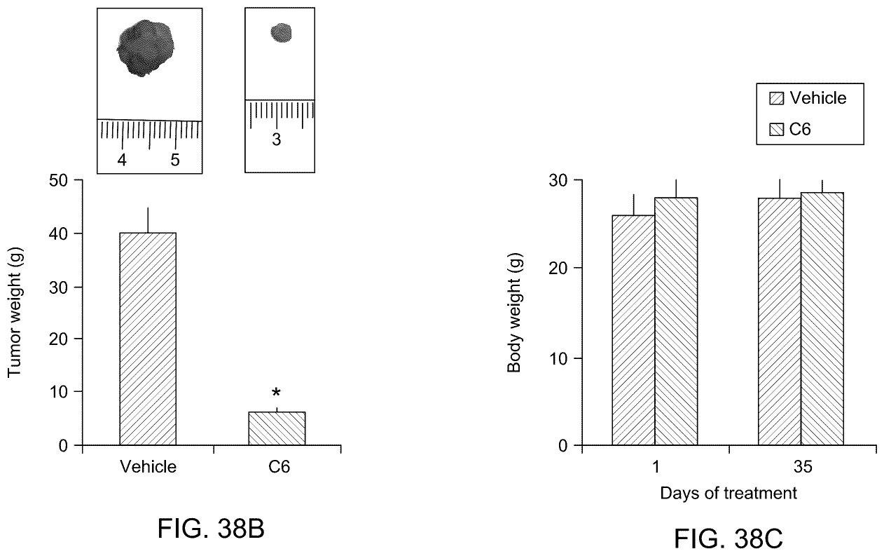

In vivo experiments were performed and the antitumor activity of complex 6 in tumor xenografts derived from SKOV3 cells (10×10⁶ cell/mouse) injected subcutaneously in 10 female athymic nude NU(NCr)-Foxn1^{nm} mice was evaluated. Animals were treated with two intratumoral injections/week of complex 6 (2 mg/kg) or vehicle (10% DMSO, 20% Cremophor, 70% PBS). Complex 6 caused a reduction of more than 80% of tumor volume with respect to vehicle-treated mice (FIG. 38A). At day 35, control tumors had grown to a mean volume of 869.5±61.9 mm³, whereas complex 6-treated tumors to 155.8±84.5 mm³ (FIG. 38A). Consistent with the decrease in tumor volume, tumor mass weight was reduced in mice treated with complex 6 (FIG. 38B). Representative images of extracted tumors are shown in FIG. 38B. Complex 6 treated animals were normal to physical inspection with a comparable weight with respect to untreated mice (FIG. 38C).

To conclude, the synthesis and characterization of seven new gold(I) complexes (1-7) based on phosphanes and an N-heterocyclic carbene and their anticancer properties against OvCa cell lines models were described. The X-ray structure of complex 6 revealed an almost perfectly linear coordination at the gold(I) center. The gold(I) complexes exerted a potent cytotoxicity with comparable antitumor effects against OvCa cells sensitive and resistant to cisplatin, indicating that they may counteract cisplatin resistance. Among the series, complex 6 was found to show the highest activity, and therefore, the mechanism of action and the in vivo activity of this complex were evaluated in A2780, A2780cis, and SKOV3 OvCa cells, which are capable of forming 3D multicellular tumor spheroids and are resistant to several antitumoral drugs, including cisplatin. Complex 6 decreased OvCa cell viability in 2D monolayer cultures and in the 3D SKOV3-MCTSs and was more toxic in tumor cells than in healthy human stromal cells. Complex 6 induced apoptosis, activated caspase-3,7, increased mitROS generation, and caused DNA damage (DSBs) in A2780, A2780cis, and 3D-SKOV3-MCTSs, possibly for the presence of the phosphane ligand. Consistent with the decreased expression of cyclins and CDKs involved in the G1/S phase progression of the cell cycle, complex 6 blocked cells in the G0/G1 phase. Complex 6 was more active in 2D and 3D models of OvCa than the reference drug cisplatin and the gold(I) phosphine auranofin. Complex 6 did not alter the TrxR enzymatic activity of OvCa cells, but it decreased the 20S proteasome activity. It slowed cell migration and modified actin polymerization and distribution. Complex 6 showed antitumor activity in SKOV3 tumor xenograft. The results obtained in the present disclosure may serve as the starting point for deeper preclinical investigations of these new gold(I) complexes, also in other cancer models.

Numerous modifications and variations of the present disclosure are possible in light of the above teachings. It is therefore to be understood that within the scope of the appended claims, the invention may be practiced otherwise than as specifically described herein.

1: A complex, comprising:
gold;
a 1,3-bis(2,6-di-isopropylphenyl)imidazole-2-ylidene ligand;
and at least one phosphane ligand selected from the group consisting of bis(2-cyanoethyl)phenylphosphane, (1R,2R)-2-diphenylphosphano)-1-aminocyclohexane, (1R,2R)-2-diphenylphosphano)-1,2-diphenylethylamine, (R)-2-(diphenylphosphano)-1-phenylethylamine, (R)-1-(diphenylphosphano)-2-amino-3,3-dimethylbutane, tricyclohexylphosphane, and 3-(diphenylphosphano)propylamine),
wherein the 1,3-bis(2,6-di-isopropylphenyl)imidazole-2-ylidene ligand and the at least one phosphane ligand are bonded to the gold.

2: The complex of claim 1, wherein the phosphane ligand is tricyclohexylphosphane.

3: The complex of claim 1, having a half maximal inhibitory concentration of 0.1 to 2.5 μM in a first ovarian cancer cell line, A2780.

4: The complex of claim 1, having a half maximal inhibitory concentration of 0.1 to 4.0 μM in a first ovarian cancer cell line resistant to cisplatin, A2780cis.

5: The complex of claim 1, having a fold resistance of 0.5 to 2, wherein the fold resistance is based on a ratio of a half maximal inhibitory concentration in the first ovarian cancer cell line resistant to cisplatin, A2780cis, to a half maximal inhibitory concentration in the first ovarian cancer cell line, A2780.

6: The complex of claim 2, having a half maximal inhibitory concentration of 0.10 to 0.20 μM in the first ovarian cancer cell line, A2780.

7: The complex of claim 2, having a half maximal inhibitory concentration of 0.10 to 0.15 μM in the first ovarian cancer cell line resistant to cisplatin, A2780cis.

8: The complex of claim 2, having a fold resistance of less than 1, wherein the fold resistance is based on a ratio of a half maximal inhibitory concentration in the first ovarian cancer cell line resistant to cisplatin, A2780cis, to a half maximal inhibitory concentration in the first ovarian cancer cell line, A2780.

9: The complex of claim 2, having a half maximal inhibitory concentration of 0.55 to 0.65 μM in a second ovarian cancer cell line, SKOV3.

10: A pharmaceutical composition comprising the complex of claim 1 in an amount effective for treating a patient having ovarian cancer, and at least one pharmaceutical additive or adjuvant.

11: The complex of claim 1, made by a process comprising:
mixing a silver hexafluorophosphate salt in a polar protic solvent with a 1,3-bis(2,6-diisopropylphenyl-imidazole-2-ylidene) gold chloride salt in a polar aprotic solvent to form a reaction mixture;
reacting the reaction mixture with the phosphane ligand in a polar aprotic solvent to form the complex,
wherein the silver hexafluorophosphate salt, the 1,3-bis(2,6-diisopropylphenyl-imidazole-2-ylidene) gold chloride salt, and the phosphane ligands are in an equimolar amount.

12: A method for treating ovarian cancer, comprising:
administering the complex of claim 2 to a patient in need of treatment for ovarian cancer,
wherein during the administering the complex is contacted with an ovarian cancer in the form of at least one of a monolayer of cells, a multicellular tumor spheroid (MCTS), and an in vivo xenograft tumor.

13: The method of claim 12, wherein administering the complex induces apoptosis in A2780, A2780cis, and a multicellular tumor spheroid sample of the second ovarian cancer cell line, SKOV3.

14: The method of claim 12, wherein the complex administered at a concentration of 1 μM decreases the volume of SKOV3-MCTSs by 85 to 95% based on volume after 7 days.

15: The method of claim 12, wherein the complex administered at a concentration of 0.50 μM generates mitochondrial reactive oxygen species (mitROS) in SKOV3-MCTSs of 50 to 60% based on a cell count after 24 hours.

16: The method of claim 12, wherein the complex administered to the sample at a concentration of 0.1 to 0.6 μM has an amount of cells in the S phase from 15 to 35% compared to the sample in the absence of the complex having an amount of cells in the S phase from 40 to 50% based on a cell count after a time of 24 hours.

17: The method of claim 12, wherein the complex administered to the sample at a concentration of 0.1 to 0.6 μM has an amount of cells in the G1 phase from 60 to 80% compared to the sample in the absence of the complex having an amount of cells in the G1 phase from 45 to 55% based on a cell count after a time of 24 hours.

18: The method of claim 12, wherein the complex administered to the sample at a concentration of 0.5 μM decreases a proteasome activity by 30 to 70% after 24 hours based on a 20S-proteasome assay compared to the sample in the absence of the complex.

19: The method of claim 12, wherein the sample is in vivo xenograft tumor and the complex administered to the sample showed a reduction of the sample of more than 80% based on sample volume after 35 days of a treatment of 2 milligrams of the complex per kilogram of a body weight of a host of the sample two times a week.

20: A pharmaceutical composition comprising the complex of claim 1 or a pharmaceutically acceptable salt thereof, in combination with a pharmaceutically acceptable carrier, diluent, or excipient.
